SEQUENCE LISTING (6 sequences)

SEQ ID NO: 1 - DNA, 23 bp, Mus musculus
aagtgactgtgaaaacagaagca

SEQ ID NO: 2 - DNA, 22 bp, Mus musculus
gcagccatttgtaagggttgac

SEQ ID NO: 3 - DNA, 21 bp, Mus musculus
atccgctacatcgaaggtctg

SEQ ID NO: 4 - DNA, 21 bp, Mus musculus
ctcgacacagccgcactcttc

SEQ ID NO: 5 - DNA, 21 bp, Mus musculus
aggtcggtgtgaacggatttg

SEQ ID NO: 6 - DNA, 23 bp, Mus musculus
tgtagaccatgtagttgaggtca

(19) United States
(12) Patent Application Publication
(10) Pub. No.: US 2024/0001004 A1
(43) Pub. Date: Jan. 4, 2024

(54) MULTICOMPARTMENT CONDUCTIVE COLLAGEN SCAFFOLD AND RELATED METHODS OF MAKING AND USING THE SAME

(71) Applicant: University of Virginia Patent Foundation, Charlottesville, VA (US)

(72) Inventors: Steven R. Caliari, Charlottesville, VA (US); Ivan Basurto, Charlottesville, VA (US)

(73) Assignee: University of Virginia Patent Foundation, Charlottesville, VA (US)

(21) Appl. No.: 18/037,684

(22) PCT Filed: Nov. 18, 2021
(86) PCT No.: PCT/US2021/059949

§ 371 Date: May 18, 2023

Related U.S. Application Data
(60) Provisional application No. 63/115,199, filed on Nov. 18, 2020.

(51) Int. Cl. A61L 27/56; A61L 27/38; A61L 27/26; A61N 1/05

(52) U.S. Cl. CPC A61L 27/56; A61L 27/3826; A61L 27/3834; A61L 27/26; A61N 1/05; A61L 2430/30

(57) ABSTRACT

A multicompartment conductive collagen scaffold composite, comprising a scaffold comprising collagen and an electrically conductive material, optionally wherein the electrically conductive material comprises electrically conductive particles, and further comprising longitudinally aligned pores, and methods of making and using the same.

### RELATED APPLICATIONS

The presently disclosed subject matter claims the benefit of U.S. Provisional Patent Application Ser. No. 63/115,199, filed Nov. 18, 2020, the disclosure of which is incorporated herein by reference in its entirety.

### STATEMENT OF GOVERNMENT INTEREST

This invention was made with government support under Grant No. AR075181 awarded by The National Institutes of Health. The government has certain rights to this invention.

### TECHNICAL FIELD

The presently disclosed subject matter relates in some embodiments to methods and compositions for tissue engineering. In particular embodiments, the presently disclosed subject matter provides multicompartment conductive collagen scaffold composite compositions, and methods of making and using the same.

### BACKGROUND

Volumetric muscle loss (VML) injuries result in permanent loss of muscle function that is debilitating to both civilian and military patient populations (Grogan & Hsu, 2011; Corona et al., 2015). Current approaches to treating severe VML injuries have had limited success, generally requiring multiple surgical interventions and often resulting in poor levels of functional improvement (Corona et al., 2013). While tissue engineering approaches have shown some promise for treatment of simple muscle defects, clinically relevant VML injuries are often compounded by damage to multiple tissues including connective and nervous tissue (Owens et al., 2008; Gilbert-Honick & Grayson, 2019). Particularly, damage to the peripheral nervous system can result in denervation and muscle atrophy, limiting the ability to generate force and execute normal movements (Carlson, 2014). The disruption of muscle fibers at the musculotendinous junction (MTJ), where most muscle injuries occur (Tidball et al., 1993), can further ablate the transfer of muscle-generated force to the skeletal system (Yang & Temenoff, 2009). Unfortunately, many VML repair strategies solely focus on skeletal muscle, neglecting surrounding tissues that are essential for function (Yang & Temenoff, 2009; Gilbert-Honick & Grayson, 2019). Despite this clear clinical need, therapeutic approaches to treat combined VML/MTJ injuries are lacking.

### SUMMARY

The presently disclosed subject matter provides in some embodiments a multicompartment conductive collagen scaffold composite. In some embodiments, the multicompartment conductive collagen scaffold composite comprises a scaffold comprising collagen and an electrically conductive material, optionally wherein the electrically conductive material comprises electrically conductive particles, and further comprising longitudinally aligned pores.

In some embodiments, the multicompartment conductive collagen scaffold composite further comprises a first compartment comprising a first collagen scaffold and an electrically conductive material, optionally wherein the electrically conductive material comprises electrically conductive particles, and a second compartment comprising a second collagen scaffold, and wherein the second compartment is disposed on the first compartment and the pores are longitudinally aligned between the compartments.

In some embodiments, the scaffold comprising collagen, the first collagen scaffold and/or the second collagen scaffold comprises collagen-glycosaminoglycan (CG). In some embodiments, a collagen concentration of the first collagen scaffold varies as compared to a collagen concentration of the second collagen scaffold. In some embodiments, the collagen concentration for the first collagen scaffold ranges from about 0.5 weight percent (wt %) to about 1.5 wt % and the collagen concentration for the second collagen ranges from about 1.5 wt % to about 5 wt %.

In some embodiments, the electrically conductive particles are microparticles. In some embodiments, the electrically conductive material comprises electrically conductive polypyrrole.

In some embodiments, a method for making a multicompartment conductive collagen scaffold composite is provided. In some embodiments, the method comprises providing a scaffold comprising collagen; contacting the scaffold comprising collagen with an electrically conductive material, optionally wherein the electrically conductive material comprises electrically conductive particles, to form a composite comprising the scaffold comprising collagen and the electrically conductive particles; and freeze-drying the composite to form a multicompartment conductive collagen scaffold composite comprising longitudinally aligned pores. In some embodiments, the method comprises providing a first compartment comprising a first collagen scaffold and an electrically conductive material, optionally wherein the electrically conductive material comprises electrically conductive particles, and a second compartment comprising a second collagen scaffold; contacting the first compartment and the second compartment to form a composite comprising the first compartment and the second compartment; and freeze-drying the composite to form a multicompartment conductive collagen scaffold composite comprising longitudinally aligned pores.

In some embodiments, the scaffold comprising collagen, the first collagen scaffold and/or the second collagen scaffold comprises collagen-glycosaminoglycan (CG). In some embodiments, a collagen concentration of the first collagen scaffold varies as compared to a collagen concentration of the second scaffold. In some embodiments, the collagen concentration for the first collagen scaffold ranges from about 0.5 wt % to about 1.5 wt % and the collagen concentration for the second collagen scaffold ranges from about 1.5 wt % to about 5 wt %.

In some embodiments, the electrically conductive particles are microparticles. In some embodiments, the electrically conductive material comprises electrically conductive polypyrrole. In some embodiments, contacting the scaffold comprising collagen with the electrically conductive material, optionally wherein the electrically conductive material comprises electrically conductive particles, comprises layering or coating the scaffold comprising collagen with the electrically conductive particles. In some embodiments, contacting the first compartment and the second compartment to form a composite comprising the first compartment and the second compartment comprises layering two different suspensions on top of one another, wherein a first suspension of the two different suspensions comprises a first collagen scaffold and an electrically conductive material, optionally wherein the electrically conductive material comprises electrically conductive particles, and a second suspension of the two different suspension comprises a second collagen scaffold. In some embodiments, the method comprises allowing the suspensions to interdiffuse prior to freeze-drying.

In some embodiments, wherein the first and second suspensions are prepared by mixing type I collagen, chondroitin sulfate, and acetic acid. In some embodiments, the freeze drying comprises directional lyophilization.

In some embodiments, the presently disclosed subject matter provides a method of treating a skeletal muscle injury in a subject in need thereof. In some embodiments, the method comprises providing a multicompartment conductive collagen scaffold composite, comprising a scaffold comprising collagen and an electrically conductive material, optionally wherein the electrically conductive material comprises electrically conductive particles, and further comprising longitudinally aligned pores; and implanting the multicompartment conductive collagen scaffold composite at a site of the skeletal muscle injury in the subject. In some embodiments, the multicompartment conductive collagen scaffold composite further comprises a first compartment comprising a first collagen scaffold and an electrically conductive material, optionally wherein the electrically conductive material comprises electrically conductive particles, and a second compartment comprising a second collagen scaffold, and wherein the second compartment is disposed on the first compartment and the pores are longitudinally aligned between the compartments

In some embodiments, the scaffold comprising collagen, the first collagen scaffold and/or the second collagen scaffold comprises collagen-glycosaminoglycan (CG). In some embodiments, a collagen concentration of the first collagen scaffold varies as compared to a collagen concentration of the second scaffold. In some embodiments, the collagen concentration for the first collagen scaffold ranges from about 0.5 wt % to about 1.5 wt % and the collagen concentration for the second collagen scaffold ranges from about 1.5 wt % to about 5 wt %.

In some embodiments, the electrically conductive particles are microparticles. In some embodiments, the electrically conductive material comprise electrically conductive polypyrrole. In some embodiments, the skeletal muscle injury comprises an injury at a muscle-tendon junction (MTJ).

In some embodiments, the interdiffusion time ranges from about 15 minutes to 60 minutes, include about 30 or about 45 minutes as particular examples. In some embodiments, pore sizes range from about 50 μm to about 250 μm in diameter, including about 150 μm and about 200 μm as particular examples. In some embodiments, the pores are elongated. In some embodiments, the conductive material (e.g., particle) content ranges from about 0.1 wt % to about 3 wt %.

In some embodiments, cells are seeded to the composite. In some embodiments, seeding the cells comprises adding a cell solution directly to the compartments. Thus, in some embodiments, one or more compartments are deployed either with or without seeded cells. In some embodiments, the seeding cells can comprise muscle-derived cells (including myoblasts and satellite cells), fibroblasts, and neural cells (neural stem cells, motor neurons), and combinations thereof. Other particular examples of seeding cells are disclosed elsewhere herein.

Accordingly, it is an object of the presently disclosed subject matter to provide multicompartment conductive collagen scaffold composite compositions, and method of making and using the same. An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIG. 1: Musculotendinous junction (MTJ) structure. Schematic of MTJ highlighting key features (left) and electron micrograph showing common injury site near the MTJ (right). Scale bar: 2 μm. Figure adapted from Yang and Temenoff, 2009 (left) and Tidball et al., 1993 (right).

FIG. 2: Conductive CG scaffolds have aligned pores. SEM of conductive CG scaffold longitudinal plane highlights structural alignment mimicking muscle. Scale bar: 1 mm.

FIG. 3: MDC characterization. MDCs represent a mixed population of cells as shown by Panel A) Pax7, Panel B) MyoD, and Panel C) desmin staining. Scale bars: 50 μm. Panel D) Bar graph showing quantification of MDC fraction expressing Pax7, MyoD, and desmin respectively. Figure reproduced from Corona et al., 2012.

FIG. 4: Bar graph showing polypyrrole (PPy) incorporation increases scaffold conductivity. Chronopotentiometry measurements of CG scaffold conductivity as a function of increasing PPy content. **: P<0.01.

FIG. 5: Bar graphs showing myoblasts grow in conductive CG scaffolds. Myoblasts show sustained metabolic activity over 7 days of culture. *: P<0.05. Legend top to bottom corresponds to bars left to right.

FIG. 6: Images (left) and graph (right) showing myoblasts conform to scaffold structural cues. Cells align in the longitudinal plane as measured by F-actin orientation. Scale bar: 100 μm. When the Figure is presented in color, the following color scheme is used: Blue: nuclei, Green: F-actin, Red: collagen.

FIG. 7: Images (upper) and bar graphs (lower) showing conductive CG scaffolds support early myogenesis. When the Figure is presented in color, the following color scheme is used: Blue: nuclei, Green: myosin heavy chain (MHC), Red: collagen. Scale bar: 100 μm. *: P<0.05.

FIG. 8: Bioreactor conditioning guides cell organization. Left: photo of bioreactor design. Right: Muscle progenitor cells grown on ECM scaffolds with bioreactor stimulation show greater alignment and organization as measured by actin and myosin expression. Figure modified from Moon et al., 2008.

FIG. 9: Schematic of electromechanical bioreactor stimulation. Mechanical stimulation: 10% strain, 3 stretches/min for the first 5 min of each hour. Electrical stimulation: biphasic rectangular pulses, 2 ms, 20 Hz, 2.5 V/mm. 3 pulses/min for the first 5 min of each hour.

FIG. 10: Images (left) and graph (right) showing in vivo force generation testing. Testing is well tolerated at multiple time points and enables repeated quantification of isometric torque over a range of testing frequencies. Figure modified from Corona et al., 2013 and Mintz et al., 2016.

FIG. 11: Heatmap showing plasma cytokine quantification. Plasma samples taken shortly following surgery underwent multiplexed quantitative analysis of 27 inflammatory cytokines to track early response to biomaterial implantation.

FIGS. 12A-12C: Multi-compartment scaffolds show graded presentation of conductive PPy. FIG. 12A) Photo of multi-compartment scaffolds shows clear delineation between ‘tendon’ (CG, white) and ‘muscle’ (CG-PPy, black) regions. FIG. 12B) Energy-dispersive spectroscopy (EDS) characterization shows differences in Cl content (PPy dopant) between ‘tendon’ and ‘muscle’ compartments with an interface width of ˜400 μm. FIG. 12C) EDS mapping of Cl content (referred to generally with white arrowheads and indicated in magenta when shown in color) overlaid on SEM images of distinct scaffold regions. Scale bar: 500 μm.

FIG. 13: Plot (left) and images (right) showing multi-compartment scaffolds display large open pore structure. Scaffold pore size quantification from confocal images show average pore diameters >150 μm in both compartments. Scale bar: 100 μm. When shown in color, collagen is shown in red.

FIGS. 14A-14D: Schematic of VML/MTJ model. Schematic shows FIG. 14A) lateral view of the rat hindlimb, where the tibialis anterior (TA) muscle is highlighted in red (when shown color) and the tendon is shown by an arrowhead to the left of the muscle and in blue (when in color). FIG. 14B) The model is comprised of the removal of the middle ˜50% of the tendon and ˜20% of the TA muscle, including the MTJ. FIG. 14C) The scaffold is then implanted in the defect site. FIG. 14D, photograph of model.

FIG. 15: Motion capture and biomechanical modeling of rodent gait following TA VML injury. Left: 3D overlay of motion capture markers placed on spine, hip, knee, ankle, and toe. Right: OpenSim model reconstruction of rat hindlimb showing markers. Figure modified from Dienes et al., 2019.

FIG. 16: Images showing histological and immunohistochemical analysis of TA muscle repair. Representative histological samples (Masson's Trichrome) show that bioreactor conditioning supports reduced fibrosis and increased expression of neurofilament 200 (NF200). Scale bars: 250 μm (left), 100 μm and 25 μm (right). Figure modified from Mintz et al., 2019.

FIG. 17: Schematic of collagen-glycosaminoglycan-polypyrrole (CG-PPy) scaffold fabrication. A suspension of type I collagen, chondroitin sulfate, and acetic acid was prepared using a high shear homogenizer in a recirculating chiller to prevent collagen denaturization. Synthesized PPy particles were then added to the slurry and homogenized by vortexing. Aligned scaffolds were produced via directional lyophilization using a custom designed thermally mismatched mold.

FIG. 18: Scaffolds show longitudinally aligned pore orientation independent of PPy content. SEM images (Panels A-D) show isotropic pore structure along the scaffold transverse plane and highly aligned, elongated pores along the scaffold longitudinal plane (Panels E-H) mimicking native skeletal muscle tissue organization. Panel I) Histogram of scaffold strut orientation angles within the transverse (open circles) and longitudinal (closed circles) planes of the scaffolds. Scale bar: 1 mm. n=3 scaffolds per experimental group.

FIGS. 19A to 19E: Polypyrrole (PPy) content is uniformly distributed throughout scaffolds. FIGS. 19A-19D) Energy-dispersive X-ray spectroscopy (EDS) maps of Cl content overlaid on SEM images of longitudinal scaffold planes show increasing Cl content with increasing PPy content as indicated by the arrowheads in FIGS. 19A-19D and pink pixels when shown in color. Additionally, all maps show uniform Cl distribution and thus PPy particle homogeneity within CG-PPy scaffolds. FIG. 19E) EDS spectra of the CG-PPy scaffolds show elemental distributions across experimental groups, including increasing Cl peak intensity with increasing PPy content. Scale bar: 500 μm. Legend features lead lines to graphs and includes 0% PPY (light blue when shown in color), 0.1% PPY (dark blue when shown in color), 0.2% PPY (purple when shown in color), and 0.5% PPY (black when shown in color).

FIG. 20. Scaffold pore architecture is not affected by PPy incorporation. Pore microstructure was assessed using MATLAB analysis of confocal images of scaffold collagen backbone. Directional heat transfer during lyophilization resulted in scaffolds containing a 3D pore microstructure with an average pore size of 150±26 μm in the transverse plane (Panels A-D) and 203±44 μm in the longitudinal plane (Panels E-H). Insets: best fit ellipse representations of pore shape generated from MATLAB analysis. Panel I) Box plot of transverse and longitudinal scaffold pore diameters. Data presented for each group includes the mean (x) and median (bar). Whiskers represent the 1^{st} and 4^{th} quartiles (or 1.5 times the interquartile range with data points outside this range shown individually) while boxes represent the 2^{nd} and 3^{rd} quartiles. Scale bar: 100 μm. n=15 scaffolds per experimental group.

FIG. 21. PPy incorporation leads to significantly increased scaffold conductivity. Panel A) Schematic illustrating chronopotentiometry experimental setup. Panel B) The conductivity of the CG-PPy scaffolds was analyzed using chronopotentiometry and indicated that scaffolds with 0.5 wt % polypyrrole content had ˜5-fold higher conductivity than non PPy-containing control scaffolds. **: P<0.01. n=3 scaffolds per experimental group.

FIG. 22. Bar graphs showing CG-PPy scaffolds support sustained and increasing myoblast metabolic activity. Quantification of metabolic activity over 7 days of culture showed that scaffolds with polypyrrole content ranging from 0-0.5 wt % all supported sustained, increasing metabolic activity. *: P<0.05. n=4 scaffolds per experimental group. Legend top to bottom corresponds to bars left to right.

FIG. 23. Aligned scaffolds guide 3D myoblast cytoskeletal alignment. Confocal imaging showed that cells spread within CG scaffolds and conformed to contact guidance cues presented by the scaffold microstructure. Cells displayed more random, isotropic organization in the scaffold transverse plane (Panels A-B) and were organized and highly aligned in the scaffold longitudinal plane (Panels C-D). Panel E) Histogram indicating F-actin cytoskeletal anisotropic alignment in the longitudinal (solid lines) vs. transverse (dashed lines) planes. Scale bar: 100 μm. n=3 scaffolds per experimental group. When the Figure is presented in color, the following color scheme is used: Blue: nuclei, Green: F-actin, Red: collagen (e.g, in Panel A).

FIG. 24. CG-PPy scaffolds support improved early myoblast differentiation. C2C12 myoblasts cultured within CG scaffolds containing Panels A) 0% or Panels B) 0.5% PPy express myosin heavy chain (MHC) after 5 days of culture in myogenic differentiation media (images from transverse plane). Panel C) PPy-containing scaffolds showed increased fraction of MHC-positive cells after both 2 and 5 days in differentiation media. Panel D) The number of mononucleated MHC-positive myotubes was significantly reduced in PPy-containing scaffolds with higher fractions of multinucleated myotubes observed. Panel E) OrientationJ analysis of myotube alignment indicated preferential alignment in the anisotropic longitudinal plane (solid lines) compared to the isotropic transverse plane (dashed lines). Scale bar: 100 μm. *: P<0.05, * *: P<0.01. n=4 scaffolds per experimental group (panels C, D), n=3 scaffolds per experimental group (panel E). When the Figure is presented in color, the following color scheme is used: Blue: nuclei, Green: MHC, Red: collagen.

FIG. 25. (Upper Left Panel) Polypyrrole (PPy) nanoparticles were synthesized via an oxidation reaction. FTIR analysis showed peaks at 1580 cm⁻¹ associated with C═C stretching and peaks at 1350 cm⁻¹ and 1220 cm⁻¹ that are indicative of C—H wagging vibrations and conjugated C—N in-plane stretching. (Upper Center and Upper Right Panel) Particle size analysis using scanning electron microscope (SEM) images indicated the production of homogeneous particles with an average diameter of 527.1±96.7 nm. n=230 PPy particles. (Bottom Panels) PPy nanoparticles were incorporated into a collagen chondroitin sulfate suspension prior to lyophilization. SEM images of lyophilized scaffolds show uniform distribution of PPy particles (arrows, presented in red when in color) throughout the scaffold with minimal particle aggregation.

FIG. 26. Bar graphs shown quantification of metabolic activity over 7 days of culture showed that scaffolds with higher loading of polypyrrole (1.5, 3 wt %) promoted significantly decreased C2C12 myoblast metabolic activity. *: P<0.05, ***: P<0.001, ****: P<0.0001. n=4 scaffold per experimental group. Legend left to right corresponds to bars left to right.

FIG. 27. Bar graph showing the percentage of the image area occupied by myosin heavy chain (MHC) staining was quantified using ImageJ. After 5 days in differentiation media there was significantly higher signal in the PPy-containing group compared to day 2 signal for the CG-only scaffold control. *: P<0.05. n=4 scaffolds per experimental group.

FIG. 28. Bar graph showing myotube diameter was quantified using DiameterJ, an ImageJ plugin. There were no significant changes observed in myotube diameter as a function of culture time or PPy incorporation. n=3 scaffolds per experimental group.

FIG. 29. Bar graph (right) and images (left) showing assessment of myogenic differentiation indicated that culture time and PPy incorporation did not appreciably affect MyoD expression. Scale bar: 100 μm. n=3 scaffolds per experimental group.

FIG. 30. Expression of the left panel) Myh2 and right panel) Myod1 genes encoding for MHC and MyoD respectively were quantified after 2 and 5 days of culture in differentiation media for the 0 and 0.5 wt % PPy scaffolds. No statistically significant differences were found between any of the experimental groups. Data are expressed as the mean fold change normalized to expression in the 0% PPy day 2 group, n=3 scaffolds per experimental group.

FIG. 31. Schematic of cellular microenvironment (Gattazzo et al., 2014) and skeletal muscle organization (Gilles & Lieber, 2011).

FIG. 32. Schematic showing suspension of type I collagen, chondroitin sulfate, and acetic acid was prepared using a high shear homogenizer in a recirculating chiller to prevent collagen denaturization. Conductive PPy particles were synthesized via an oxidation reaction with FeCl3 and vortexed into the suspension. The suspension was degassed to remove bubbles from the slurry FIG. 33. Schematic of multi-compartment scaffold fabrication. A 2.5 wt % CG suspension (‘tendon’ compartment) was added atop a 0.5 wt % PPy-doped CG suspension (‘muscle’ compartment) and allowed to interfuse for 30 min. The slurry was then directionally freeze dried using a thermally-mismatched mold to recreate the graded interface of the MTJ.

FIGS. 34, 35 and 36. Graph, Histogram and Images, respectively, showing orientation analysis shows isotropic pore structure along the transverse plane and highly aligned, elongated pores, along the longitudinal plane mimicking native skeletal muscle and tendon tissue architecture. Scale bar: 1 mm.

FIG. 37. Schematic of the VML injury model to the middle third of the left TA. VML injuries were reproducibly created by surgically resecting ˜20% of the TA muscle weight. CG scaffolds were able to fill the VML defect, conform to the injury dimensions, and remain in place following suturing.

FIG. 38. Animal body weight showed a healthy weight gain in all treatment groups over the course of 12 weeks that was not statistically different between groups at any time point (two-way ANOVA, P>0.05). Creation of the VML defect was reproducible across all experimental groups with no significant differences observed (one-way ANOVA, P>0.05). Image of in vivo functional testing of the TA following muscle VML injury in which the left leg of an anesthetized rat was attached to a force plate and repeatedly stimulated using electrodes placed along the peroneal nerve. When the Figure is presented in color, the following color scheme is used: Gray: no repair (NR), Blue: PPy, Red: CG. Lead lines provided from legend in box graph to boxes and to lines in line graph. The following legend is also used circles, NR; squares, CG; and triangles, PPy.

FIG. 39. Line graphs and bar graphs showing functional recovery of TA muscles was measured by isometric muscle contraction (see FIG. 34). Baseline isometric torque measurements were not significantly different between groups but displayed a marked reduction in force production 4 weeks post injury. At 8 weeks non-conductive CG scaffolds showed an increase in torque production at higher stimulation frequencies compared to no repair (NR) muscles. At 12 weeks post VML both PPy-doped and non-conductive CG scaffolds showed an increase in isometric torque compared to no repair (NR) muscles. Statistically significant differences from no repair are denoted by g (CG) and p (PPy). When isometric torque was normalized to baseline values PPy-doped and non-conductive CG scaffolds supported improved functional recovery compared to non-treated muscles. Data presented as Mean±SD. When the Figure is presented in color, the following color scheme is used: Gray: NR, Blue: PPy, Red: CG. Lines in line graphs also labelled with NR, PPy, and CG. following legend is also used circles, NR; squares, CG; and triangles, PPy.

### DETAILED DESCRIPTION

Skeletal muscle is characterized by its three-dimensional (3D) anisotropic architecture comprising highly aligned and electrically-excitable muscle fibers that enable normal movement. Currently available biomaterial-based tissue engineering approaches to repair skeletal muscle are limited due to difficulties combining 3D structural alignment (to guide cell/matrix organization) and electrical conductivity (to enable electrically-excitable myotube assembly and maturation). The presently disclosed subject matter produced aligned and electrically conductive 3D collagen scaffolds using a freeze-drying approach. In representative, non-limiting embodiments, conductive polypyrrole (PPy) nanoparticles were synthesized and directly mixed into a suspension of type I collagen and chondroitin sulfate followed by directional lyophilization. Scanning electron microscopy (SEM), energy-dispersive spectroscopy (EDS), and confocal microscopy showed that directional solidification resulted in scaffolds with longitudinally aligned pores with homogeneously-distributed PPy content. Chronopotentiometry verified that PPy incorporation resulted in a five-fold increase in conductivity compared to non-PPy-containing collagen scaffolds without detrimentally affecting myoblast metabolic activity. Furthermore, the aligned scaffold microstructure provided contact guidance cues that directed myoblast growth and organization. Incorporation of PPy also promoted enhanced myotube formation and maturation as measured by myosin heavy chain (MHC) expression and number of nuclei per myotube. Together these data suggest that aligned and electrically conductive 3D collagen scaffolds are useful for skeletal muscle tissue engineering.

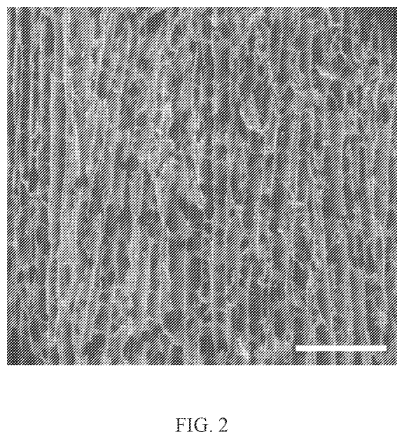

Fully functional muscle repair requires both innervation of the regenerating muscle tissue to enable contractile force generation and restoration of the intact MTJ to facilitate force transmission to the skeletal system and normal locomotion. Therefore, an aspect of the presently disclosed subject matter is to apply a tissue engineering scaffold mimicking the structure of the MTJ to promote innervated functional regeneration of VML/MTJ injuries. The presently disclosed subject matter provides an innovative biomaterials-based approach that in some embodiments employs a 3D aligned and electrically conductive collagen-glycosaminoglycan (CG) scaffold (see, e.g. FIG. 2) that recapitulates both the anisotropic extracellular matrix (ECM) organization and electrical excitability of native skeletal muscle (Basurto et al., 2020). Additional aspects include employing heterogeneous biomaterial design (Martin et al., 2011; Caliari et al., 2015a; Caliari et al., 2015b; Hui et al., 2019), bioreactor conditioning for muscle tissue engineering (Moon et al., 2008; Machingal et al., 2011; Corona et al., 2013), and small animal VML models (Corona et al., 2012; Corona et al., 2013; Passipieri et al., 2017; Baker et al., 2017; Passipieri et al., 2019). Thus, in some embodiments, the presently disclosed subject matter provides an engineered biomaterial with spatially-defined microenvironmental cues paired with bioreactor preconditioning of myogenic and neuronal cells to further provide regeneration of clinically relevant VML/MTJ injuries.

In some embodiments, the presently disclosed subject matter evaluates the combined ability of 3D scaffold alignment and electrical conductivity to drive in vitro myogenesis of muscle-derived cell (MDC) and neural stem cell (NSC) co-cultures. Recent work has demonstrated the utility of co-culturing neural and muscle progenitor cells to drive improved in vitro myogenesis (Kim et al., 2020). However, it is unknown if biomimetic scaffold cues, including 3D structural alignment and electrical conductivity, can further amplify this process. The presently disclosed subject matter evaluates MDC and NSC viability, proliferation, cytoskeletal organization, and myotube and neuromuscular junction (NMJ) formation within both conductive and non-conductive aligned CG scaffolds using confocal microscopy and immunocytochemistry. Antibody-based imaging of myosin heavy chain (MHC), class III β-tubulin (βIIIT), and acetylcholine receptor (AChR) are used to evaluate myotube formation, neuronal differentiation, and NMJ development respectively. Bioreactor-conditioned tissue engineering scaffolds (Moon et al., 2008; Machingal et al., 2011; Corona et al., 2013) are provided to interrogate the potential of electrical and/or mechanical stimulation to further improve MDC/NSC differentiation and maturation.

The presently disclosed subject matter also evaluates the ability of 3D multi-compartment scaffolds with co-cultured MDCs and NSCs to guide repair of musculotendinous junction (MTJ) volumetric muscle loss (VML) injuries. CG scaffolds are promising biomaterials for tissue engineering due to their ability to suppress scarring and promote functional regeneration of tissues including dermis (Yannas et al., 1989), peripheral nerve (Harley et al., 2004), and osteochondral tissue (Getgood et al., 2012). In accordance with the presently disclosed subject matter, graded, anisotropic CG scaffolds with spatially-defined electrical conductivity and mechanics to recapitulate the biophysical properties of the MTJ are provided. The capability of this scaffold, with or without bioreactor-conditioned MDCs and NSCs, is assessed to support functional repair of rat tibialis anterior VML/MTJ defects as a model, non-limiting system. Functional recovery is quantified through repeated in vivo measurements of force generation and gait biomechanics over a pre-determined period, such as 24 weeks. Tissue repair quality is measured through histological and immunohistochemical analysis of muscle fiber organization, NMJ assembly, MTJ marker expression, vascularization, and fibrosis.

The presently disclosed subject matter can be used to treat VML defects, particularly those involving nerve and/or MTJ damage, which are devastating to patients and notoriously difficult to repair. The presently disclosed subject matter addresses previously intractable biological and clinically relevant questions, including understanding of how structural and electrical signals can synergistically promote functional myogenesis in biomimetic tissue engineering scaffolds. Overall, the presently disclosed subject matter establishes an innovative paradigm for regenerating multi-tissue interfaces and innervating electrically-responsive tissue and provides for the repair of complex VML injuries, especially those involving damage to surrounding tissues such as tendon and peripheral nerves, by restoring contractile force generation and movement abilities.

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. DEFINITIONS

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Unless otherwise indicated, all numbers expressing quantities of size, temperature, time, weight, volume, concentration, capacitance, specific capacity, discharge capacity, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±20% or ±10%, in another example ±5%, in another example ±1%, and in still another example ±0.1% from the specified amount, as such variations are appropriate to perform the disclosed methods. Thus, the term “about,” as used herein, means approximately, in the region of, roughly, or around. When the term “about” is used in conjunction with a numerical range, it modifies that range by extending the boundaries above and below the numerical values set forth. In one aspect, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that the presently disclosed composites and related components discussed herein may take on all shapes along the entire continual geometric spectrum of manipulation of x, y and z planes to provide and meet the environmental, anatomical, and structural demands and operational requirements. Moreover, locations and alignments of the various components may vary as desired or required. Also, references to “first”, “second”, and “additional” are not meant to be limiting but rather used to enhance clarity of description.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the presently disclosed subject matter may constitute various sizes, dimensions, contours, rigidity, shapes, flexibility and materials as it pertains to the components or portions of components of the presently disclosed subject matter, and therefore may be varied and utilized as desired or required.

Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

Additional descriptions of aspects of the present disclosure will now be provided with reference to the accompanying drawings. The drawings form a part hereof and show, by way of illustration, specific embodiments or examples.

### II. GENERAL CONSIDERATIONS

The presently disclosed subject matter addresses the clinically relevant problem of treating volumetric muscle loss (VML) injuries, particularly those with damage to neighboring connective and nervous tissue. In some embodiments, biomaterial scaffolds mimicking the anisotropic collagenous architecture and electrical conductivity of skeletal muscle are provided to solve this challenge.

VML impact on human health. Skeletal muscle injuries are pervasively common in patients ranging from elite athletes and soldiers to the elderly. While the battle mortality rate has fallen from 30% in World War II to less than 10% in Iraq and Afghanistan (Goldberg, 2010) due to advances in medical technologies, injuries to the extremities have been the most common wounds sustained with 92% of the injured warfighters with muscle deformities identified as VML patients (Corona et al., 2015). VML is defined as extensive muscle damage that leads to a permanent loss of muscle mass and function (Machingal et al., 2011; Corona et al., 2012). Unfortunately, strategies for treating VML injuries are relatively understudied compared to bone injuries even though 82% of combat-related fractures are open (i.e., involve open skin wounds and surrounding tissue damage; Owens et al., 2007). Further, the average lifetime disability payout per VML patient is on the order $400,000 (Corona et al., 2015). Together, these data highlight the pressing need for better therapies to address complex VML injuries.

Standard of care for VML. Current treatments for VML injuries include autologous tissue transfer, or surgical installation of muscle flaps, followed by physical therapy (Corona et al., 2015; Sarrafian et al., 2018). Unfortunately, these methods are expensive, time-intensive, and typically result in poor functional improvement as well as donor site morbidity. Moreover, for large wounds, there is often not enough donor tissue to make viable autografts. Nonsurgical options include bracing (Grogan & Hsu, 2011), but are limited to VML injuries below the knee. In either case the ability of patients to recover functionality, defined as the ability to produce muscle-generated force and execute normal movements, leaves much to be desired. Additional complications arise from the considerable variability among VML injuries in size, location, and heterogeneity of affected tissues. Emergent approaches use cell-seeded extracellular matrix (ECM) scaffolds to drive VML repair (Brown et al., 2009; Machingal et al., 2011; Corona et al., 2012; Turner & Badylak, 2012; Saclier et al., 2013). However, a 2018 review of 40 clinical studies concluded that current ECM scaffolds fail to provide ‘substantive clinical benefit’ to patients (Sarrafian et al., 2018). Given these myriad challenges, there is a significant opportunity for innovative tissue engineering approaches to solve this clinical problem.

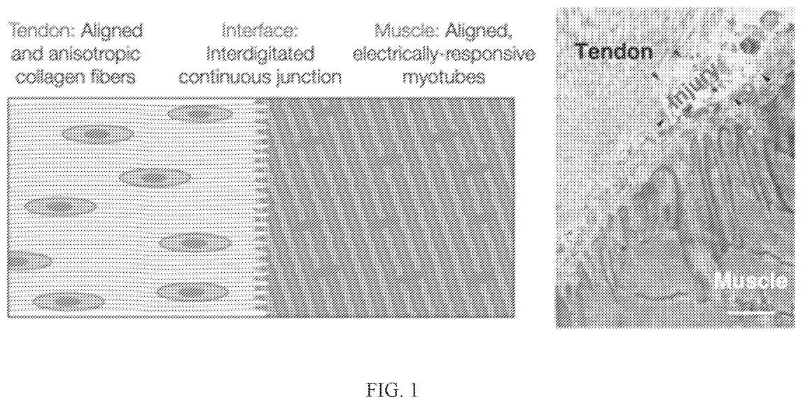

Repairing connective and nervous tissue damage in VML. In addition to damaged muscle, VML injuries are often accompanied by damage to surrounding tissue including skin, vasculature, peripheral nerves, and tendon. As with other orthopedic interfaces, the muscle-tendon (musculotendinous) junction (MTJ) is a common injury site (FIG. 1). Proper MTJ function is critical for normal movement as muscle-generated force is transmitted via tendons to the skeletal system. The MTJ is characterized by folding of tendon collagen fibers into ‘finger-like’ processes generated by myocytes, permitting transmission of muscle-generated forces to tendon. Together, this multi-modal folding integration of muscle and tendon significantly increases the surface area at the MTJ (Trotter, 2002), reducing stress concentrations during muscle contraction and subsequent tendon loading (Gross & Hoffmann, 2013). Despite this, the MTJ is generally considered the weakest part of the muscle, making it an important tissue engineering target.

Efficient muscle force generation also requires functional innervation. Peripheral nerve damage is a significant issue affecting approximately 25% of VML patients (Beltran et al., 2012). These injuries can result in partial or complete nerve rupture, leading to a loss of sensory and motor function as well as muscle denervation and atrophy due to the slow rate of repair (Menorca et al., 2013). While previous work has outlined certain benefits of using co-cultures of neurons and myogenic cells to improve muscle innervation through establishment of neuromuscular junctions (NMJs; Rao et al., 2018; Kim et al., 2020), innervation of the large three-dimensional (3D) muscle defects created from VML remain challenging to treat. Therefore, in some embodiments, the presently disclosed subject matter combines instructive 3D biomaterial scaffolds with co-cultured neuronal and myogenic cells to drive both muscle innervation (to enable force generation) and muscle integration with the skeletal system via the MTJ (to enable normal movement).

Biomimetic design criteria for guiding muscle repair. Skeletal muscle is characterized by its highly organized structure in which aligned myofibers and larger muscle fiber bundles (Musumeci et al., 2015; Nishimura, 2015) are surrounded by a collagenous ECM (Gillies & Lieber, 2011). Muscle fibers and ECM in turn interdigitate with the fibrillar collagen of tendon to from a contiguous interface at the MTJ (Charvet et al., 2012; Tadros et al., 2018; Skalec et al., 2019). It is this anisotropic organization, from the molecular to tissue scale, that ultimately allows effective force transfer during muscle contraction. In addition to the highly organized structure of skeletal muscle, bioelectrical stimuli are known to regulate myoblast differentiation and tissue regeneration (Weiss et al., 1990; Mihic et al., 2015; Yang et al., 2016a; McLaughlin & Levin, 2018; Alegret et al., 2019; Fennelly & Soker, 2019). While peripheral nerves connect with skeletal muscle through NMJs to generate muscle contractions, following VML the ECM becomes disorganized and muscle-nerve connections are often disrupted, resulting in permanent loss of muscle function (Bursac et al., 2015). This motivates the use of regenerative templates that direct anisotropic cell and ECM organization while also providing electrically conductive cues to facilitate innervation and myogenesis following injury.

Biomaterial-based strategies for complex VML injuries: A recent study found that incorporation of neural stem cells (NSCs) with muscle progenitor cells into a bioprinted hydrogel accelerated functional regeneration in a rat VML model (Kim et al., 2020). The inclusion of NSCs supported improved myofiber and NMJ formation in vitro, underscoring the utility of neuronal co-culture for muscle repair. However, despite the electrically responsive nature of skeletal muscle and peripheral nerves, most 3D biomaterial scaffolds have not included electrically conductive groups. Polypyrrole (PPy) in particular has proven to be a promising conductive material for electrically-responsive tissue repair (Schmidt et al., 1997; Elias et al., 2019; Sun et al., 2019). However, due to difficulties in processing, including poor solubility and mechanical brittleness, the majority of conductive biomaterials present cells with a 2D environment that does not accurately recapitulate the complexity and heterogeneity of their native 3D environments (Balint et al., 2014; Alegret et al., 2019). Furthermore, conductive polymers alone do not possess the anisotropy and cell adhesive sites that are necessary for skeletal muscle repair. This difficulty in processing conductive materials into 3D aligned structures needed for musculoskeletal tissue engineering has proven to be a limitation in the field. In some embodiments, the presently disclosed subject matter provides the use of hybrid materials comprising conductive polymers and anisotropic cell-instructive biomaterials to leverage the strengths of both components.

Biomaterial-based tissue engineering approaches for MTJ repair have also been limited. Larkin et al. used an anchored gel system to promote contraction-mediated formation of aligned myotendinous constructs (Larkin et al., 2006). More recently, one of the first biomaterials engineered specifically for MTJ regeneration was fabricated by co-electrospinning PCL/collagen and PLLA/collagen nanofibers to create two mechanically distinct regions corresponding to muscle and tendon respectively along with an overlap region representing the junction (Ladd et al., 2011). While these materials represent exciting steps forward, neither has been validated in vivo.

### III. REPRESENTATIVE EMBODIMENTS

An aspect of an embodiment of the presently disclosed subject matter provides, among other things, a multicompartment conductive collagen scaffold and method of making and using the same. Another aspect of an embodiment of the presently disclosed subject is a compartment comprising a conductive collagen scaffold and a method of making and using the same.

Skeletal muscle injuries are often accompanied by damage to surrounding tissue including skin, vasculature, peripheral nerves, and tendon. As with other orthopedic interfaces, the muscle-tendon junction (MTJ) is a common injury site. Proper MTJ function is critical for normal movement as muscle-generated force is transmitted via tendons to the skeletal system. Unfortunately, the MTJ is generally considered the weakest part of the muscle, making it an important tissue engineering target. To address this clinical need, an aspect of an embodiment of the presently disclosed subject matter provides, among other things, a multicompartment conductive collagen scaffold mimicking the gradations in electrical conductivity and mechanical properties found in the anisotropic MTJ. In a representative, non-limiting embodiment, a collagen-glycosaminoglycan (CG) scaffold base was chosen in part for its rich history of use in a range of tissue engineering applications including bone and tendon as well as clinically for peripheral nerve and skin regeneration. CG scaffolds block wound contraction and limit myofibroblast activation, leading to reduced scarring and improved wound healing. CG scaffolds are typically fabricated by freeze-drying where ice crystals template the resultant 3D interconnected macropore structure. In some embodiments, a directional freeze-drying approach was employed to form longitudinally aligned, elongated pores mimicking native tissue organization. In some embodiments, two different suspensions were layered on top of one another; one containing high CG content to mimic the more mechanically robust tendon, and one containing electrically conductive polypyrrole microparticles. These suspensions were allowed to interdiffuse prior to freeze-drying to create layered multicompartment composite materials for MTJ tissue engineering.

An aspect of an embodiment of the presently disclosed subject matter provides a system and method for, among other things, developing graded, anisotropic scaffolds with spatially-defined biophysical properties to mimic the MTJ interface.

An aspect of an embodiment of the presently disclosed subject matter provides a system and method for, among other things, establishing a VML/MTJ injury model in the tibialis anterior (TA) of male Lewis Rats to evaluate functional recovery.

In some embodiments, the presently disclosed subject matter provides a compartment comprising a conductive collagen scaffold. In some embodiments, the presently disclosed subject matter provides a multicompartment conductive collagen scaffold composite.

The presently disclosed subject matter provides in some embodiments a multicompartment conductive collagen scaffold composite. In some embodiments, the multicompartment conductive collagen scaffold composite comprises a scaffold comprising collagen and an electrically conductive material, optionally wherein the electrically conductive material comprises electrically conductive particles, and further comprising longitudinally aligned pores.

In some embodiments, the multicompartment conductive collagen scaffold composite further comprises a first compartment comprising a first collagen scaffold and an electrically conductive material, optionally wherein the electrically conductive material comprises electrically conductive particles, and a second compartment comprising a second collagen scaffold, and wherein the second compartment is disposed on the first compartment and the pores are longitudinally aligned between the compartments.

In some embodiments, the scaffold comprising collagen, the first collagen scaffold and/or the second collagen scaffold comprises collagen-glycosaminoglycan (CG). In some embodiments, a collagen concentration of the first collagen scaffold varies as compared to a collagen concentration of the second collagen scaffold. In some embodiments, the collagen concentration for the first collagen scaffold ranges from about 0.5 weight percent (wt %) to about 1.5 wt % and the collagen concentration for the second collagen ranges from about 1.5 wt % to about 5 wt %.

In some embodiments, the electrically conductive particles are microparticles. In some embodiments, the electrically conductive material comprises electrically conductive polypyrrole.

In some embodiments, a method for making a multicompartment conductive collagen scaffold composite is provided. In some embodiments, the method comprises providing a scaffold comprising collagen; contacting the scaffold comprising collagen with an electrically conductive material, optionally wherein the electrically conductive material comprises electrically conductive particles, to form a composite comprising the scaffold comprising collagen and the electrically conductive particles; and freeze-drying the composite to form a multicompartment conductive collagen scaffold composite comprising longitudinally aligned pores. In some embodiments, the method comprises providing a first compartment comprising a first collagen scaffold and an electrically conductive material, optionally wherein the electrically conductive material comprises electrically conductive particles, and a second compartment comprising a second collagen scaffold; contacting the first compartment and the second compartment to form a composite comprising the first compartment and the second compartment; and freeze-drying the composite to form a multicompartment conductive collagen scaffold composite comprising longitudinally aligned pores.

In some embodiments, the scaffold comprising collagen, the first collagen scaffold and/or the second collagen scaffold comprises collagen-glycosaminoglycan (CG). In some embodiments, a collagen concentration of the first collagen scaffold varies as compared to a collagen concentration of the second scaffold. In some embodiments, the collagen concentration for the first collagen scaffold ranges from about 0.5 wt % to about 1.5 wt % and the collagen concentration for the second collagen scaffold ranges from about 1.5 wt % to about 5 wt %.

In some embodiments, the electrically conductive particles are microparticles. In some embodiments, the electrically conductive material comprises electrically conductive polypyrrole. In some embodiments, contacting the scaffold comprising collagen with the electrically conductive material, optionally wherein the electrically conductive material comprises electrically conductive particles, comprises layering or coating the scaffold comprising collagen with the electrically conductive particles. In some embodiments, contacting the first compartment and the second compartment to form a composite comprising the first compartment and the second compartment comprises layering two different suspensions on top of one another, wherein a first suspension of the two different suspensions comprises a first collagen scaffold and an electrically conductive material, optionally wherein the electrically conductive material comprises electrically conductive particles, and a second suspension of the two different suspension comprises a second collagen scaffold. In some embodiments, the method comprises allowing the suspensions to interdiffuse prior to freeze-drying.

In some embodiments, wherein the first and second suspensions are prepared by mixing type I collagen, chondroitin sulfate, and acetic acid. In some embodiments, the freeze drying comprises directional lyophilization. Representative cooling/lyophilization rates are desired elsewhere herein. In representative non-limiting embodiments, the freezing temperature employed, which facilitates control of the compartment production, can range for about −10° C. all the way to the temperature of liquid nitrogen (˜−196° C.).

In some embodiments, the presently disclosed subject matter provides a method of treating a skeletal muscle injury in a subject in need thereof. In some embodiments, the method comprises providing a multicompartment conductive collagen scaffold composite, comprising a scaffold comprising collagen and an electrically conductive material, optionally wherein the electrically conductive material comprises electrically conductive particles, and further comprising longitudinally aligned pores; and implanting the multicompartment conductive collagen scaffold composite at a site of the skeletal muscle injury in the subject. In some embodiments, the multicompartment conductive collagen scaffold composite further comprises a first compartment comprising a first collagen scaffold and an electrically conductive material, optionally wherein the electrically conductive material comprises electrically conductive particles, and a second compartment comprising a second collagen scaffold, and wherein the second compartment is disposed on the first compartment and the pores are longitudinally aligned between the compartments

In some embodiments, the scaffold comprising collagen, the first collagen scaffold and/or the second collagen scaffold comprises collagen-glycosaminoglycan (CG). In some embodiments, a collagen concentration of the first collagen scaffold varies as compared to a collagen concentration of the second scaffold. In some embodiments, the collagen concentration for the first collagen scaffold ranges from about 0.5 wt % to about 1.5 wt % and the collagen concentration for the second collagen scaffold ranges from about 1.5 wt % to about 5 wt %.

In some embodiments, the electrically conductive particles are microparticles. In some embodiments, the electrically conductive material comprise electrically conductive polypyrrole. In some embodiments, the skeletal muscle injury comprises an injury at a muscle-tendon junction (MTJ).

In some embodiments, the multicompartment conductive collagen scaffold composite further comprises longitudinally aligned pores. That is, in some embodiments, the pores in the first compartment and the second compartment are aligned in the longitudinal plane. In some embodiments, the pores are elongated along a longitudinal direction of the compartment. By “elongated” it is meant the pore is long in relation to width. In some embodiments, “elongated” refers to pores with an aspect ratio (length/width) greater than 1.5.

In some embodiments, the second compartment comprising the second scaffold comprises a more mechanically robust composition, such as having high CG content, to mimic a more mechanically robust biological tissue, such as tendon. The first compartment comprising the first scaffold comprises electrically conductive material (e.g., particles) to mimic a tissue such as muscle. In some embodiments, the collagen concentration for the first compartment comprising the first collagen scaffold ranges from about 0.5 wt % to about 1.5 wt % and the collagen concentration for the second compartment comprising the second collagen scaffold ranges from about 1.5 wt % to about 5 wt %.

A gradient zone can be defined across the first and second compartments. The width of this gradient zone can be tuned, such as by adjusting the interdiffusion time prior to freeze-drying as disclosed herein. In some embodiments, a compartment has a diameter/width ranging from about 6-20 mm, and a thickness/height of each compartment ranging from about 4-50 mm. The width, diameter, and/or gradient zone can be controlled and/or affected by modifying interdiffusion time, collagen content, and/or conductive material (e.g., particle) content, as representative, non-limiting examples. In some embodiments, the multicompartment scaffolds can be fabricated with spatially graded conductivity. By way of example and not limitation, since a collagen compartment is non-conductive, the multi-compartment scaffolds have distinct zones with different conductivities. Further, a gradient of conductivity can be present in the interdiffusion zone between the two compartments. In some embodiments, a collagen concentration of the first collagen scaffold varies as compared to a collagen concentration of the second collagen scaffold to define a gradient between the first compartment and the second compartment. In some embodiments, a gradient is defined between the first compartment and the second compartment by varying another parameter when preparing the presently disclosed composite, such as but not limited interdiffusion time, and/or conductive material (e.g., particle) content, such as between the layers, as representative, non-limiting examples. In some embodiments, multi-compartment scaffolds are fabricated by combining an aligned and conductive scaffold with a layering technique that enables creation of graded, continuous interfaces reminiscent of orthopedic interfaces like the MTJ. In one particular example, the compartments comprising scaffolds were created by layering 2.5 wt % non-conductive CG suspension (‘tendon’ compartment) on top of 1.5 wt % conductive CG-PPy suspension (‘muscle’ compartment) prior to freeze-drying. The layered suspensions were allowed to interdiffuse for 30 minutes prior to lyophilization, creating a gradient zone between the two compartments that is fixed during freezing. The result is a scaffold composite with compositionally-distinct compartments joined at a smooth, continuous interface.

In some embodiments, the interdiffusion time ranges from about 15 minutes to 60 minutes, include about 30 or about 45 minutes as particular examples. In some embodiments, pore sizes range from about 50 μm to about 250 μm in diameter, including about 150 μm and about 200 μm as particular examples. In some embodiments, the conductive material (e.g., particle) content ranges from about 0.1 wt % to about 3 wt %.

In some embodiments, cells are seeded to the composite. In some embodiments, seeding the cells comprises adding a cell solution directly to the compartments. Thus, in some embodiments, one or more compartments are deployed either with or without seeded cells. In some embodiments, the seeding cells can comprise muscle-derived cells (including myoblasts and satellite cells), fibroblasts, and neural cells (neural stem cells, motor neurons), and combinations thereof. Other particular examples of seeding cells are disclosed elsewhere herein.

In some embodiments, the first collagen scaffold and/or the second collagen scaffold comprises collagen-glycosaminoglycan (CG). However, any suitable source of collagen as would be apparent to one of ordinary skill in the art upon a review of the instant disclosure can be employed. Additional examples of collagen sources are described elsewhere herein and include but are not limited to type I collagen from bovine tendon and chondroitin sulfate from shark cartilage as collagen and glycosaminoglycan sources respectively. Collagen, including type I collagen, sourced from other species (human, rat, pig) and/or tissues (dermis) can also be used. For the glycosaminoglycan source, in addition to chondroitin sulfate, heparin, heparan sulfate, or hyaluronic acid can be used.

In some embodiments, the electrically conductive material comprises particles, such as but not limited to microparticles. In some embodiments, the electrically conductive particles comprise electrically conductive polypyrrole. Other representative materials include but are not limited to other conductive polymers, such as but not limited to poly(3,4-ethylenedioxythiophene) (PEDOT), polyaniline, and combinations thereof. In addition to particles, the conductive polymer can also be used as a coating of the collagen, such as the collagen-glycosaminoglycan material.

In some embodiments, one or more additional compartments can be added to the first or second compartment, such as on an opposite side of one compartment from another compartment. The one or more additional compartments can comprise a collagen scaffold or can comprise a collagen scaffold and an electrically conductive material. Freeze-drying the composite can occur after adding the additional compartment to the first and second compartment so that for example the three compartments are treated at the same time. This treatment constitutes freeze-drying of the first and second layers, and also the additional layer. Alternatively, the first and second compartments can be freeze-dried and the additional compartment can be added. In some embodiments, the method comprises allowing interdiffusion time prior to freeze-drying.

### IV. EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Overview of Examples 1-2

There is a pressing need for better therapeutic options for complex VML repair. While many tissue engineering strategies focus solely on skeletal muscle regeneration, the presently disclosed subject matter instead takes a coordinated approach for rebuilding nervous, connective, and muscle tissue to restore normal function and movement. In the following exemplary embodiments, the presently disclosed subject matter employs a collagen scaffold with unprecedented spatial control of structural alignment, electrical conductivity, and mechanics to promote functional MTJ repair. This type of approach for skeletal muscle tissue engineering does not currently exist and would be a powerful biological tool for a range of muscle and/or nerve repair applications. The presently disclosed subject matter addresses at least some of the roadblocks in the art through the following representative, non-limiting innovations:

Multi-tissue repair for VML Injuries. Functional muscle regeneration following VML injury involves creation of an integrated, innervated muscle-tendon unit to enable force generation and normal movement. The presently disclosed subject matter addresses this challenge through the application of electrically conductive collagen scaffolds in combination with bioreactor preconditioning of co-cultured muscle-derived cells (MDCs) and neural stem cells (NSCs). Although previous work has demonstrated certain advantages of electrical and/or mechanical stimulation (Goldberg, 1967; Goldspink, 1999; Maleiner et al., 2018) in facilitating cell alignment, migration, and differentiation, combined electromechanical stimulation has not been extensively studied for the 3D construction of skeletal muscle. The presently disclosed dynamic culture system elucidates how electromechanical stimuli affect MDC and NSC phenotype in both in vitro and in vivo myogenesis.

Scaffolds with graded structural, mechanical, and conductive cues. Orthopedic interfaces contain elegant physicochemical gradients that are essential to their proper function and extremely challenging to recreate synthetically. In some embodiments, the presently disclosed subject matter employs a layered freeze-drying approach to fabricate multi-compartment MTJ scaffolds from two different collagen precursor suspensions: a ‘muscle’ suspension containing conductive PPy particles and a non-conductive ‘tendon’ suspension with increased collagen content. The freezing locks in the diffusive gradient at the suspension interface and forms a scaffold with a continuous junction between the compartments.

Connecting scaffold design to early and late markers of functional regeneration. A limitation of current skeletal muscle tissue engineering approaches is the paucity of early stage predictors of long-term functional regeneration. In some embodiments, the presently disclosed subject matter performs multiplexed quantification of plasma cytokine levels to develop cytokine signatures that directly correlate with long-term recovery of muscle function. Using a challenging small animal model of VML/MTJ injury in immunocompetent rats, longitudinal data is collected on the same animals from cytokine levels at early time points and directly correlate these results to force generation, gait biomechanics, and histology outcomes at later time points.

EXAMPLES 1 and 2 evaluate scaffolds mimicking the structural anisotropy and electrical excitability of muscle/nervous tissue to drive functional repair of complex VML defects. Employing instructive tissue engineering scaffolds and bioreactors, EXAMPLE 1 evaluates the combined role of scaffold alignment and electrical conductivity on in vitro myogenesis of MDC and NSC co-cultures, with or without electromechanical bioreactor stimulation. In EXAMPLE 2, small animal VML models and gait biomechanical analysis are employed to evaluate the efficacy of the presently disclosed scaffold and bioreactor-based approach in the repair of a challenging rat VML/MTJ defect model. Together, these EXAMPLES assist in defining a set of scaffold and bioreactor-based signals for achieving complex VML repair and are informative for generating improved musculoskeletal tissue engineering strategies.

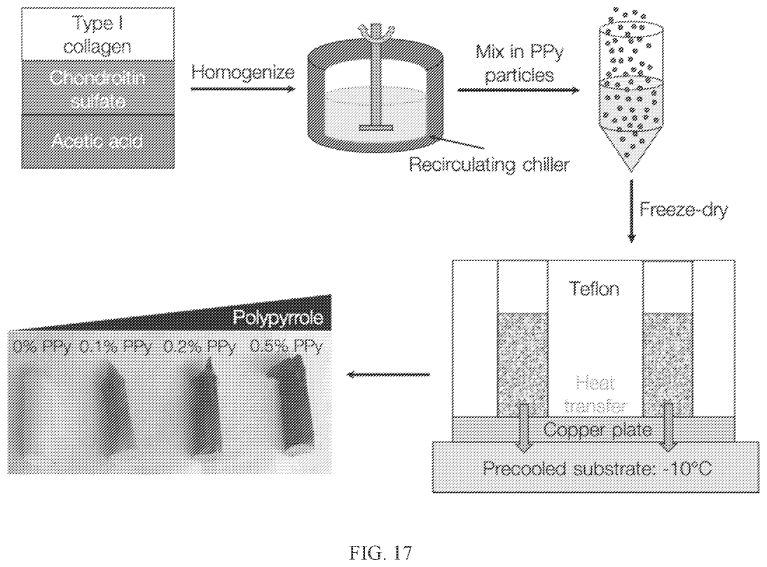

Rationale for selection of material system. EXAMPLES 1 and 2 use a collagen-glycosaminoglycan (CG) scaffold system (Harley & Gibson, 2008). CG scaffolds are chosen in part for their rich history of use in a range of tissue engineering applications including bone (Weisgerber et al., 2016) and tendon (Caliari & Harley, 2011; Caliari et al., 2015b) as well as clinically for peripheral nerve (Godard et al., 2009) and skin (Yannas et al., 1989; Harley & Gibson, 2008) regeneration. CG scaffolds block wound contraction and limit myofibroblast activation, leading to reduced scarring and improved wound healing (Yannas et al., 1989; Harley & Gibson, 2008). CG scaffolds are typically fabricated by freeze-drying where ice crystals template the resultant 3D interconnected macropore structure (Yannas et al., 1989; Pawelec et al., 2014; Alegret et al., 2019). These pores, typically between 50-200 nm in diameter, allow cellular penetration and growth as well as effective biomolecular mass transfer. A directional freeze-drying approach is used to make aligned 3D CG scaffolds for tendon tissue engineering where use of a thermally-mismatched freeze-drying mold as shown in FIG. 17 led to the formation of longitudinally aligned, elongated pores mimicking native tissue organization. For skeletal muscle applications, conductive PPy particles are incorporated directly into the CG suspension prior to lyophilization, highlighting the ability to easily tune precursor suspension composition as an additional benefit of this system. Layering different suspensions prior to freeze-drying can create multi-compartment composites for osteochondral (Harley et al., 2009) and osteotendinous tissue engineering (Caliari et al., 2015b). Therefore, this system is ideal for the facile introduction of conductive polymers into the bulk suspension prior to lyophilization to produce 3D aligned and electrically conductive scaffolds that can also be layered with non-conductive CG content to create multi-compartment MTJ scaffolds.

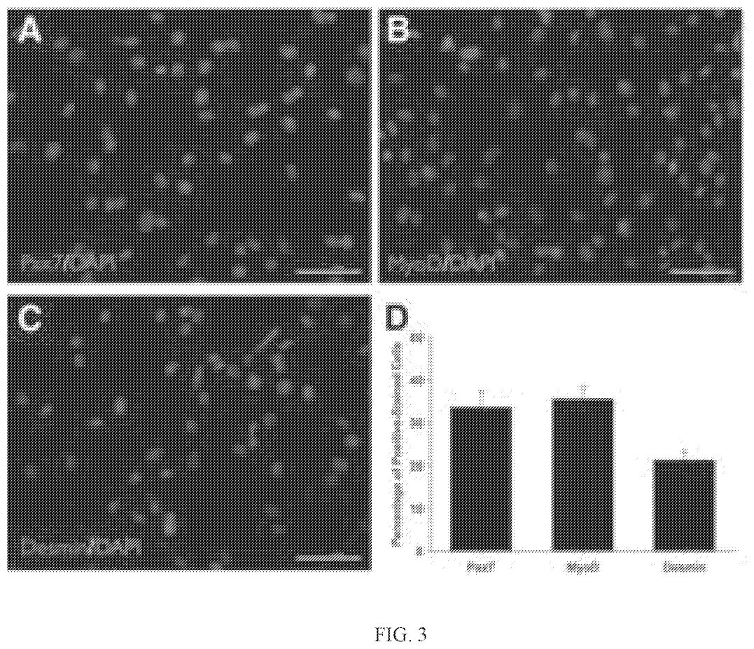

Rationale for selection of cell types. EXAMPLES 1 and 2 use co-cultured rat MDCs and NSCs to study in vitro and in vivo myogenesis. Primary MDCs have been used to repair both tibialis anterior (TA) and latissimus dorsi (LD) VML defects (Machingal et al., 2011; Corona et al., 2012; Corona et al., 2013; Mintz et al., 2019) and represent a mixed population of satellite cells, myoblasts, myotubes, and fibroblasts with 36% of the cells expressing Pax7, 34% expressing MyoD, and 21% expressing desmin as measured by immunocytochemistry (Corona et al., 2012; FIG. 3). Using this mixed population of cells, especially for the in vitro studies described in EXAMPLE 1, is likely more representative of the diverse cell types that can be recruited to the macroporous scaffolds following implantation in EXAMPLE 2. Fetal NSCs, derived from the cortex of E14 rats, are purchased from Gibco and co-cultured with MDCs at low ratios (˜300:1 MDCs:NSCs). These cells are validated as undifferentiated by the presence of Nestin expression and absence of differentiation markers glial fibrillary acidic protein (GFAP), galactosylceramidase (GalC), and doublecortin (Dcx). NSCs are also tested for differentiation potential into neurons, astrocytes, and oligodendrocytes.

Rationale for selection of proteomic targets. In vitro experiments use immunocytochemistry to visualize cytoskeletal organization (F-actin) in addition to myogenic markers MyoD and myosin heavy chain (MHC). MyoD is an important transcription factor in early myogenesis (Tapscott, 2005) while MHC is a later marker than can be used to measure multi-nucleated myotube size and alignment. Neurogenesis is visualized through staining for class III β-tubulin (βIIIT), an early marker of neuronal differentiation (Menezes & Luskin, 1994), and neurofilament 200 (NF200), a neuronal cytoskeletal protein previously used to assay muscle innervation (Mintz et al., 2019). Additionally, NMJ development is visualized by co-localization of βIIIT and MHC with acetylcholine receptor (AChR), the primary mediator of motor neuron-muscle coupling and contraction (Albuquerque et al., 2009). In EXAMPLE 2, tendon healing is evaluated by immunohistochemical staining for scleraxis, a key transcription factor expressed by tenocytes (Schweitzer et al., 2001), and tenomodulin, a glycoprotein highly expressed in tendon (Docheva et al., 2005). Vinculin and paxillin, focal adhesion proteins that are localized to the MTJ (Turner et al., 1991) are also stained for. Muscle vascularization is also evaluated by staining for endothelial cell markers von Willebrand factor (vWF) and CD31.

### Example 1

### Assessing the Combined Ability of 3D Scaffold Alignment and Electrical Conductivity to Drive In Vitro Myogenesis of Muscle-Derived Cell (MDC) and Neural Stem Cell (NSC) Co-Cultures

Representative experimental objectives of this EXAMPLE are to define the scaffold-based structural and conductive cues that combine to promote MDC/NSC viability, myotube maturation, and NMJ development. Scaffold conditions to drive myogenesis without electromechanical stimulation are defined while bioreactor-mediated electrical and/or mechanical stimulation is integrated. Representative milestones include optimization of scaffold properties, including pore size/alignment and conductive polymer loading, to drive myogenesis, determination of desirable seeding ratio of MDCs to NSCs, and tailoring of the bioreactor electromechanical conditioning protocol. Successful milestone completion is evaluated by measuring expression of myogenic and neurogenic phenotypic markers corresponding to myotube and NMJ development.

Representative Data Supporting this Example:

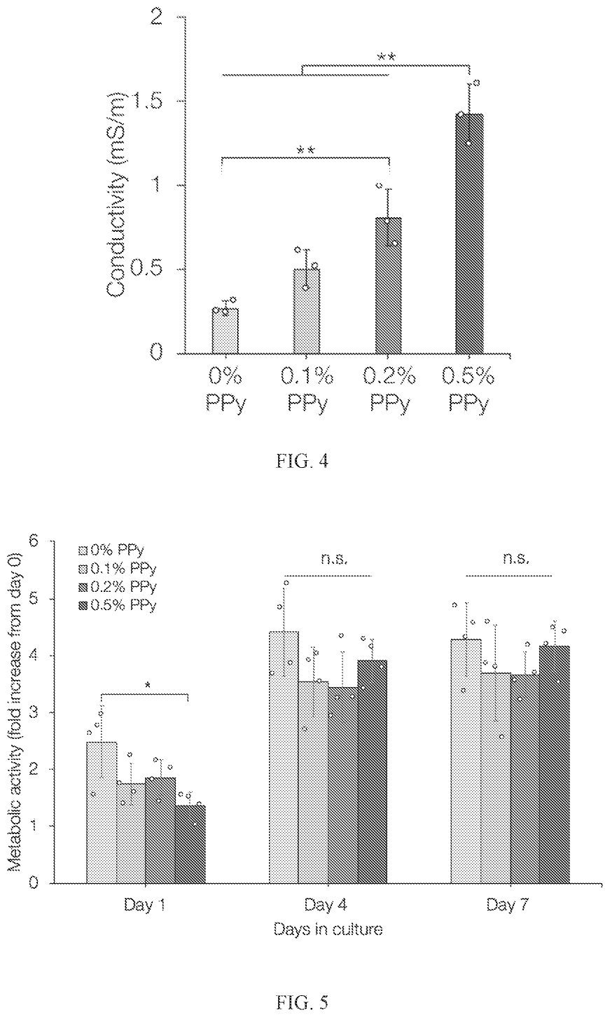

Incorporation of polypyrrole (PPy) microparticles into aligned collagen-GAG (CG) scaffolds improves electrical conductivity. A 3D collagen scaffold with both structural alignment and electrical conductivity for the engineering of anisotropic and electrically excitable tissues like skeletal muscle and peripheral nerve was created. CG-PPy scaffolds are made by simply mixing PPy microparticles with CG content and then freeze-drying in a thermally-mismatched mold to create aligned constructs (see FIG. 17 for fabrication workflow). It was shown that scaffolds incorporating 0.5 wt % PPy content had significantly (˜8-fold) higher conductivity than control CG only scaffolds (FIG. 4). Additionally, it was confirmed that PPy incorporation did not significantly alter the large open pore structure (pore diameter ˜150-200 μm) and pore alignment observed for conventional CG scaffolds without PPy. Scaffold mechanical characterization via rheology showed that hydrated, carbodiimide-crosslinked scaffolds had moduli in a range previously demonstrated to be beneficial for myogenesis (Engler et al., 2006; Gilbert et al., 2010; storage moduli ˜11 kPa) and that PPy incorporation did not significantly affect these values.

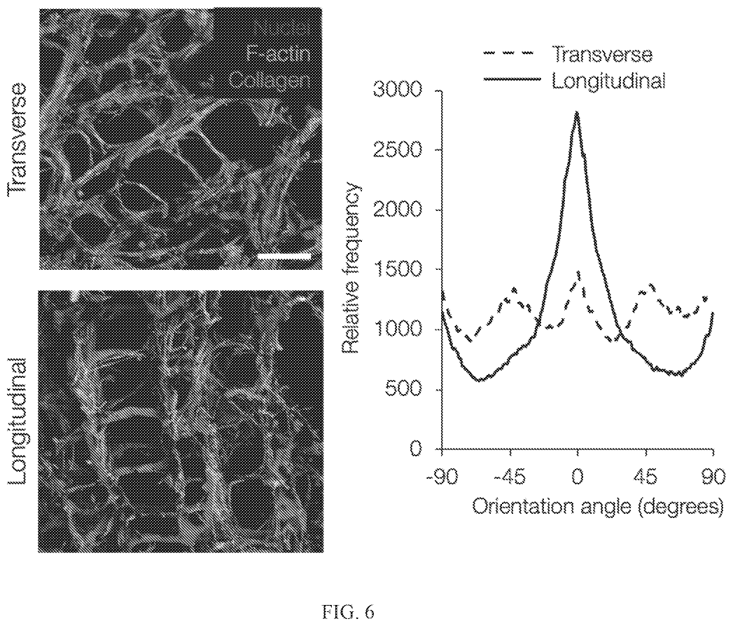

CG-PPy scaffolds support sustained muscle cell metabolic activity and 3D alignment. After characterizing CG-PPy scaffold structural properties, mouse myoblasts were next seeded on the scaffolds to assess their biocompatibility. Cell metabolic activity was tracked over 7 days of culture and showed that scaffolds with PPy content ranging from 0-0.5 wt % all supported sustained, increasing metabolic activity (FIG. 5). Notably, cells spread within CG-PPy scaffolds and conformed to the contact guidance cues presented by the scaffold microstructure, showing robust 3D alignment in the longitudinal plane (FIG. 6).

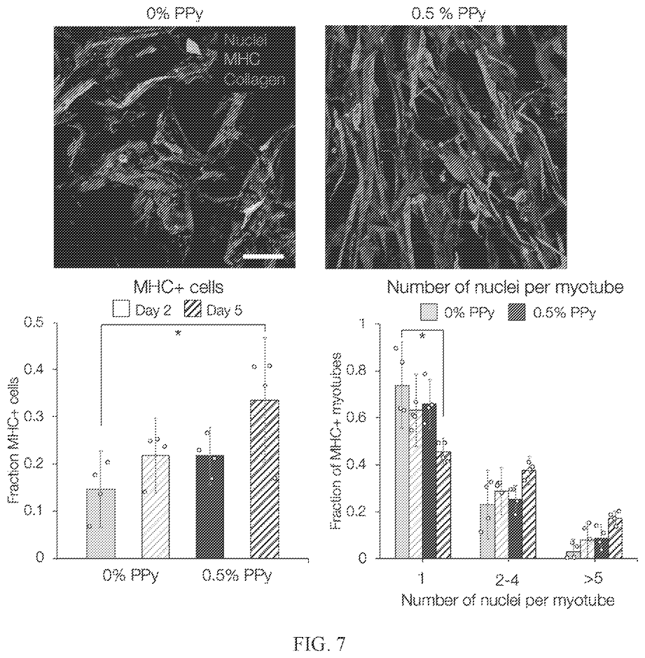

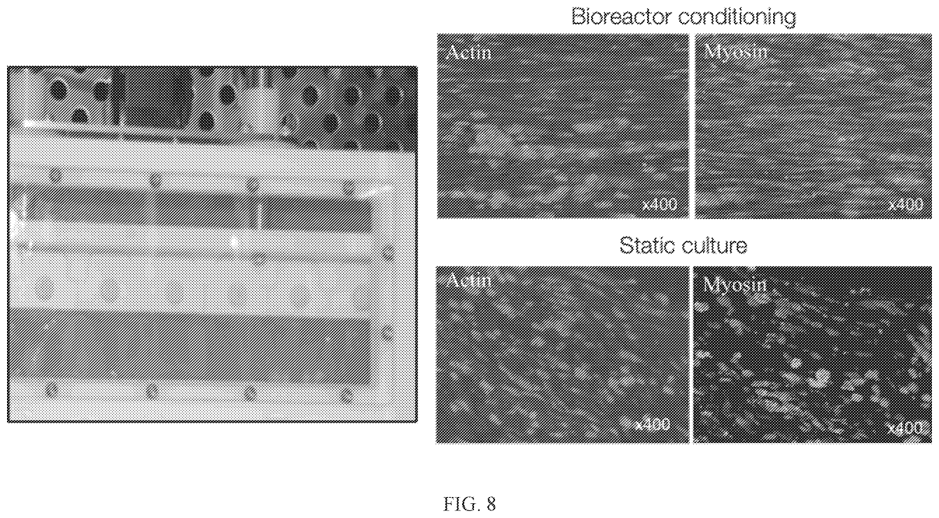

Conductive CG scaffolds support early myogenesis. After showing that aligned and conductive 3D scaffolds that supported myoblast growth could be successfully fabricated, the ability of the scaffolds to guide myotube formation was assessed. A robust image analysis method was established to quantify in vitro myoblast differentiation. Confocal images of seeded cells stained for MHC and counterstained nuclei were acquired, with 40 μm z-stacks used for analysis (skipping 40 μm between stacks to not double count cells). At least a dozen randomly chosen fields of view from at least three different scaffolds per experimental group and time point were analyzed. First, the number of nuclei in MHC-positive cells was counted using a binary feature extractor tool in the BioVoxxel toolbox (ImageJ). The filtered nuclei were then used to quantify the number of nuclei per myotube using the speckle inspector tool in the BioVoxxel. The results were expressed as the percentage of MHC-positive cells and the fraction of MHC-positive cells in myotubes containing 1, 2-4, or greater than 5 nuclei (FIG. 7). Myotube diameter can also be measured using the Diameter) plugin within ImageJ. PPy-containing CG scaffolds had a higher number of MHC-positive cells and about twice as many myotubes with greater than 5 nuclei compared to non-conductive controls, highlighting the potential of CG-PPy scaffolds for skeletal muscle tissue engineering.

Bioreactor conditioning guides cell organization and improves myogenesis. Bioreactor mechanical conditioning of MDC-seeded ECM scaffolds was used to promote improved myogenesis (Moon et al., 2008; Corona et al., 2013) (FIG. 8). The scaffolds were stretched 3 times per minute (10% strain) for the first 5 minutes of every hour using a linear motor-driven custom bioreactor. Bioreactor stimulation promoted MDC alignment and organization in the direction of stretch. Additionally, MDC-seeded constructs subjected to three weeks of in vitro mechanical conditioning were able to generate depolarization-induced contractile force while statically cultured controls did not support force generation.

Experimental Design.

Scaffold fabrication and characterization. Conductive CG scaffolds are fabricated as follows. PPy particles are synthesized via an oxidation reaction in iron (III) chloride for 24 h to form a black precipitate that will be sieved to a diameter <45 μm (Lee & Schmidt, 2015). Alternative PPy fabrication approaches can also be used, including mixing pyrrole monomer with 1H-pyrrol-1-amine monomer at a 10:1 molar ratio (Lee & Schmidt, 2015) to make aminated PPy that can be directly crosslinked to the CG backbone using carbodiimide chemistry (Olde Damink et al., 1996) and using vapor phase polymerization to more evenly distribute PPy content throughout scaffolds (Hanif et al., 2020). CG scaffolds are fabricated by freeze-drying a homogenized suspension of 1.5 wt % bovine microfibrillar collagen and wt % chondroitin sulfate in acetic acid (pH=3.2). For CG-PPy scaffolds, PPy content is mixed into suspension prior to freeze-drying. Aligned scaffolds are fabricated by directional freezing in a thermally-mismatched mold as shown in FIG. 17. Control non-aligned scaffolds are made in aluminum tray molds that facilitate more uniform heat transfer and isotropic pore formation (O'Brien et al., 2005).

Pore size and alignment are quantified from confocal reconstructions of scaffolds with fluorescently-labeled collagen struts and analyzed using a linear intercept macro in ImageJ as previously described (Caliari & Harley, 2011; Caliari et al., 2015b). Pore size is controlled through the freezing temperature where higher temperatures promote ice crystal coarsening and larger pores (O'Brien et al., 2005). Scaffold conductivity is measured using chronopotentiometry in a platinum electrode parallel plate cell (Basurto et al., 2020).

Design of experiments (DOE) approach to evaluate scaffold culture conditions. An aspect of in vitro experiments is to evaluate scaffold culture conditions to simultaneously drive myoblast differentiation, myotube maturation, and NMJ formation. Given the complexity of the experimental design and the desire to probe the combined effects of scaffold structural and conductive cues with MDC/NSC co-culture, a design of experiments (DOE) approach is taken to obtain the maximum amount of information from a reduced slate of experiments. Although DOE approaches have been used for decades, they are still relatively underutilized in tissue engineering (Decaris & Leach, 2011; Rehmann et al., 2016; Levin et al., 2018). JMP software is used to design and employ a fractional factorial design, which typically reduces the number of necessary experimental groups by a factor of 2-4, with primary response variables of MHC expression/myotube size (myogenesis), βIIIT expression (neurogenesis), and MHC/βIIIT/AChR co-localization (NMJ assembly). Experimental factors (variables) include scaffold pore size, anisotropy, and PPy presentation mode along with MDC:NSC co-culture ratio (Table 1).

TABLE 1
DOE factors and levels to test.

| DOE factors (variables) | Levels to test |
|---|---|
| Pore size | 50, 100, 200 μm |
| Pore anisotropy | Non-aligned, aligned |
| Polypyrrole presentation | Unmodified, amine-modified, vapor phase |
| | polymerization |
| MDC:NSC ratio | 1:0, 100:1, 300:1, 500:1 |

Quantification of MDC/NSC behavior in CG scaffolds. The DOE approach guides initial in vitro viability experiments. MDCs from Lewis rats are isolated as previously described from collagenase-digested TA muscles (Kim et al., 2020). MDCs are cultured in low glucose Dulbecco's modified Eagle's medium (DMEM) with 15% fetal bovine serum (FBS) and 1% antibiotic/antimycotic prior to scaffold seeding. Rat fetal NSCs are cultured in StemPro NSC serum-free media (KnockOut DMEM/F12, StemPro NSC supplement, basic fibroblast growth factor, epidermal growth factor). Viability is quantified with Live/Dead staining while metabolic activity will be quantified by measuring alamarBlue (resazurin) reduction to fluorescent resazurin (Caliari & Harley, 2011).

Subsequent experiments focus on identifying scaffold properties and culture conditions that support myoblast differentiation, myotube maturation, and NMJ formation. MDCs and NSCs are seeded onto CG scaffolds and cultured in myogenic differentiation media (F12 DMEM, 2% horse serum, and 1% antibiotic/antimycotic) for 14 days. Myogenic differentiation and myotube formation are assessed using PCR and immunocytochemistry. For PCR, RNA is isolated using RNeasy Mini Kits, reverse transcribed to cDNA, and RT-PCR is performed using SYBR Green chemistry (Caliari et al., 2012) for targets including MHC and βIIIT with GAPDH as a housekeeping gene. For immunostaining, cells are fixed, permeabilized, and non-specific binding sites blocked at room temperature. Cells are then incubated with primary antibodies against MHC, βIIIT, and/or AChR overnight at 4° C. Cells are thoroughly washed in 0.05% Tween in PBS, incubated with corresponding secondary antibodies (including fluorescently-labeled phalloidin to label F-actin), then washed again before counterstaining nuclei with DAPI. Confocal fluorescence microscopy are used to measure cell shape (F-actin), myotube assembly (MEW), neuronal differentiation (WIT), and NMJ formation (MHC/βIIIT/AChR co-localization).

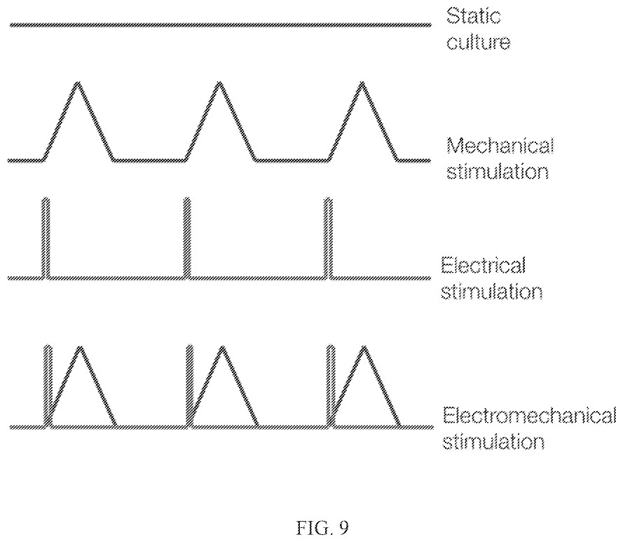

Electromechanical scaffold stimulation to enhance in vitro myogenesis. Mechanical (Moon et al., 2008) and electrical stimulation (Hernandez et al., 2016; Ko et al., 2018) is employed for improving cell alignment, differentiation, and contractility of engineered skeletal muscle following in vivo implantation. However, relatively few studies have explored the synergistic use of electromechanical stimulation to promote skeletal muscle repair (Liao et al., 2008; Maleiner et al., 2018). The application of dynamic bioreactor culture is employed to enhance concomitant myogenesis and neurogenesis using the same evaluation metrics described above. Initial experiments apply bioreactor stimulation to CG-PPy scaffold formulation. Static culture, mechanical stimulation only, electrical stimulation only, and synchronized electromechanical stimulation in aligned CG scaffolds with or without PPy seeded with co-cultured MDCs and NSCs (300:1 ratio) are compared (FIG. 9). Scaffolds are sutured into place and subjected to mechanical stimulation using our previously published protocol (Moon et al., 2008; Mintz et al., 2019): uniaxial mechanical strain are applied at 10% strain for 3 stretches/minute for the first 5 minutes of each hour for 14 days. For electrical stimulation electrodes are inserted at either end of the scaffold. Electrically stimulation proceeds using biphasic rectangular pulses for a duration of 2 ms, 20 Hz, and a voltage of 2.5 V/mm. The electrical stimulation is repeated 3 times/minute for the first 5 minutes of each hour for 14 days to mimic the mechanical stimulation regime (Morgan & Black, 2014). This stimulation strategy was chosen because it has been shown to reduce charge accumulation, improve contractile dynamics, and increase force production in cultured myoblasts (Donnelly et al., 2010; Khodabukus & Baar, 2012; Chen et al., 2019). Finally, these stimulation protocols can be combined to impart electromechanical conditioning where the electrical stimulation immediately precedes mechanical actuation in a manner similar to actual muscle contraction. Following 14 days of dynamic culture, scaffold contractile force generation is evaluated using an external force transducer in a DMT 750 tissue organ bath. The cell-seeded constructs are secured by sutures with one side fixed and the other affixed to the force transducer in growth media. A baseline measurement of passive contraction are collected prior to electrical stimulation. Electrical pulses (30 V, 0.2 ms pulse width at 250 Hz and 30 V, 0.2 ms pulse at 1 Hz) are applied to measure construct tetanic force and twitch contraction in triplicate (Liao et al., 2008; Corona et al., 2012).

Statistical and image analysis. Fixed and stained cell-seeded scaffolds are imaged on a Leica SP8 confocal microscope. 40 lam z-stacks are used for analysis, skipping 40 lam between stacks to not double count cells. A CellProfiler pipeline modified to perform adaptive thresholding are used to quantify cell shape metrics (spreading, circularity) from phalloidin-stained samples. Myotube assembly and maturation are quantified using the ImageJ BioVoxxel and Diameter) tools as described above. NMJ formation is assessed by counting the number of MHC/βIIIT/AChR co-localization spots and NMJ+ myofibers. At least a dozen randomly chosen fields of view (or at least 100 cells for single cell measurements) from at least three different scaffolds per experimental group are analyzed. One-way ANOVA is performed with Tukey's HSD post-hoc tests used to make comparisons between groups. These statistical analyses are conducted using GraphPad Prism 8.0 and R.

Expected results: Primary success metrics are concomitant myoblast differentiation, myotube maturation, and neurogenesis leading to NMJ formation and in vitro force generation. It is expected to establish and validate a toolbox of scaffold structural and conductive instructive cues for driving in vitro myogenesis and neuromuscular integration. We also expect that the DOE approach will allow us to assess agonistic and/or antagonistic interactions between our variables to more clearly understand the complex interplay between scaffold pore size, alignment, conductivity, and MDC/NSC co-culture. Given the showing that CG-PPy scaffolds support equivalent myoblast metabolic activity to non-conductive CG scaffolds, it is predicted that MDC/NSC co-cultures will remain viable for extended culture times. It is expected that that cell organization and alignment will be largely dictated by scaffold structure and not conductivity, especially without bioreactor conditioning, with non-aligned scaffolds fostering reduced myogenesis with smaller, more disorganized myotube formation. It is expected that conductive scaffolds will support increased myotube diameter, length, and number of nuclei per myotube as well as improved NMJ formation. It is further expected that electromechanical stimulation will enhance these effects with mechanical stimulation supporting improved myotube hypertrophy and organization and electrical stimulation specifically improving NSC-MDC coupling through expression of βIIIT and AChR. Finally, it is expected that electromechanical bioreactor stimulation will result in improved tetanic and twitch contraction compared to static culture conditions due to increased myotube maturation and NMJ assembly.

Alternative approaches. While many bioreactor systems have been developed to probe cellular responses to external stimuli, they are often applied to 2D materials or in situ polymerized hydrogels (Liao et al., 2008; Donnelly et al., 2010; Machingal et al., 2011; Martin et al., 2015; Kasper et al., 2018), owing in part to easier substrate integration. Although it is proposed to affix scaffolds in the bioreactor using sutures, alternative methods such as clamps and adhesives can also be employed. The stimulation profiles described have been applied for myogenic cells (Donnelly et al., 2010; Machingal et al., 2011; Corona et al., 2012; Khodabukus & Baar, 2012; Corona et al., 2013; Passipieri et al., 2019) but they have not been validated for MDC/NSC co-cultures. Therefore, both the mechanical and electrical stimulation profiles may need to be systematically evaluated using DOE to improve myogenic differentiation and NMJ formation. If contractile force by electrical stimulation cannot be measured, NMJ development can also be assessed by measuring twitch force before and after administration of the neuromuscular blocking agent d-tubocurarine (Martin et al., 2015), which would ablate NMJ signaling. Finally, while it is proposed using rat cells to be more directly applicable to the immunocompetent rat models in EXAMPLE 2, comparing results with co-cultures of human MDCs and NSCs informs additional clinical applications.

### Example 2

### Determination of the Ability of 3D Multi-Compartment Scaffolds with Co-Cultured MDCs and NSCs to Guide Repair of Musculotendinous Junction (MTJ) Volumetric Muscle Loss (VML) Injuries

This EXAMPLE addresses the combination of scaffold and bioreactor signals that promote innervated functional repair of complex VML injuries. An aligned and conductive CG scaffold formulation is tested in an established TA VML model. This work also informs multi-compartment MTJ-mimicking scaffold design in combination with electromechanical bioreactor stimulation to drive MTJ VML regeneration. Success metrics include restoration of normal force generation and movement patterns.

Representative Data.

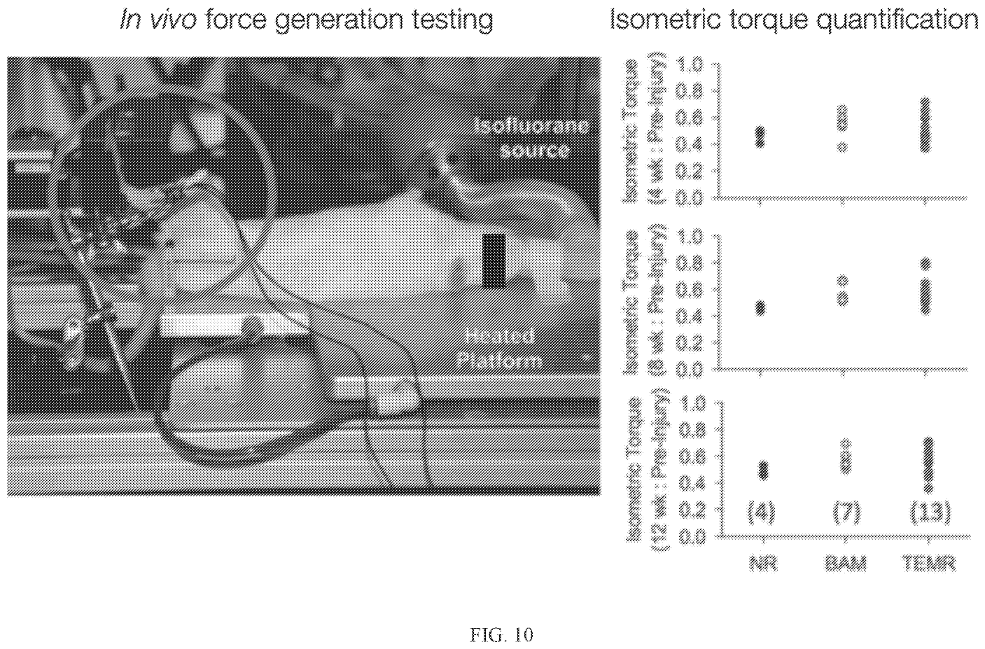

Well-established small animal models of VML. Various VML models (TA, latissimus dorsi) in rodents (mice, rats) can be used to assess functional outcomes after implantation of a variety of tissue engineered constructs (Machingal et al., 2011; Corona et al., 2012; Corona et al., 2013; Mintz et al., 2016; Baker et al., 2017; Passipieri et al., 2017). The TA model is particularly advantageous as it allows repeated in vivo functional testing through quantification of force generation (Corona et al., 2013; Mintz et al., 2016; Mintz et al., 2019) (FIG. 10) and analysis of gait biomechanics (Dienes et al., 2019). Bioreactor conditioned MDC-seeded ECM scaffolds implanted in the TA model lead to increased isometric force generation compared to unseeded controls (Corona et al., 2013). Histological and immunohistochemical staining also highlighted reduced fibrosis (Masson's Trichrome), improved vascularization (CD31/vWF), and neural integration (NF200; Mintz et al., 2019).

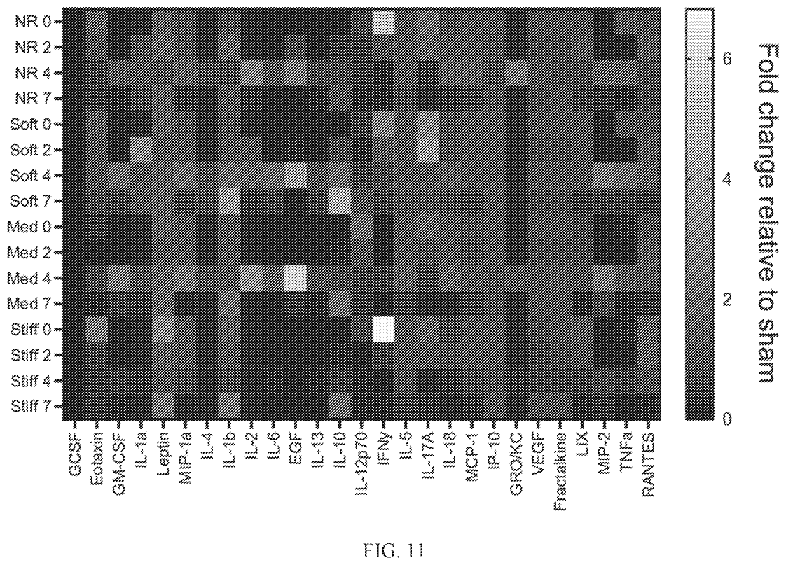

Plasma cytokine quantification to characterize early immune response. Given the role for early, non-invasive markers that are predictive of long-term skeletal muscle regeneration, we previously drew plasma samples from rats within 7-14 days following surgical implantation of hydrogels with variable stiffnesses and performed multiplexed magnetic bead-based quantification of a panel of immunology-related cytokines. Data indicated that hydrogel mechanics influenced both T helper (Th)1/M1 pro-inflammatory and Th2/M2 anti-inflammatory cytokine levels (FIG. 11).

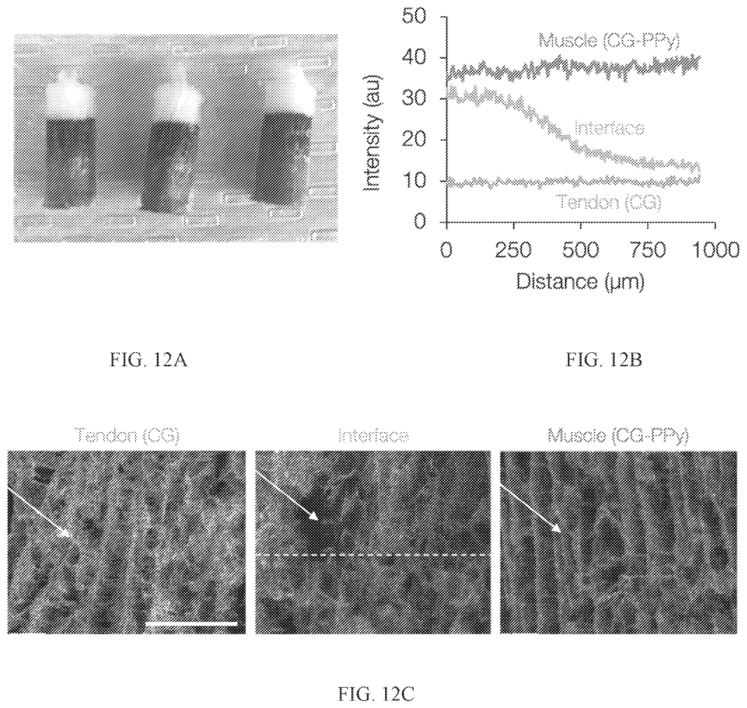

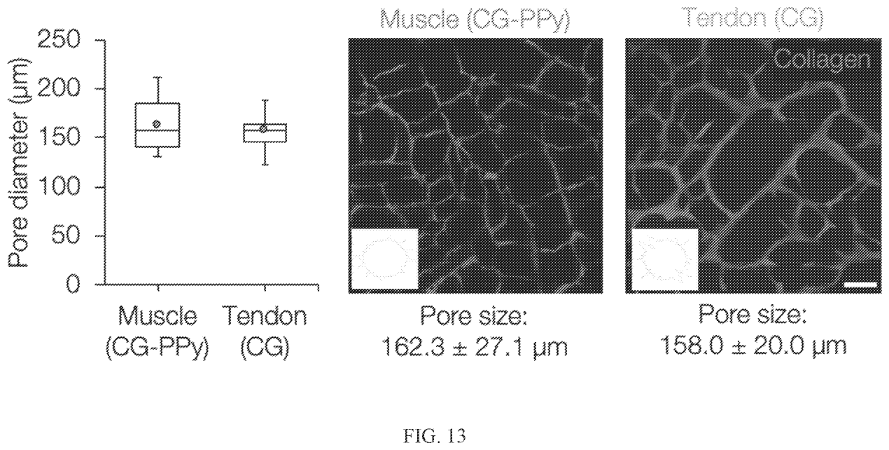

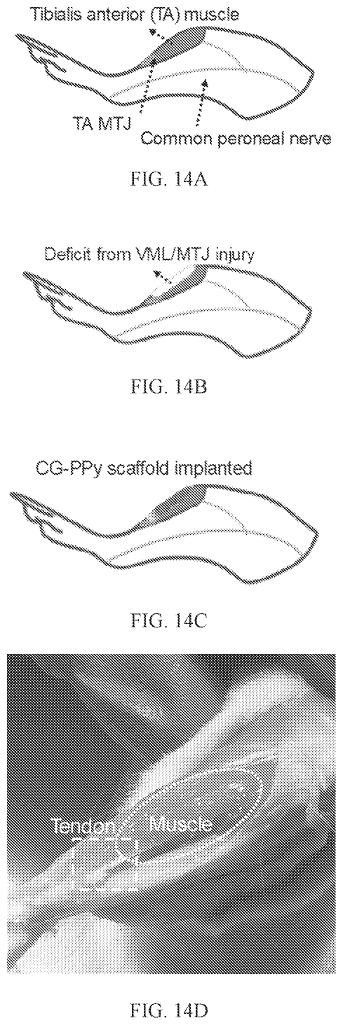

Multi-compartment scaffolds can be fabricated with spatially graded conductivity. Multi-compartment scaffolds are fabricated by combining an aligned and conductive scaffold with a layering technique (Harley et al., 2009; Caliari et al., 2015b) that enables creation of graded, continuous interfaces reminiscent of orthopedic interfaces like the MTJ (FIG. 12). These scaffolds were created by layering 2.5 wt % non-conductive CG suspension (‘tendon’ compartment) on top of 1.5 wt % conductive CG-PPy suspension (‘muscle’ compartment) prior to freeze-drying. The layered suspensions were allowed to interdiffuse for 30 minutes prior to lyophilization, creating a gradient zone between the two compartments that is fixed during freezing. The result is a scaffold with compositionally-distinct compartments joined at a smooth, continuous interface.

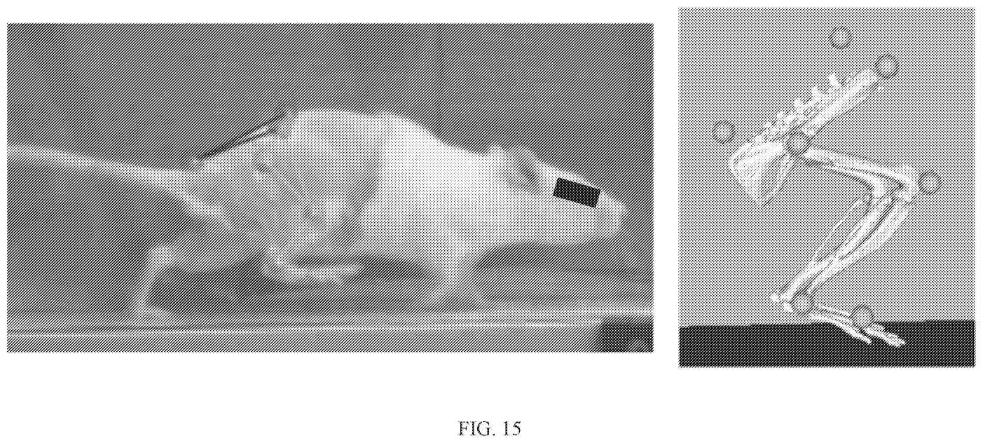

The scaffold interface was characterized by coupling scanning electron microscopy (SEM) with energy-dispersive X-ray spectroscopy (EDS). PPy particles are doped with Cl from their synthesis in iron (III) chloride that can be visualized using EDS elemental mapping. EDS mapping and quantification in FIGS. 12A-12C clearly show higher signal intensity in the CG-PPy ‘muscle’ compartment and a linear signal gradient at the CG/CG-PPy interface. The width of this gradient zone can be tuned by adjusting the interdiffusion time prior to freeze-drying. FIG. 12C also highlights that pores in both compartments are aligned in the longitudinal plane. We additionally characterized pore size from confocal images of fluorescently-labeled scaffolds that were analyzed using an adapted linear intercept MATLAB code (Weisgerber et al., 2016). Results indicated that there was a homogenous pore macrostructure throughout the multi-compartment scaffold with pore diameters on the order of 150 μm (FIG. 13).

Experimental Rationale and Design.

Power analysis and statistics: To minimize the number of animals needed for this study, power analysis and design were focused on the main experimental outcome: recovery of contractile force using in vivo isometric measurements. In this scenario, 8 animals are assigned to each treatment group, which is sufficient based on previous experience to detect a 20% difference between groups with 80% power at alpha=0.05. Repeated functional testing and gait analysis are performed on the same animals 1 week prior to surgery to establish baselines and at intermediate time points (4, 8, 12 weeks) while tissue harvest and histological analysis is performed only at the terminal time point (24 weeks). Two-way ANOVA is performed with Tukey's HSD post-hoc tests used to make comparisons between experimental groups and/or time points.

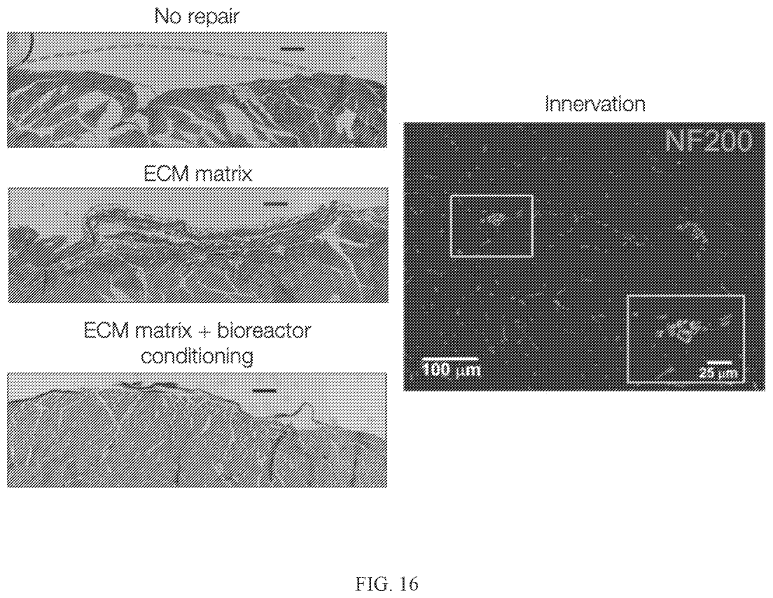

Surgical design. All animal experiments use 250-300 g Lewis rats aged 12-14 weeks at the beginning of the studies. Rats are anesthetized with isoflurane (2.5% to effect and 2% for maintenance), and the surgical site is aseptically prepared. A longitudinal excision is made on the anterior aspect of the lower leg to expose the anteriocrural muscles, including the extensor digitorum longus (EDL) and tibialis anterior (TA). To avoid compensatory hypertrophy of synergistic muscles, EDL and extensor hallucis longus (EHL) muscles are surgically ablated (Corona et al., 2012; Corona et al., 2013). The primary surgical defect is then created by resecting ˜50% of the width of the tibialis anterior tendon from the anterior portion. The muscle injury will then extend up from the resected tendon to include the MTJ and ˜20% of the TA muscle weight (FIGS. 14A-14D). The mass of muscle-tendon tissue to be removed is estimated using a linear regression determined experimentally to estimate TA muscle mass from rat body weight (Corona et al., 2013). The wound bed is compressed with sterile gauze until all major bleeding has subsided. Animals are then randomly assigned to different treatment groups as described below. For scaffold groups, the scaffolds are sutured into place using 6-0 vicryl. Sutures are placed at the site of TA tendon injury, anterior and posterior portions of the TA, and at the proximal area of TA injury. The fascia are sutured back in place using 6-0 vicryl in an interrupted fashion, and the skin is closed with 5-0 monofilament polypropylene in an interrupted fashion. Skin glue may be applied in combination with the sutures as needed. Buprenorphine (0.05 mg/kg, subcutaneous) is administered for 3 days as the primary post-procedural analgesic. Following surgery rat health are monitored daily and skin sutures are removed after 14 days.

A pilot study assesses the influence of scaffold conductivity on TA muscle regeneration. Male or female rats are randomly assigned before surgery to either: i) No repair, ii) CG non-conductive scaffold, or iii) CG-PPy conductive scaffold experimental groups. A second suite of studies evaluates the combined influence of scaffold conductivity and bioreactor pre-conditioning on MTJ regeneration. Male or female rats are randomly assigned before surgery to one of the following scaffold groups: i) No repair, ii) CG, iii) CG-PPy, or iv) CG/CG-PPy layered MTJ scaffold. Scaffold pore size, alignment, and PPy presentation will be informed by EXAMPLE 1 results. Each of the scaffold groups are implanted either i) without cells, ii) with MDCs and NSCs, or iii) with MDCs and NSCs that have undergone bioreactor preconditioning, with MDC:NSC ratio also guided by EXAMPLE 1.

Cytokine profiling. Initial immune response to surgical intervention is measured through 1 mL blood draws taken prior to surgery and at early time points post-surgery (2, 4, 7, and 14 days) to assess levels of T helper (Th)-associated cytokines. Blood plasma is isolated via centrifugation and samples are assayed using a multiplexed Luminex-based approach. A magnetic bead-based kit (Millipore) that assesses 27 immune-related cytokines and chemokines including pro-inflammatory cytokines like interleukins (IL) 1β, 6, 12, interferon gamma (IFN-γ) and tumor necrosis factor alpha (TNF-α) as well as anti-inflammatory/pro-regenerative cytokines such as IL-4, 5, 10, and 13 is employed. These data are used to non-invasively correlate early immune response to later functional outcomes. This approach is motivated by recent evidence highlighting the importance of immune-related cells such as macrophages, in particular the alternatively activated M2 phenotype (Raimondo & Mooney, 2018), in supporting muscle regeneration (Brown et al., 2014; Juhas et al., 2018; Raimondo & Mooney, 2018). Since in some embodiments the presently disclosed scaffolds largely comprise natural ECM components, it is expected that the immune response to steer more toward the Th2/M2 phenotype as opposed to the Th1/M1 classically activated phenotype often observed following implantation of synthetic materials. Furthermore, the ability to directly and non-invasively correlate early markers of regeneration (such as cytokine levels) with later, functional outcomes (force generation, gait biomechanics) has not been achieved for VML repair and is beneficial in the development of new tissue regeneration approaches.

In vivo functional testing. In vivo functional testing is performed at 4, 8, 12, and 24 weeks after surgery using an isometric torque frequency test, in which isometric torque is produced as a function of stimulation frequency (1-300 Hz; Mintz et al., 2016). First, rats are anesthetized and the hindlimb is aseptically prepared as described earlier. The rat is placed in a prone position on a heated platform. The foot is attached to a force transducer with adhesive tape. Two platinum needles electrodes is placed in the posterior compartment of the lower leg along either side of the common peroneal nerve and adjusted until a maximal twitch force is produced. The contractile force of the anterior crural muscles is then assessed through measuring peak isometric tetanic force production as dorsiflexion occurs. Torques at all timepoints is normalized to rat body weight.

Gait analysis. Motion capture is used to evaluate the kinematics and kinetics of the rodent gait as previously described (Dienes et al., 2019) (FIG. 15). Rats are trained to walk on a treadmill during two 20 minute acclimation sessions the week prior to baseline testing. Treadmill speed is set to 40 cm/s (Clarke, 1991). Once the animals have acclimated to walking on the treadmill their hindquarters are shaved under anesthesia and small reflective markers placed on the bony landmarks of the lower limbs, trochanter, knee, ankle, toe, heel, similar to the plug-in gait marker set typically used for human gait analysis. Kinematic data is collected using a Vicon 8-camera motion analysis system in conjunction with a custom calibration frame developed for the treadmill. The location of each marker is digitized at 120 Hz in 3D space. Spatiotemporal gait (stride length, cadence, symmetry) data along with kinetic data (ground reaction forces) of rat gait will be collected using a custom built walkway instrumented with two ATI mini 40 load cells spaced to capture foot strike during walking. Ground reaction forces can also be combined with the kinematic data to calculate individual joint torques. Time points for gait analysis is 1 week before surgery (baseline) and 4, 8, 12, and 24 weeks after surgery.

Histology and immunohistochemistry. TA muscles/MTJs are harvested and fixed in formalin, embedded in paraffin, and cut serially into 7 μm sections. Muscle fiber cross-sectional area (FCSA) and the number of centrally-located myotube nuclei are evaluated via MHC staining. Immunohistochemical staining is also performed to track NSC neuronal differentiation (βIIIT), innervation (NF200, FIG. 16), and NMJ formation (βIIIT/MHC/AChR co-localization). Macrophage infiltration (CD68) and vascular ingrowth into the scaffold (vWF, CD31) are additionally assessed. Masson's Trichrome staining is used to evaluate the extent of fibrosis. Tendon healing is evaluated by immunohistochemical staining for scleraxis (Schweitzer et al., 2001) and tenomodulin (Docheva et al., 2005). Vinculin and paxillin, focal adhesion proteins localized at the MTJ (Turner et al., 1991) are also stained for.

Expected results. Primary success metrics are recovery of muscle force generation capacity and normal gait biomechanics. Previous work showed that the presence of more mature multi-nucleated myotubes was critical for continuous and longer term VML recovery Corona et al., 2012). Therefore, it predicted that that scaffolds seeded with statically cultured cells will display a more immature phenotype and that bioreactor-conditioned scaffolds will result in superior tissue repair at later time points. It is expected that the presence of acellular CG scaffolds will also support increased force production owing to improved passive force generation but that the addition of a cellular component likely will play a role for active tissue contraction (Gilbert-Honick & Grayson, 2019; Passipieri et al., 2019). Additionally, it is expected CG scaffolds to block wound contraction and limit myofibroblast activation, reducing collagen deposition and fibrosis (Yannas & Tzeranis, 2017). In contrast, it is expected that the no repair VML/MTJ defects will result in limited functional recovery with tissue fibrosis and muscular atrophy over the course of the study. It is also expected that early-stage cytokine expression will correlate with functional outcomes, with earlier transition to Th2/M2-like profiles (higher IL-4/IL-10, lower IFN-γ) in the scaffold groups promoting functional recovery. It is also expected that histological and immunohistochemical analyses will corroborate in vivo functional testing. In particular, bioreactor preconditioned multi-compartment MTJ scaffolds will support larger, more organized myofiber development and expression of myogenic (MEW) and neurogenic (βIIIT) markers. Similarly, an increase in the number and maturity (mature pretzel shape vs. naïve oval-shaped plaque morphology) of NMJs in bioreactor preconditioned scaffolds compared to other experimental groups is expected, due to the integration and differentiation of MDCs and NSCs prior to implantation. Moreover, cell-seeded scaffolds will support improved expression of MTJ-specific focal adhesion proteins and tendon-specific markers through crosstalk between tenocytes and myogenic cells (Charvet et al., 2012).

Alternative approaches. A ˜50% injury to the tibialis anterior tendon may prove too severe for subsequent functional testing and result in complete rupture of the tendon. In this case the magnitude of the tendon injury can be reduced or, if necessary, adapted to a TA VML model with a wider range of experimental groups than those described above. Controlling scaffold degradability plays a role in allowing cell migration and maturation while also mechanically stabilizing the wound site. If scaffolds are degrading too slowly (or too quickly), the degradation rate can be modulated by adjusting the extent of crosslinking prior to implantation (Pek et al., 2004). To gain more insight into repair tissue quality, transmission electron microscopy can be used to characterize the organization of collagen fibers in the tendon/MTJ and to analyze de novo sarcomere formation. Cytokine profiles are useful in defining a signature that correlates with functional recovery with the understanding that elevated expression of cytokines traditionally classified as “pro-inflammatory” (or decreased expression of “pro-regenerative” cytokines) is likely oversimplifying the complex immunomodulatory cascade during wound healing. Although several histological targets are proposed, there are many others that can be evaluated to obtain a more complete picture of the repair outcome. For example, there are numerous muscle markers other than MHC that can be investigated, including MyoD, desmin, intracellular calcium channel ryanodine receptor, and junctophilin, a receptor between the plasma membrane and sarcoplasmic reticulum (Corona et al., 2012).

### Examples 3-10

### Aligned and Electrically Conductive 3D Collagen Scaffolds for Skeletal Muscle Tissue Engineering

Introduction to Examples 3-10

Skeletal muscle tissue possesses an innate ability to heal from most injuries by employing an inflammatory response that results in a cycle of degeneration, remodeling, and repair (Carlson & Faulkner, 1983; Bentzinger et al., 2013). However, the magnitude and multi-tissue nature (e.g., muscle, nerve, vasculature) of volumetric muscle loss (VML) injuries compromises the wound healing response and leads to permanent loss of muscle mass and function (Grogan & Hsu, 2011; Corona et al., 2015; Pollot & Corona, 2016). In fact, musculoskeletal trauma constitutes 65% of injuries sustained in recent military conflicts and 92% of soldiers with muscle deformities are classified as VML patients (Owens et al., 2008; Corona et al., 2015). VML-type injuries also account for a significant portion of civilian injuries including myopathies, surgical loss, and traumatic injuries such as car accidents, gunshot wounds, and compound fractures (U. S. B. and J. Initiative, The Burden of Musculoskeletal Diseases in the United States, https://www.boneandjointburden.org/search/node/Muscle).

The inability to more effectively treat VML injuries in both military and civilian populations motivates the development of therapies that can more fully restore muscle form and function. Current approaches include the autologous transfer of healthy skeletal muscle from a donor site to the area of injury followed by extensive physical therapy (Baker et al., 2017). However, this treatment method has proven both expensive and time-intensive, resulting in generally poor levels of functional improvement as well as associated donor site morbidity that limits the size of the injury that can be treated (Corona et al., 2016). Further challenges to effective treatments include the considerable variability that exists between injuries such as size, depth, and location as well as the heterogeneity of affected tissue types (Bursac et al., 2015). As a result, there are no effective treatments currently available to regenerate large areas of musculoskeletal tissue. In an effort to address this important unmet medical need research has focused on tissue engineering/regenerative medicine approaches, specifically the use of acellular extracellular matrix (ECM) scaffolds, seeded with myogenic cell sources for subsequent implantation (Quarta et al., 2017; McClure et al., 2018; Patel et al., 2019). Building on these findings, other approaches have employed rationally engineered biomaterial platforms that provide chemical and physical cues to direct cell survival and more accurately mimic healthy ECM microenvironments (Vandenburgh et al., 2008; Neal et al., 2015; Wan et al., 2015; Nakayama et al., 2019). Numerous studies have combined biomaterials with isolated muscle cells as well as supporting cell types including neurons, endothelial cells, and fibroblasts to augment the repair process (Juhas et al., 2014; Lesman et al., 2011; Baker et al., 2017; Quarta et al., 2017; Perry et al., 2018). While many of these strategies hold promise, they are still limited in their ability to drive functional, innervated, and vascularized muscle repair, based in part on their inability to mimic both the structural organization and the electrochemical excitability of native muscle.

Skeletal muscle development proceeds in a tightly regulated manner as myocytes fuse to form myotubes (Musumeci et al., 2015) and progressively organize into aligned myofibers, fascicles, and larger muscle fiber bundles (Musumeci et al., 2015; Nishimura, 2015). During this process, a complex ECM is deposited surrounding muscle fibers (endomysium), muscle fascicles (perimysium), and the muscle body (epimysium) composed primarily of type I and III collagen (Gillies & Lieber, 2011). The ECM also includes type IV and V collagen as well as proteoglycans such as decorin, biglycan, and laminin (Gillies & Lieber, 2011). It is this anisotropic organization from the molecular to tissue scale that ultimately allows for effective force generation during muscle contraction (Gillies & Lieber, 2011). However, in the case of myopathies and severe trauma like VML, subsequent tissue fibrosis results in disorganization of the ECM and inefficient muscle contraction which ultimately leads to a permanent loss of muscle function (Bursac et al., 2015). The resultant misalignment and disorganization of the ECM after muscle injury motivates the use of regenerative templates that direct anisotropic cell and ECM organization following injury. In addition to the highly organized structure of skeletal muscle ECM, bioelectrical stimuli in the cellular microenvironment can regulate cell fate decisions involved in embryonic development, myoblast differentiation, and tissue regeneration (Weiss et al., 1990; Mihic et al., 2015; Yang et al., 2016a; McLaughlin & Levin, 2018; Alegret et al., 2019; Fennelly & Soker, 2019).

Current methods to simulate endogenous tissue conductivity employ the use of electrically-responsive polymers including polypyrrole (PPy; Ateh et al., 2006), polyaniline (PANT; Qazi et al., 2014), and poly(3,4-ethylenedioxythiophene; PEDOT; Balint et al., 2014). These organic conductive polymers exhibit robust electrical properties while maintaining biocompatibility and chemical tunability (Balint et al., 2014; Guo & Ma, 2018). Additionally, previous work has demonstrated that conductive biopolymers can propagate electrical signals (Burnstine-Townley et al., 2020) to promote cell adhesion and proliferation (Lee et al., 2006; Yang et al., 2016a), organization (Ku et al., 2012), and differentiation (Ku et al., 2012; Sirivisoot et al., 2013) across a range of electrically-excitable cell types even without external electrical stimulation, highlighting the potential of these materials for tissue engineering/regenerative medicine applications. PPy in particular has proven to be a promising conductive material for the repair of electrically-responsive tissues (Schmidt et al., 1997; Browe & Freeman, 2019; Elias et al., 2019; Sun et al., 2019). However, due to difficulties in material processing, such as poor solubility and mechanical brittleness, the majority of conductive biomaterials present cells with a two-dimensional (2D) environment that does not accurately recapitulate the complexity and heterogeneity of their native (3D) environments (Balint et al., 2014; Alegret et al., 2019). Furthermore, conductive polymers alone do not possess the anisotropy and ECM cues (e.g., cell adhesive sites) that are necessary for skeletal muscle repair. This difficulty in processing conductive materials into the 3D aligned structures needed for musculoskeletal tissue engineering has proven to be a limitation in the field. A potential solution is the use of hybrid materials comprising conductive polymers and anisotropic cell-instructive biomaterials to leverage the strengths of both components.

Recent efforts to engineer anisotropic conductive biomaterials have mainly relied upon electrospinning of hybrid material systems composed of biopolymers and conductive polymers (Ku et al., 2012; Subramanian et al., 2012; Chen et al., 2013; Hsiao et al., 2013). In previous work, nanofibrous scaffolds of PAM and polycaprolactone were prepared by co-electrospinning and aligned using a rotating mandrel or an external magnetic field (Ku et al., 2012; Chen et al., 2013). The researchers found that mouse C2C12 myoblast differentiation was improved on both randomly organized conductive nanofibers and aligned fiber bundles (Ku et al., 2012; Chen et al., 2013). Interestingly, the combination of aligned topographical cues and the electroactivity of the nanofibers had a synergistic effect on significantly improving muscle cell differentiation (Ku et al., 2012; Chen et al., 2013). However, electrospun fiber mats are typically dense with small pores that limit the extent of cell infiltration and the formation of large 3D tissue constructs.

For these reasons, macroporous scaffold-based biomaterials offer an attractive platform for skeletal muscle tissue engineering. Porous scaffolds can provide a 3D interconnected pore structure with large enough pores to allow cellular penetration and growth as well as effective mass transfer of nutrients and metabolic waste.

Although previous studies have employed the use of conductive polymers and architectural cues to direct cellular growth and differentiation, a biomaterials approach to simultaneously mimic the 3D, hierarchical organization, and inherent conductivity of skeletal muscle has proven challenging. The development of a biomaterial platform that pairs architectural anisotropy and electrical conductivity in three dimensions could help fill the clinical void for the treatment of VML injuries and other anisotropic and electrically-responsive tissues. Examples 3-10 describe a 3D electrically conductive and aligned collagen-based scaffold as a platform for skeletal muscle tissue engineering and investigated myoblast growth, organization, and maturation.

### Example 3

### Collagen-Glycosaminoglycan-Polypyrrole (CG-PPy) Scaffold Fabrication

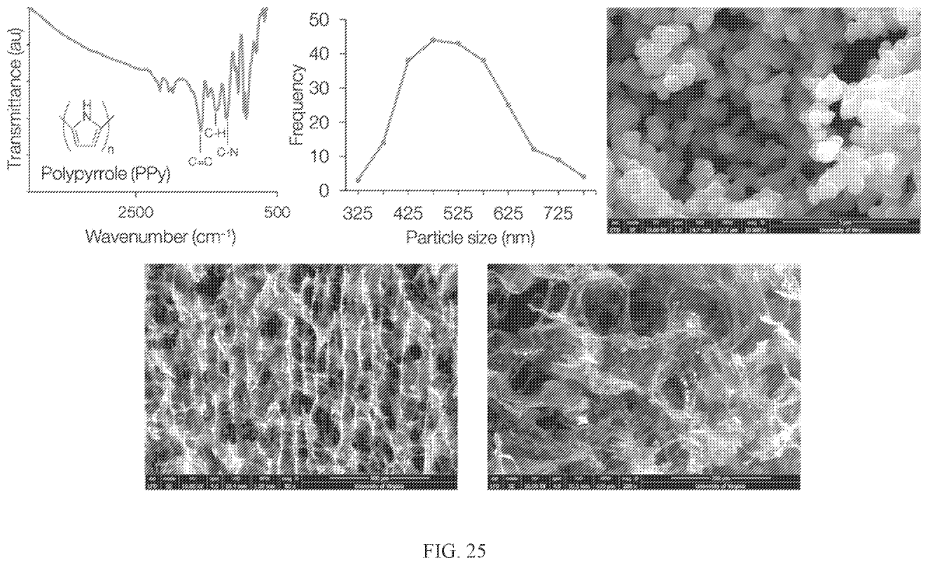

Toward the creation of conductive collagen scaffolds, PPy particles were synthesized via an oxidation reaction with iron (III) chloride (FeCl₃) to yield a fine black powder that could be incorporated into CG suspension prior to scaffold fabrication via freeze-drying. Microscopic and spectroscopic characterization was carried out to determine PPy particle physical and chemical properties. Scanning electron microscopy (SEM) images of PPy particles revealed consistent particle size across samples with an average size of 527.1±96.7 nm (FIG. 25). PPy chemical structure was also analyzed using Fourier-transform infrared (FTIR) spectroscopy. The peak intensity at 1580 cm⁻¹ is associated with the C═C stretching that occurs in the π-conjugated polymer backbone. Additionally, peaks at 1350 and 1220 cm⁻¹ are indicative of C—H wagging vibrations and conjugated C—N in-plane stretching, indicating successful synthesis of PPy (Wang et al., 2005; Fu et al., 2012).

CG scaffolds were fabricated from a suspension of microfibrillar type I collagen and chondroitin sulfate (FIG. 17; Caliari & Harley, 2011). For PPy-containing (CG-PPy) scaffolds the nanoparticles were incorporated into the collagen suspension via vortexing prior to lyophilization. Scaffolds were fabricated via directional freeze-drying using a custom designed mold comprising a thermally conductive copper base beneath an insulating TEFLON® block. The thermal mismatch within the mold facilitated directional heat transfer, causing elongation of the ice crystals in the longitudinal plane during the freezing process, and resulting in anisotropy of the collagen struts following sublimation (Caliari & Harley, 2011). Additionally, control of freeze-drying parameters including initial freezing temperature and rate of cooling allows for user-defined modulation of ice crystal coarsening with higher freezing temperatures and slower cooling rates resulting in larger pores (Divakar et al., 2019). A freezing temperature of −10° C. was used for all scaffolds and has been shown to support cell adhesion, infiltration, and phenotypic stability in various cell types (Gibson & Ashby, 1997; Caliari & Harley, 2011; Caliari et al., 2012).

### Example 4

### Scaffolds Show Longitudinally Aligned Pore Orientation Independent of PPy Content

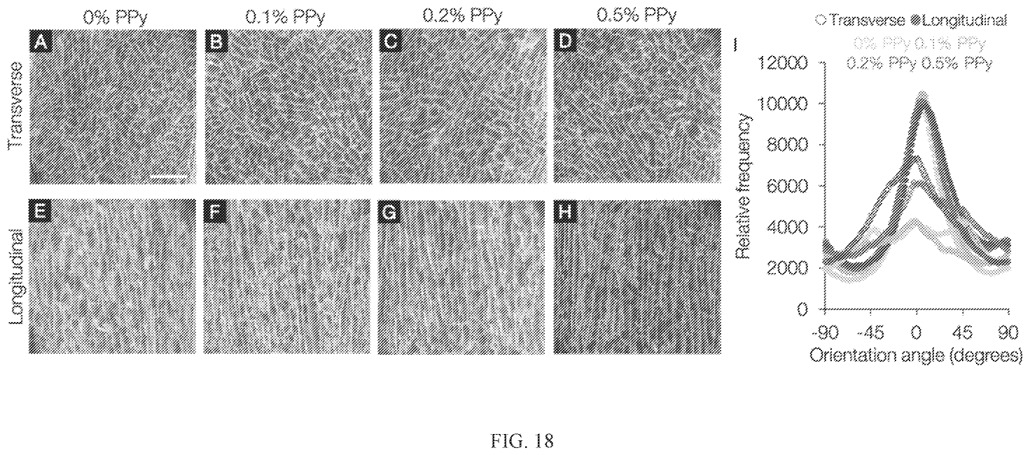

Scaffold pore microstructure for CG materials with a range of PPy content (0, 0.1, 0.2, and 0.5 wt %) was analyzed using SEM. Scaffold anisotropy was then assessed using OrientationJ, an ImageJ plugin. Orientation analysis of scaffold struts showed anisotropic alignment of collagen in the longitudinal scaffold plane corresponding to the primary direction of heat transfer during lyophilization (FIG. 18). Collagen struts showed increased frequency of alignment at 0° (denoting vertical alignment) in the longitudinal plane compared to more randomized fiber organization in the transverse plane. Additionally, orientation analysis showed that the incorporation of PPy particles did not affect the open pore microstructure and resulted in similar pore alignment for all experimental groups.

### Example 5

### Polypyrrole (PPy) Content is Uniformly Distributed Throughout Scaffolds

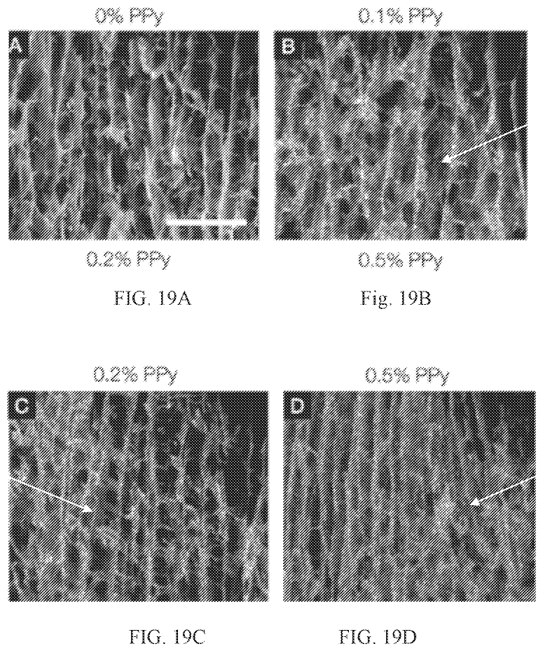

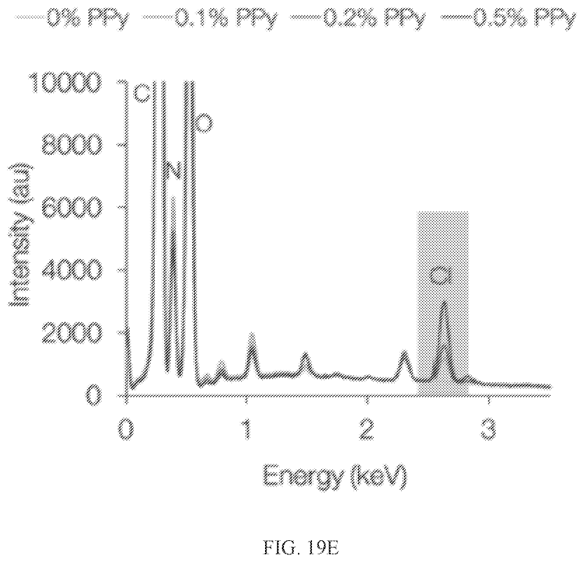

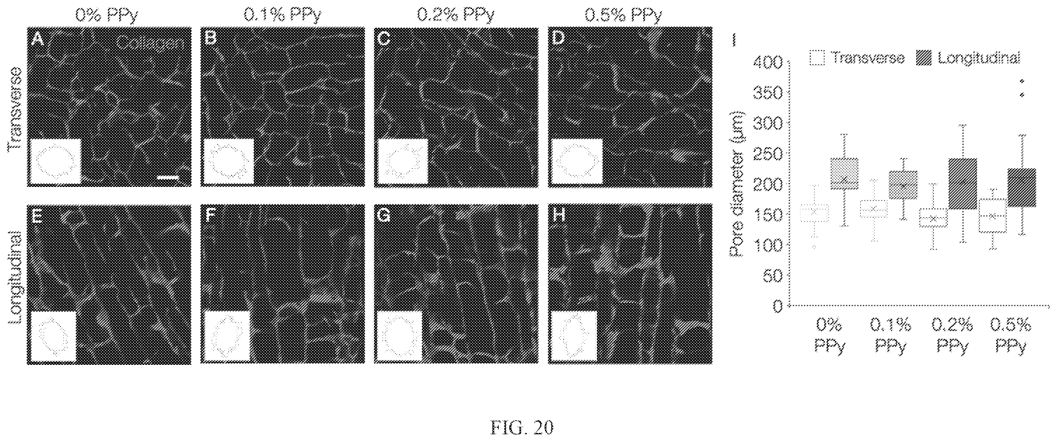

While the addition of PPy did not affect scaffold architecture we also aimed to assess how well distributed the PPy particles were within CG scaffolds using SEM analysis coupled with energy-dispersive x-ray spectroscopy (EDS) elemental mapping. Since PPy particles were synthesized using an oxidation reaction with FeCl₃, the presence of chloride (Cl) dopant within the scaffolds was indicative of PPy localization. EDS mapping showed that PPy was homogenously distributed throughout the CG scaffolds (FIGS. 19A-19E) as designated by the pink pixels (when shown in color) corresponding to Cl content. SEM images also indicated that there was minimal PPy nanoparticle aggregation following scaffold fabrication (FIG. 25). Additionally, the EDS intensity plot shows that the magnitude of the Cl peak increases as the loading of PPy increases and that no Cl was detected in control CG scaffolds.

### Example 6

### Scaffold Pore Architecture is not Affected by PPy Incorporation

After confirming that PPy particles could be incorporated homogeneously in CG scaffolds without affecting pore alignment, we queried if the addition of PPy affected pore size and geometry (Weisgerber et al., 2016). Scaffold pore size (diameter) was analyzed using a custom MATLAB program in which best fit ellipse representations of pores were determined from confocal microscopy images of hydrated scaffolds (Weisgerber et al., 2016). Pores in the scaffold transverse plane generally displayed a rounded morphology with an average pore diameter of 150±26 μm and an average pore cross-sectional area of 17670±532 μm 2 (FIG. 20). In contrast, pores in the longitudinal plane showed an elongated, ellipsoidal morphology with a larger average pore diameter of 203±44 μm and average pore cross-sectional area of 31696±1610 μm². Additionally, the incorporation of PPy particles did not significantly affect the pore microstructure of lyophilized scaffolds in either the transverse or longitudinal planes.

### Example 7

### PPy Incorporation Leads to Significantly Increased Scaffold Conductivity

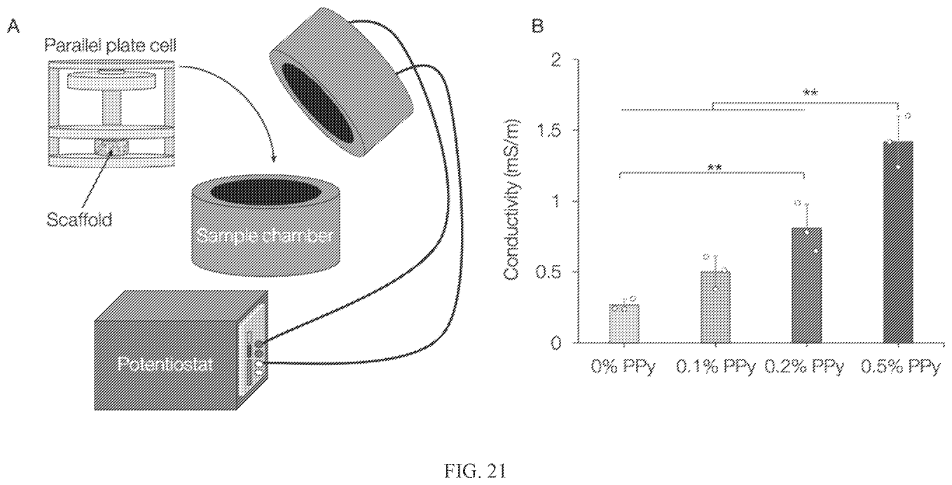

Conductivity of hydrated scaffolds was characterized using chronopotentiometry in a parallel plate cell where electrical potential was measured in response to changes in current. The scaffold conductivity was then related to material resistance using Pouillet's law. The conductivity of the CG scaffolds was modulated by incorporating varying amounts of PPy powder into the collagen-chondroitin sulfate suspension prior to lyophilization. The addition of 0.5 wt. % PPy resulted in an approximately five-fold increase in conductivity (1.42±0.18 mS/m) when compared to the collagen scaffold control without PPy (0.27±0.04 mS/m) (FIG. 21) and was significantly greater than all other experimental groups (P<0.01). Moreover, even lower levels of PPy (0.2 wt %) resulted in a significant increase in conductivity compared to the control scaffold with no PPy.

### Example 8

### CG-PPy Scaffolds Support Sustained and Increasing Myoblast Metabolic Activity

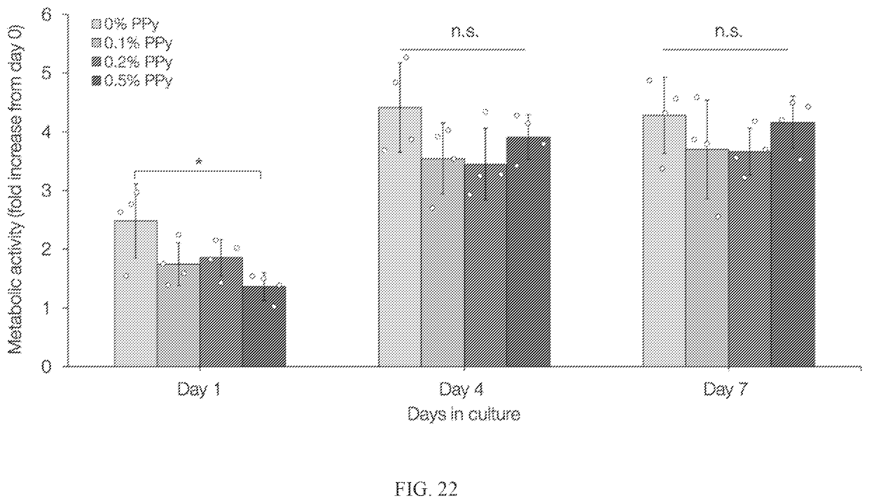

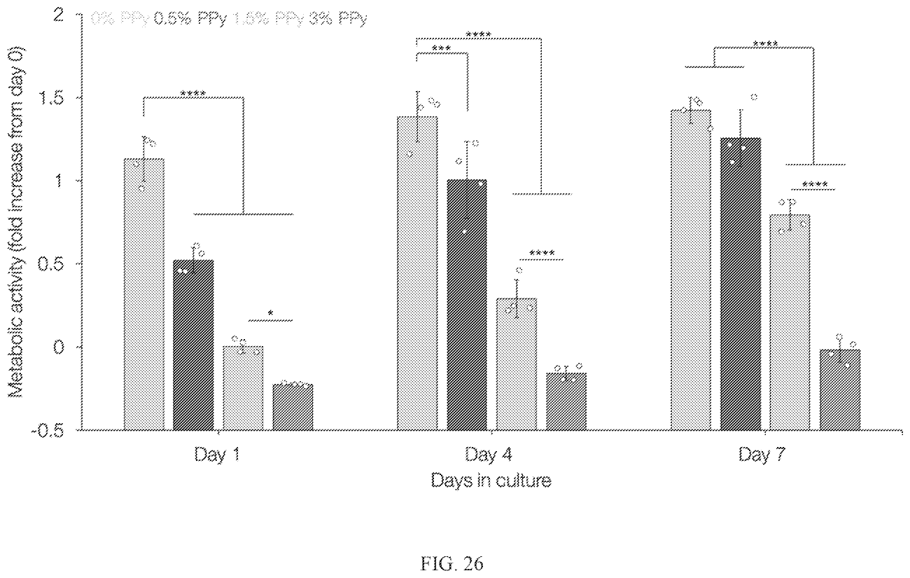

In order to assess how cells would respond to an aligned and conductive 3D environment, mouse myoblast cells (C2C12s) were cultured within scaffolds for 7 days. Cell mitochondrial metabolic activity, as measured by alamarBlue reduction, indicated that CG scaffolds supported sustained and increasing metabolic activity (FIG. 22) across all experimental groups. While initial cell metabolic activity appeared reduced in 0.5 wt % PPy scaffolds after 1 day of culture, metabolic activity was not statistically different between experimental groups after 7 days. These data indicate that the addition of PPy did not detrimentally affect cell mitochondrial metabolic activity. However, preliminary experiments found that increasing the load of PPy in CG scaffolds up to 1.5 and 3 wt % resulted in reduced metabolic activity (FIG. 26), suggesting that PPy can be cytotoxic at high concentrations exceeding 0.5 wt % in the context of these experiments. As a result, due to its superior conductivity and biocompatibility, the 0.5 wt % PPy-containing CG scaffold was compared to the CG-only scaffold control for all subsequent experiments.

### Example 9

### Aligned Scaffolds Guide 3D Myoblast Cytoskeletal Alignment

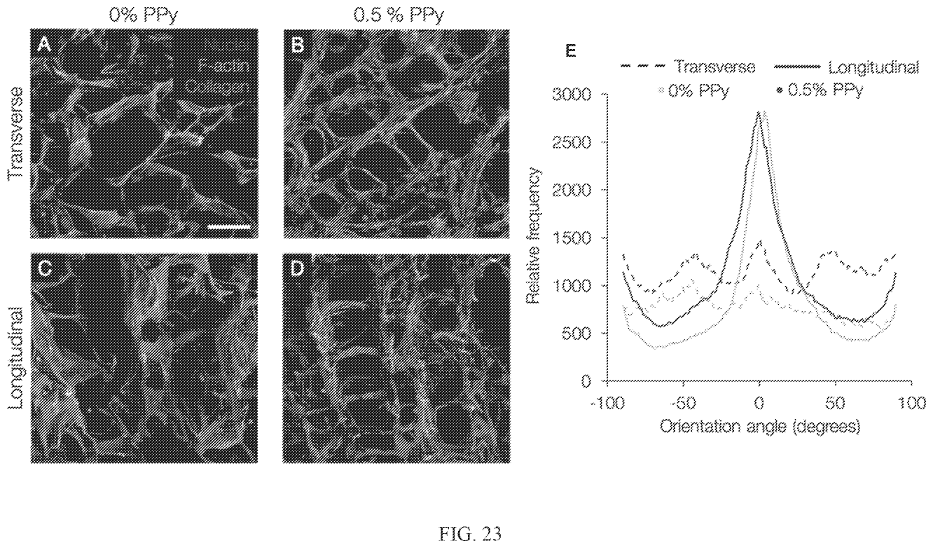

Following the assessment of myoblast metabolic activity, we aimed to determine if 3D pore alignment could provide contact guidance to organize and align myoblasts cultured within the scaffolds. Confocal images of myoblasts were taken after one week in culture from both the longitudinal and transverse scaffold planes and were analyzed to determine the relationship between scaffold microstructural cues and cellular alignment. Confocal images indicated that cells conformed to the scaffold contact guidance cues and organized along the collagen backbone in both transverse and longitudinal planes (FIG. 23). In the transverse plane, where scaffolds contained isotropic rounded pores, cell cytoskeletal orientation angles were randomly distributed. However, in the longitudinal plane, myoblasts showed anisotropic cytoskeletal alignment similar to the high degree of organization observed for the CG scaffold backbone. Additionally, myoblast cytoskeletal organization was similar in both 0 and 0.5 wt % PPy scaffolds, indicating that cell organization was dictated by architectural cues.

### Example 10

### CG-PPy Scaffolds Support Improved Early Myoblast Differentiation

After determining that PPy incorporation did not detrimentally affect cell metabolic activity or inhibit 3D cell alignment within the scaffolds, we aimed to determine if the aligned and conductive scaffold system could facilitate myoblast differentiation. Early C2C12 myogenic differentiation and myotube formation in CG scaffolds were assessed by immunocytochemical analysis of myosin heavy chain II (MHC) and MyoD expression after 2 and 5 days in differentiation media (preceded by 4 days of culture in growth media after initial cell seeding).

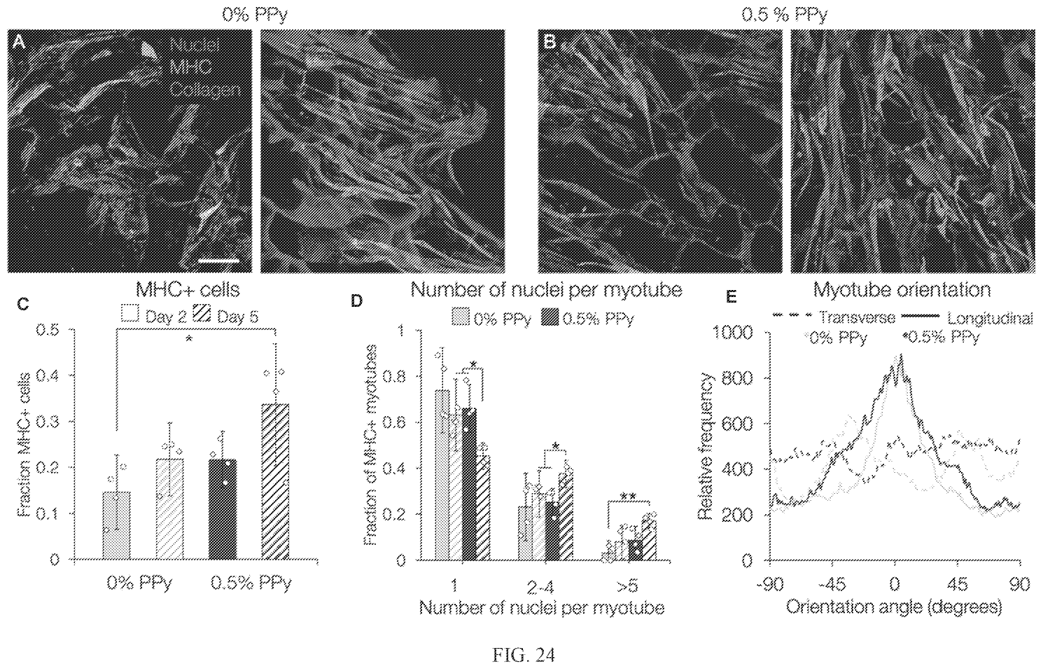

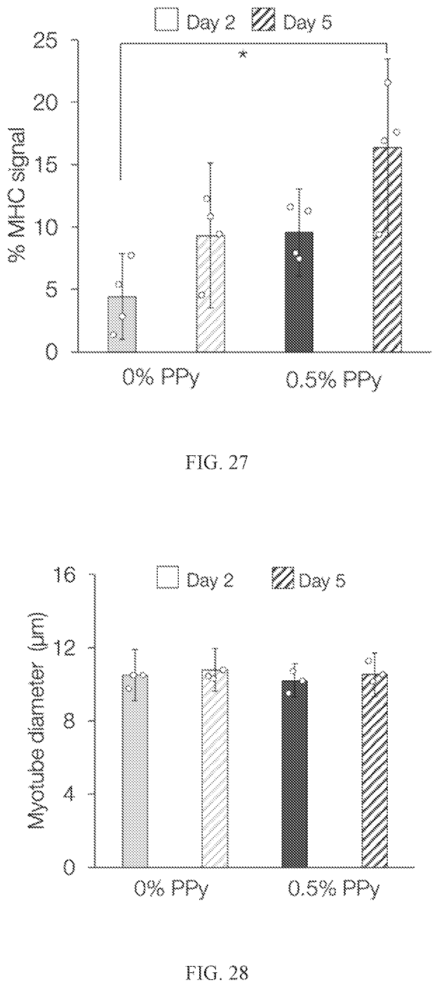

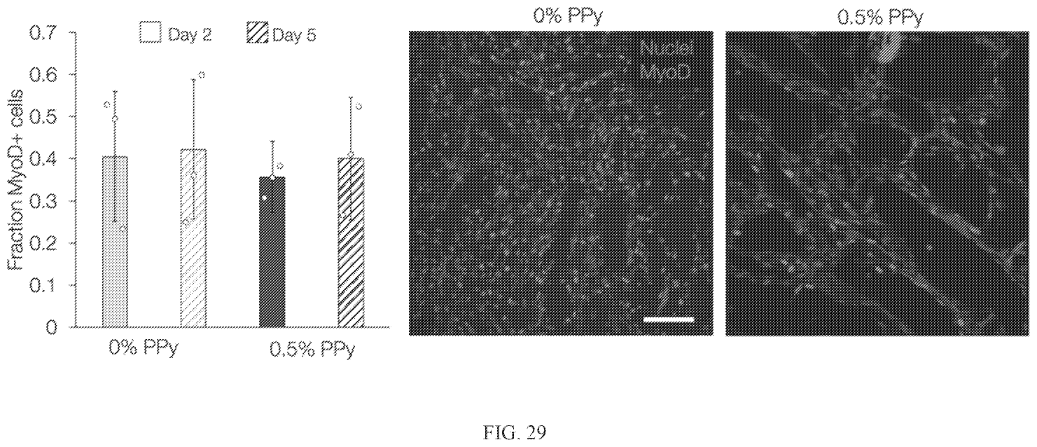

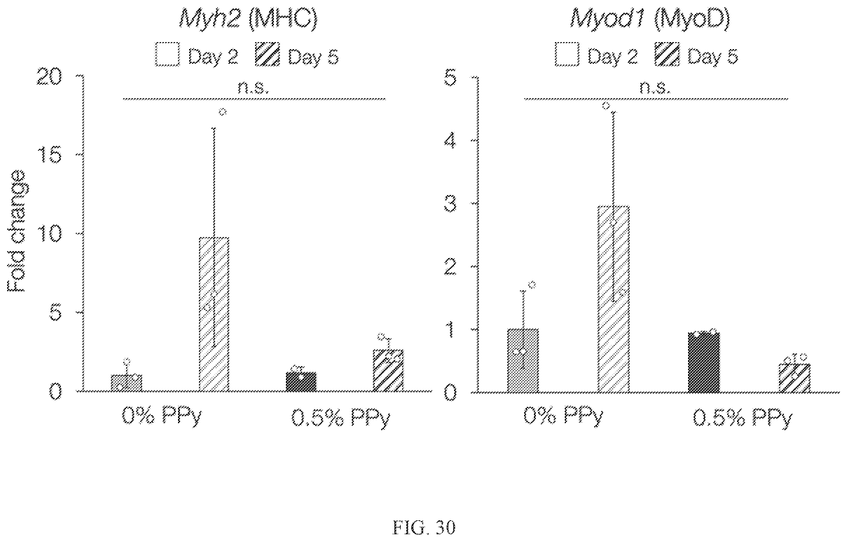

Myoblasts cultured within PPy-containing CG scaffolds generally showed increased MHC staining (FIG. 27) corresponding to a higher fraction of MHC-positive cells (33.6% vs 21.7% at day 5, FIG. 24). CG-PPy scaffolds also supported formation of myotubes with more nuclei; by day 5 of differentiation culture 17.1% of myotubes had 5 or more nuclei while only 7.9% of myotubes in non PPy-containing CG scaffolds reached this level (FIG. 24, Panel D). Moreover, the incorporation of PPy facilitated improved maturation of C2C12s marked by an increased fraction of myotubes containing 2-4 nuclei and a decrease in mononucleated myotubes as compared to non-conductive CG scaffolds. Scaffold microarchitecture also influenced myotube organization with myotubes showing preferential alignment in the longitudinal scaffold plane (FIG. 24, Panel E). Furthermore, myotube organization was similar between 0 and 0.5 wt % CG-PPy scaffolds, indicating myotube alignment was governed by scaffold contact guidance cues. Although we observed increased MHC signal and presence of multinucleated myotubes in PPy-containing scaffolds, there were no significant differences observed in myotube diameter (FIG. 28), MyoD nuclear localization (FIG. 29), or the expression of Myh2 and Myod1 genes encoding for MHC and MyoD (FIG. 30) between experimental groups.

### Discussion of Examples 3 to 10

EXAMPLEs 3 to 10 describe the development of a 3D electrically conductive scaffold with an anisotropic pore structure designed to improve cell growth, alignment, and maturation for skeletal muscle tissue engineering. We chose polypyrrole (PPy) as the conductive component of our scaffold due to its favorable electrical properties, its role in supporting electrically-excitable cell behaviors (Schmidt et al., 1997; Lee et al., 2006; Gilmore et al., 2009; Sun et al., 2019), and its minimal immunogenic response following implantation (Schmidt et al., 1997; Sun et al., 2019). 2D ECM-doped PPy thin films have previously demonstrated the capacity to support C2C12 myoblast adhesion and generation of desmin-positive myotubes (Gilmore et al., 2009). PPy films have also shown the ability to support improved neurite extension of both rat adrenal pheochromocytoma (PC-12) cells and primary chicken sciatic nerve explants when subjected to electrical stimulation as compared to cells cultured on tissue culture plastic (Schmidt et al., 1997). Importantly, these PPy films elicited minimal adverse tissue response when compared to FDA-approved poly(lactic acid-co-glycolic acid) (PLGA) following either subcutaneous or intramuscular implantation 14 weeks post implantation (Schmidt et al., 1997). Furthermore, PPy-coated fibers and films did not elicit any acute systemic toxicity when implanted in vivo (Williams & Doherty, 1994; Zhang et al., 1994).

In this study, PPy particles were chemically synthesized by an oxidizing reaction of pyrrole with iron (III) chloride and added to the collagen-GAG slurry prior to lyophilization. The Cl anion in iron (III) chloride acts as a molecular dopant and renders the PPy conductive. The Cl acts as a charge carrier and when an electrical potential is applied transmits the signal along the π-conjugated polymer backbone (Balint et al., 2014). As a result, the Cl shown in the EDS maps is indicative of the presence of PPy and highlights the uniform particle distribution necessary to allow effective charge transfer throughout the scaffold (FIGS. 19A-19E; Balint et al., 2014).

Following scaffold fabrication, conductivity was assessed using chronopotentiometry. All scaffolds were hydrated in deionized water and crosslinked using carbodiimide chemistry, which on its own has previously been shown to enhance collagen piezoelectric properties (Nair et al., 2019). Scaffolds doped with 0.5 wt % PPy were significantly more conductive than all other experimental groups (1.42±0.18 mS/m) (FIG. 21). However, the conductivity of CG-PPy scaffolds is significantly less than the endogenous conductivity of native/mature skeletal muscle (0.708 S/m; Gabriel (Internet)). This elevated conductivity of muscle tissue is likely due to interactions between other components of the skeletal muscle niche, specifically extracellular fluid, proteoglycans, and the electrically-excitable cells themselves (Hall, 2016). Additionally, there is limited information on the conductivity of the skeletal muscle ECM itself, making comparison with our scaffold material properties difficult.

Moreover, methods to characterize biomaterial conductivity has varied widely in the literature and may account for the large range in reported material electrical properties (Guo & Ma, 2018; Alegret et al., 2019). Previous work using PPy-doped polycaprolactone scaffolds reported conductivity of 10⁻¹ S/cm while other scaffolds prepared from gelatin, bioactive glass, and PEDOT resulted in a conductivity of 210 μS/m (Yazdimamaghani et al., 2015; Zhang et al., 2016). Still other work using PPy-functionalized polylactide and silk reported resistance values on the order of 10³ ohms (Pelto et al., 2013; Severt et al., 2015). Our PPy-doped CG scaffolds fall within this range and we hypothesized that despite the modest increase in CG-PPy scaffold conductivity, it could still prove an effective mediator of the cell-cell interactions necessary to augment myoblast differentiation, especially given the similarity in magnitude to the mV-scale changes in membrane potential associated with myoblast maturation into myotubes (Liu et al., 1998; Fennelly & Soker, 2019). A recent study investigated the combined influence of scaffold alignment and electrical conductivity in a 2D electrospun nanofiber system (Chen et al., 2013). The authors found that the addition of PAM to anisotropically aligned nanofibers resulted in enhanced C2C12 myoblast fusion and maturation compared to non-conductive and isotropic variants, underscoring the synergy of alignment and conductivity on myoblast differentiation (Chen et al., 2013). However, a limitation of the study was that all experiments were confined to 2D constructs that do not mimic the 3D skeletal muscle microenvironment, thus limiting the relevance of the approach to tissue engineering applications.

While previous studies have used freeze-drying to fabricate 3D PEDOT-based conductive constructs for bone (Yazdimamaghani et al., 2015) and neural (Wang et al., 2017) tissue engineering applications, we sought to create 3D conductive scaffolds that also incorporated aligned pores to direct cell and ECM organization in a manner mimicking native skeletal muscle. We successfully applied a directional ice templating and lyophilization approach (Caliari & Harley, 2011; Caliari et al., 2011) to fabricate conductive collagen-GAG-PPy (CG-PPy) scaffolds with highly aligned macropores (FIG. 18). SEM analysis of collagen strut organization showed significant alignment in the longitudinal plane compared to the transverse plane and indicated that the anisotropy was uniform throughout the 15 mm scaffold length. This high level of organization and anisotropy is reminiscent of native/mature skeletal muscle ECM (Gillies & Lieber, 2011).

We chose a freezing temperature of −10° C. for scaffold fabrication to promote slower freezing, increased ice crystal coarsening, and formation of larger (150-200 μm) pores. This large pore structure (orders of magnitude larger than nanometer-scale mesh sizes in most hydrogels) also facilitate for adequate cell infiltration and migration as well as nutrient transport (Harley & Gibson, 2008; Caliari & Harley, 2011; Alegret et al., 2019). In the case of skeletal muscle tissue engineering, a large pore structure is further advantageous because as myoblasts fuse and mature they increase in size and require more space (Musumeci et al., 2015). We have previously shown (Mintz et al., 2019) the median fiber cross-sectional area of rat tibialis anterior muscle is on the order of 1000 μm², meaning that our scaffold (mean pore cross-sectional area of ˜20000-30000 μm²) should initially support the formation of small muscle fiber bundles. Since CG scaffolds are made of naturally-derived components susceptible to enzymatic degradation over the course of weeks to months (depending on the degree of crosslinking; Pek et al., 2004), we predict that gradual cellular remodeling of the scaffold following implantation will permit the formation of larger, more robust muscle fiber bundles. While cytotoxicity of PPy following degradation of the scaffold was not characterized, previous work has shown that PPy-doped materials did not produce significant cytotoxic effects or induce acute system toxicity in a mouse model (Zhang et al., 1994). Additionally, PPy particles injected into the peritoneum of mice did not induce any allergic response, affect spleen, kidney, or liver indexes, or result in chronic inflammation after 6 weeks (Ramanaviciene et al., 2007). As a result, we do not expect that our CG-PPy scaffolds will elicit a cytotoxic response following degradation.

After characterizing scaffold material properties, mouse myoblasts (C2C12s) were seeded and cultured within CG-PPy scaffolds to assess their capacity to support myoblast adhesion and proliferation. CG scaffolds promoted sustained myoblast mitochondrial metabolic activity over 7 days of culture for PPy concentrations up to 0.5 wt %. However, after 1 day in culture 0.5 wt % PPy scaffolds showed reduced metabolic activity compared to other experimental groups (FIG. 22). This reduction is potentially due to lower initial cell attachment from PPy blocking available cell adhesive sites along the collagen scaffold backbone. One potential approach to improve cell attachment would be to modify the PPy with cell adhesive cues; however, additional chemical modifications to the PPy can affect electrical conductivity (Lee et al., 2006). Despite reduced initial metabolic activity, myoblast metabolic activity in the 0.5 wt % CG-PPy group increased roughly 3-fold from day 1 to day 4 with no significant differences in metabolic activity found between any experimental groups at days 4 or 7.

After showing that CG-PPy scaffolds could support sustained myoblast metabolic activity, we assessed the ability of CG-PPy scaffolds to facilitate early myoblast differentiation as measured by myosin heavy chain (MHC) expression and myotube formation. The CG-PPy scaffolds supported an increased fraction of MHC-positive cells and an increase in number of multinucleated myotubes (FIG. 24). However, many MHC-positive cells remained mononucleated, indicative of an immature phenotype. Although our image analysis approach likely underreported the number of nuclei per myotube by excluding some overlapping myotubes within 3D space, it is likely that studies extending culture times in differentiation media past 5 days would result in more robust myoblast fusion and maturation. Similarly, RT-qPCR analysis showed that the addition of PPy did not significantly affect expression of myogenic genes Myh2 and Myod1 encoding for MHC and MyoD respectively (FIG. 30). These results indicate that the passive electrical properties imparted by PPy incorporation were insufficient to increase myogenic gene expression over the course of these experiments. Myogenic differentiation can be assessed over longer culture periods and with the application of an external electrical field.

While immunocytochemical analysis indicated moderately increased myoblast maturation in CG-PPy scaffolds, the impact of cellular interactions with the PPy particles themselves was not characterized in this study. It is possible that interfacial effects such as PPy hydrophobicity and surface roughness, in addition to improved conductivity, could contribute to the changes observed in C2C12 maturation (Fleischer et al., 2014; Ryan et al., 2018; Navaei et al., 2019). However, it has also been reported that electrically conductive scaffolds, even with no external electrical stimulation, can support improved cell adhesion (Shin et al., 2013), proliferation (Qazi et al., 2014; Yang et al., 2016b; Sun et al., 2017), and intercellular communication (Ku et al., 2012; Niu et al., 2018). A proposed mechanism for this behavior is the conductive material's ability to support the creation of electrical fields around electrically-excitable cells (Burnstine-Townley et al., 2020). This can facilitate charge transport on the material-cell interface which can then trigger capacitance coupling between cells and the scaffold (Burnstine-Townley et al., 2020). Primary cell behavior is assessed in CG-PPy scaffolds and the efficacy of CG-PPy scaffolds in in vivo models of volumetric muscle loss injury is appraised.

### Summary of Examples 3 to 10

EXAMPLES 3 to 10 show the development of a 3D, highly aligned, and electrically conductive collagen scaffold via directional lyophilization of a polypyrrole-doped collagen suspension. This composite biomaterial combines aligned CG scaffolds with conductive PPy particles as a platform for skeletal muscle tissue engineering. Directional lyophilization yielded a highly organized pore microstructure mimicking key features of native skeletal muscle that remained intact after the addition of PPy. EDS mapping and chronopotentiometry confirmed that PPy particles were uniformly distributed throughout lyophilized scaffolds (FIGS. 19A-19E), and furthermore, that increased PPy content resulted in higher conductivity. Directional freeze-drying resulted in a macropore (˜150-200 μm) structure with elongated pores in the longitudinal plane that was permissive to cell infiltration and early myotube formation. Analysis of myoblast viability indicated that CG-PPy scaffolds supported increasing metabolic activity and that scaffold anisotropy facilitated cytoskeletal organization along the collagen backbone, similar to healthy skeletal muscle. We also showed that PPy-doped scaffolds supported increases in MHC-positive cells and fraction of multinucleated myotubes compared to CG controls. Together, these results bode well for the application of this material as a scaffold for both in vitro studies of cell behavior and in vivo guidance of skeletal muscle repair.

Experimental Section/Methods

Polypyrrole (PPy) Synthesis

PPy nanoparticles were synthesized via an oxidation reaction with iron (III) chloride (FeCl₃). 4 g of pyrrole monomer was reacted with 200 mL of 36 mmol FeCl₃ using vigorous mixing for 24 hours under ambient conditions (Lee et al., 2006). The resulting black precipitate was isolated via vacuum filtration and repeatedly washed with deionized water until the washings were clear. The powder was then dried overnight in a vacuum oven before being sieved through a 325-mesh (45 μm) screen. Fourier-transform infrared (FTIR) spectroscopy was used to confirm the chemical structure of the PPy particles.

Scaffold Fabrication

Collagen-glycosaminoglycan (CG) scaffolds were fabricated by directional lyophilization from a suspension of microfibrillar type I collagen from bovine Achilles tendon (Sigma-Aldrich) and chondroitin sulfate derived from shark cartilage (Sigma-Aldrich) in 0.05 M acetic acid (Caliari & Harley, 2011). The inclusion of chondroitin sulfate improves the ability to process and homogenize the collagen suspension prior to freeze-drying. Additionally, chondroitin sulfate has been shown to significantly improve CG scaffolds mechanics, including elastic modulus and fracture energy, and support a more open pore structure compared to scaffolds made from collagen alone (Yannas & Burke, 1980). A 1.5 wt % collagen and 0.133 wt % chondroitin sulfate suspension was prepared using a high shear homogenizer (IKA) within a recirculating chiller maintained at 4° C. to prevent collagen denaturation. The suspension was stored at 4° C. until use. PPy-containing (CG-PPy) scaffolds were created by mixing PPy nanoparticles into the collagen suspension via vortexing prior to lyophilization. All scaffolds were fabricated using a VirTis Genesis freeze-dryer (SP Scientific). Following lyophilization scaffolds were dehydrothermally crosslinked at 105° C. for 24 hours.

SEM Analysis

Scanning electron microscopy (SEM) was used to quantify PPy particle size and particle distribution within the CG scaffolds as well as qualitatively assess anisotropy of pore microstructure. SEM analysis was performed with a FEI Quanta 650 scanning electron microscope using a secondary electron detector, backscatter electron detector, and energy-dispersive x-ray spectroscopy (EDS) detector under low vacuum (Caliari et al., 2015b). No coating was applied to scaffolds prior to SEM analysis. PPy particle size was quantified from 5 distinct fields of view for 230 particles using ImageJ's measure function. PPy content was analyzed by EDS tracking of the chlorine content from the iron chloride dopant.

Scaffold Hydration and Crosslinking

Scaffold plugs (7.5 mm diameter, ˜4 mm thickness) were cut from the middle sections of 15 mm length cylindrical scaffolds. Scaffolds were then sterilized in 70% ethanol for 1-2 hours and subsequently washed thrice with PBS. After sterilization scaffolds were crosslinked with using 1-ethyl-3-(−3-dimethylaminopropyl) carbodiimide hydrochloride (EDC) and N-hydroxysulfosuccinimide (NHS) at a molar ratio of 5:2:1 EDC:NHS:COOH where COOH is the carboxylic acid content of the collagen (Olde Damink et al., 1996; Caliari & Harley, 2011). EDC/NHS-mediated crosslinking facilitates the covalent reaction of collagen primary amines with carboxylic acids in both collagen and chondroitin sulfate to improve scaffold mechanical integrity. Previous work has also shown that EDC/NHS crosslinking increases the resistance of CG scaffolds to collagenase and chondroitinase degradation while limiting leaching of the chondroitin sulfate content (Pek et al., 2004). CG scaffolds were incubated in sterile-filtered EDC/NHS solution for 50 min under moderate shaking before being washed twice with PBS. Scaffolds were stored at 4° C. until use.

Pore Size Analysis

Scaffold pore size (diameter) was analyzed using confocal microscopy across the entire length of the freeze-dried scaffold cylinders to account for any longitudinal differences in pore structure. Scaffold sections were cut to ˜4 mm thickness using a razor blade. For imaging, scaffolds were stained with a 2 μM solution of AlexaFluor 568 NHS ester in PBS that conjugates to free amines on the collagen scaffold backbone for 20 min under moderate shaking. After staining, scaffolds were washed twice with PBS before imaging. Both transverse and longitudinal scaffold cross-sections were imaged using a Leica SP5 confocal microscope at an excitation wavelength of 568 nm using a 20× magnification objective. Three 10 μm thick z-stacks (1 μm per slice) were taken of each sample and a maximum projection image was created using ImageJ. Image analysis was performed via a linear intercept method using a custom MATLAB program to compute a best fit ellipse representation of the pore structure (Gibson & Ashby, 1997; Weisgerber et al., 2016). CG scaffolds have previously been modeled using cellular solids theory where the pore structure is defined using a tetrakaidecahedral (14-sided polyhedron) unit cell which has an ellipsoid-like shape (Gibson & Ashby, 1997). The tetrakaidecahedron packs to fill space, nearly satisfies minimum surface energy requirements, and accounts for the structural features of many experimentally characterized low density, open-cell foams including CG scaffolds (Gibson & Ashby, 1997). The pore diameter (d) was calculated based on the values of the major (a) and minor (b) axes of the best fit ellipse using the following equation (Weisgerber et al., 2016):

$$d = \sqrt{\frac{{a}^{2} + {b}^{2}}{2}}$$

Scaffold pore size for both transverse and longitudinal planes was determined from 10 individual images from each scaffold section. 15 scaffolds per experimental group were tested.

Conductivity Measurements

Scaffolds used for conductivity measurements were hydrated and crosslinked using the same methodology described earlier. However, all wash steps and chemical crosslinking were performed in deionized water to avoid the confounding effects of ions in PBS.

The conductivity of the scaffolds was measured using a platinum electrode parallel plate cell. The length (L) of the scaffold was first measured using calipers. Crosslinked scaffolds were blotted to remove excess DI water and loaded into the parallel plate cell. The distance between the electrode plates was then adjusted to ensure uniform contact across the scaffold. EC-LAB® brand software from Biologic Science Instruments was then used to measure scaffold conductivity using chronopotentiometry. The rate of change of electrical potential within the parallel plate electrode was measured by altering current. The resistance (R) of the scaffold was calculated using the slope of the linear regression fit to the chronopotentiometry data using Ohm's law. Conductivity (δ) was finally calculated by Pouillet's law:

$$δ = \frac{L}{RA}$$

where L is length, A is the surface area of samples, and A=πD²/4 where D is the sample diameter (Tayebi et al., 2013). The conductivity of the scaffolds was modulated by incorporating varying amounts of PPy powder into the collagen suspension. Three scaffolds per experimental group were tested.

Cell Culture

Immortalized mouse myoblasts (C2C12s) were acquired from ATCC and used at passages 4 and 5. C2C12s were cultured in standard tissue culture flasks in growth media composed of high glucose Dulbecco's Modified Eagle's medium (DMEM) supplemented with 10 v/v % fetal bovine serum (FBS, Gibco), and 1 v/v % penicillin streptomycin (Invitrogen). Media was changed every 3 days. For differentiation experiments, C2C12s were cultured in media composed of high glucose DMEM supplemented with 2 v/v % horse serum (Gibco) and 1 v/v % penicillin streptomycin. All cultures were performed at 37° C. and 5% CO₂.

Scaffold Culture Conditions

C2C12s were seeded and cultured within CG scaffolds containing 0, 0.1, 0.2, or 0.5 wt % PPy. Hydrated and crosslinked scaffolds were incubated in growth media for 30 min prior to cell seeding to allow for media adsorption. Cultured C2C12s were trypsinized and resuspended at a concentration of 1.25×10⁵ cells per 20 μL media. 10 μL of cell suspension (6.25×10⁴ cells) was added to each scaffold and incubated at 37° C. for 15 min. After incubation, scaffolds were turned over and seeded with an additional 10 μL of cell suspension for a total of 1.25×10⁵ cells per scaffold. Scaffolds were then incubated at 37° C. and 5% CO₂ with growth or differentiation media that was changed every 3 days. For differentiation experiments, C2C12s were cultured in growth media for 4 days before being transferred to differentiation media.

Metabolic Activity

Cell mitochondrial metabolic activity within CG scaffolds was determined using a non-destructive alamarBlue assay. Viable cells within the construct metabolize the active ingredient in alamarBlue (resazurin) into a fluorescent byproduct (resorufin) that can be quantified using a fluorescent spectrophotometer (Caliari & Harley, 2011). Cell-seeded scaffolds were incubated in alamarBlue solution for 1.25 h at 37° C. with gentle shaking. Resorufin fluorescence was assessed via a Synergy 4 BioTek fluorescence spectrophotometer. The relative cell metabolic activity was determined by interpolating scaffold fluorescence readings using a standard curve derived from known cell numbers. Four scaffolds were tested per experimental group.

Immunocytochemistry

All scaffolds were stained with a 2 μM solution of AlexaFluor 568 or AlexaFluor 633 NHS ester in PBS for 20 min under moderate shaking prior to cell culture in order to visualize the collagen scaffold backbone. After the cell culture period, all scaffolds were fixed in 10% formalin for 30 min prior to further staining.

For cell cytoskeletal visualization, scaffolds were cut in half with a razor blade and the cell membranes were permeabilized with 0.1% Triton X-100 in PBS for 30 min. Scaffolds were washed thrice with PBS and incubated with fluorescein phalloidin (1:200 dilution) in PBS for 30 min to stain F-actin.

For primary antibody staining, following fixation scaffolds were washed with 10 mM glycine in PBS for 5 min and cut in half with a razor blade. Cells were then permeabilized with 0.5% Triton-X-100 in PBS for 30 min followed by another three washes for 5 min with mM glycine. Scaffolds were then incubated for 30 min in protein block solution (Abcam) to block non-specific binding. Scaffolds were incubated overnight with antibodies against myosin 4, which recognizes the heavy chain of myosin II, (MF20, mouse monoclonal, eBioscience, 1:200) or MyoD (mouse monoclonal, Santa Cruz, 1:200) in antibody diluent (BD Biosciences). Next, samples were washed thrice with PBS for 5 min and incubated for 2 h with AlexaFluor 488-conjugated goat anti-mouse (1:200) or AlexaFluor 555-conjugated goat anti-mouse (1:200) secondary antibodies. Subsequently, scaffolds were washed thrice with PBS.

Finally, all scaffolds were stained with a nuclear stain, DAPI (1:1000 dilution) in PBS for 5 min before washing once with PBS. Samples were stored in PBS at 4° C. and protected from light until imaging.

Image Acquisition

Fixed and stained scaffolds were imaged using a Leica SP5 confocal microscope equipped with Argon-ion, 405 diode (excitation at 488 nm), and white light laser lines (excitation at 568 or 633 nm) to visualize DAPI, F-actin or secondary antibodies, and collagen channels respectively. A 20×0.7 NA objective was used for image acquisition across all samples. Furthermore, the z-stack capabilities of the microscope were used to image through scaffold sections and produce representative images of cell growth and maturation. A PMT detector was used for visualization of the DAPI fluorophore while hybrid detectors were used to image the collagen backbone, F-actin cytoskeleton, and antibody-based stains.

Assessment of Scaffold Strut and Cell Alignment

Scaffold pore, cell cytoskeletal, and myotube organization were assessed using the OrientationJ plugin for ImageJ. Collagen strut alignment was determined from 30×SEM images using the ‘Distribution’ function within OrientationJ. C2C12 cytoskeletal alignment was measured after 7 days of culture from 25 μm thick maximum projection images of z-stacks collected using a Leica SP5 confocal microscope. Myotube organization was also assessed following 5 days of culture in differentiation media from 40 μm thick maximum projection images of z-stacks collected using the Leica SP5. For all analysis, images were taken of both transverse and longitudinal scaffold orientations. Scaffold strut and cell alignment is reported in terms of orientation angle (−90°-+90°) for samples taken from the transverse and longitudinal planes (Caliari & Harley, 2011). 0° corresponds to the angle of directional solidification in longitudinal sections.

Assessment of Cell Maturation

The number of nuclei in myosin heavy chain (MHC)-positive cells was determined from twelve different fields of view chosen at random across three separate scaffolds per experimental group. First, the number of nuclei in MHC-positive cells was counted using the binary feature extractor tool in the BioVoxxel toolbox. The filtered nuclei were then used to quantify the number nuclei per myotube using the speckle inspector tool in the BioVoxxel toolbox. The results were expressed as the percentages of MHC-positive cells and the fraction of myotubes containing 1, 2-4, or 5 or greater nuclei.

To measure the myotube diameter, the same twelve fields of view from the MHC analysis described above were used. The Diameter) ImageJ plugin was used to quantify myotube diameter from maximum projection images of ˜40 μm thick z-stacks.

In order to evaluate the number of MyoD positive cells, twelve different fields of view were chosen at random across three separate scaffolds per experimental group. The total number of cells was quantified by counting DAPI positive cells using the ‘Analyze Particles’ plugin in ImageJ. An image mask was then made using the DAPI channel and overlaid onto the MyoD channel. Subsequently, the number of MyoD positive cells was determined using the Analyze Particles plugin.

RT-qPCR Analysis

mRNA was extracted from scaffolds after 2 and 5 days of culture in differentiation media using an RNeasy Plant Mini kit. Scaffolds were immersed in lysis buffer supplemented with 10 NM β-mercaptoethanol for 5 min on ice. The lysates were processed following the kit instructions to isolate RNA. RNA was reverse transcribed to cDNA using the QuantiTect Reverse Transcription kit at 1 mg per reaction. Quantitative real-time PCR reactions were performed in triplicate using a Step One Plus Real-Time PCR system (Applied Biosystems) and QuantiTect SYBR Green PCR master mix. All primer sequences were derived from an online primer bank and synthesized by Integrated DNA Technologies. Expression of the following markers was quantified: mouse myosin heavy chain 2 (Myh2), AAGTGACTGTGAAAACAGAAGCA (SEQ ID NO: 1) and GCAGCCATTTGTAAGGGTTGAC (SEQ ID NO: 2); mouse myogenic differentiation factor 1 (Myod1), ATCCGCTACATCGAAGGTCTG (SEQ ID NO: 3) and CTCGACACAGCCGCACTCTTC (SEQ ID NO: 4); and mouse glyceraldehyde 3-phosphate dehydrogenase (Gapdh), AGGTCGGTGTGAACGGATTTG (SEQ ID NO: 5) and TGTAGACCATGTAGTTGAGGTCA (SEQ ID NO: 6). Fold changes in gene expression (normalized to the 0% PPy group at day 2) were calculated using the delta-delta Ct method with Gapdh as the housekeeping gene.

Statistical Analysis

Statistical testing was performed using one-way or two-way ANOVAs with a Gisser-Greenhouse correction for repeated measures followed by Tukey HSD tests using SPSS, R, and GraphPad Prism 9. P values <0.05 were considered statistically significant. Cell metabolic activity data were for n=4 scaffolds per group while scaffold conductivity, pore orientation, and immunocytochemical analyses were for n=3 scaffolds per group. Pore size analyses were for n=15 scaffolds per group. Box plots cover the second and third data quartiles with error bars covering the first and fourth quartiles (or 1.5 times the interquartile range with data points outside this range shown individually). Box plots also include marks for mean (x) and median (bar) data values. Bar graph heights correspond to the mean with standard deviation error bars and individual data points included as scatter plots overlaying the bars.

### Example 11

### Collagen Scaffold Synthesis

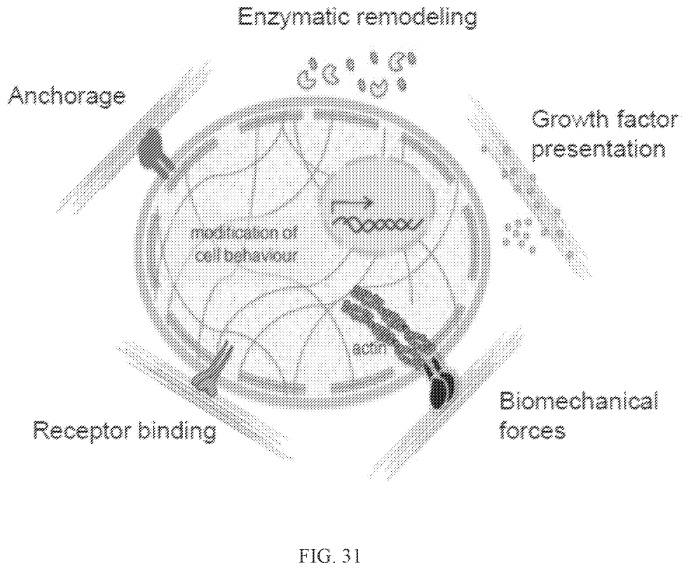

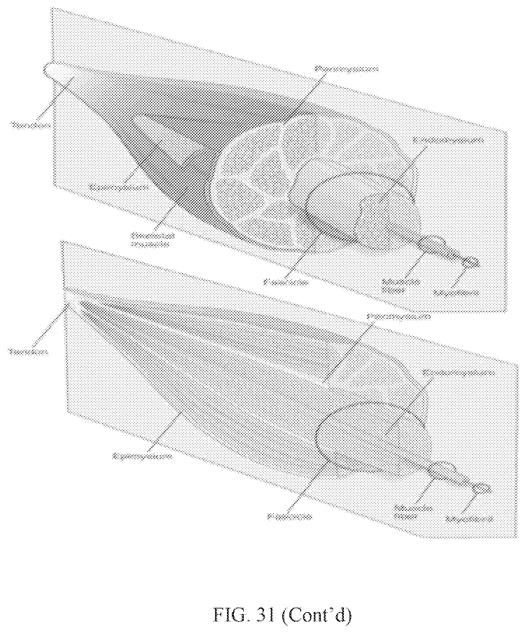

This EXAMPLE describes the synthesis of a multi-compartment scaffold with spatially-defined biophysical cues mimicking the MTJ for repair of complex VML injuries. See FIG. 31. A high solids content CG suspension (‘tendon’ compartment) was layered upon a conductive polypyrrole (PPy)-doped CG slurry to replicate the electrical properties of skeletal muscle and directionally freeze dried. Control of heat transfer during lyophilization enables modulation over scaffold microstructure including porosity and the orientation of collagen struts to mimic skeletal muscle alignment. See FIG. 33, including particularly the lower panel, which refers to freezing, reducing pressure, and solvent sublimation.

In this Example, compartments in accordance with the presently disclosed subject matter were are also tested separately and individually. The results from the single compartment scaffolds discussed herein below support o use of the multicompartment scaffolds upon implantation in rat volumetric muscle loss defects.

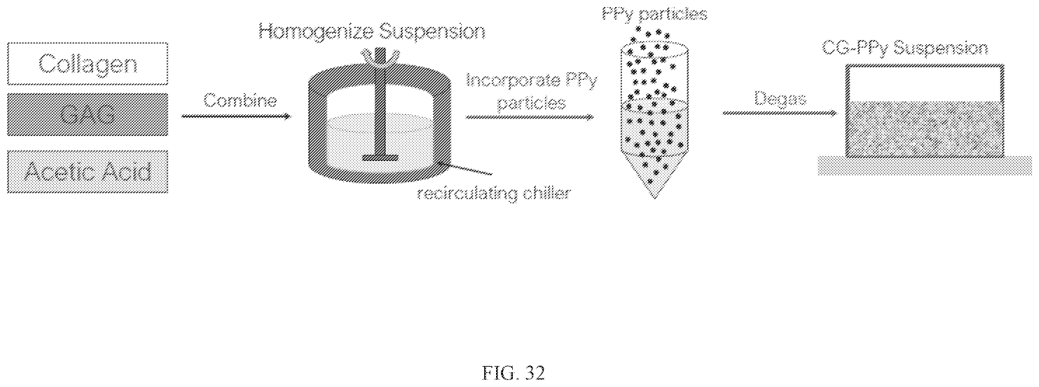

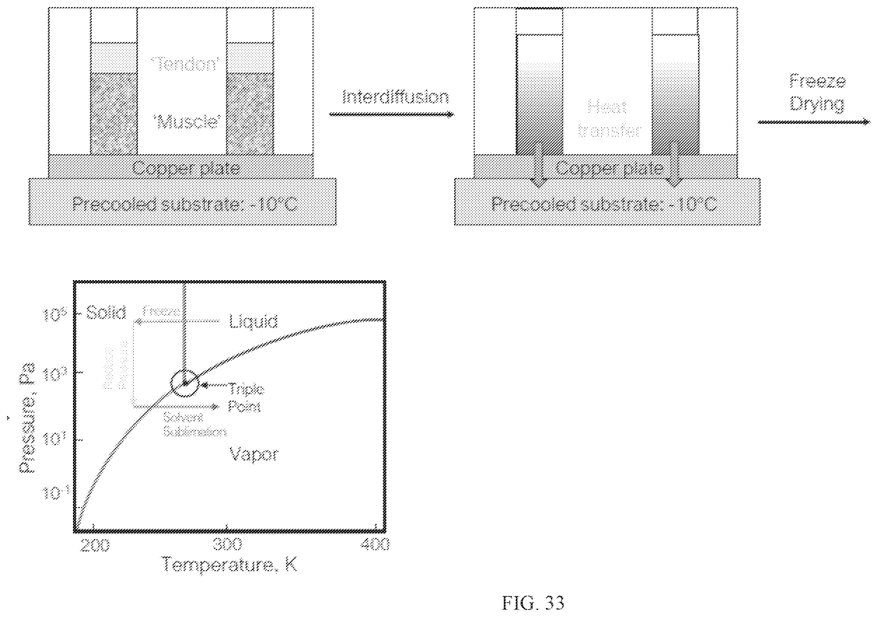

Using an approach shown schematically in FIGS. 17 and 32, a suspension of type I collagen, chondroitin sulfate, and acetic acid was prepared using a high shear homogenizer in a recirculating chiller to prevent collagen denaturization. Conductive PPy particles were synthesized via an oxidation reaction with FeCl₃ and vortexed into the suspension. The suspension was degassed to remove bubbles from the slurry. Referring to FIG. 33, a 2.5 wt % CG suspension (‘tendon’ compartment) was added atop a 0.5 wt % PPy-doped CG suspension (‘muscle’ compartment) and allowed to interfuse for 30 min. The slurry was then directionally freeze dried using a thermally-mismatched mold to recreate the graded interface of the MTJ.

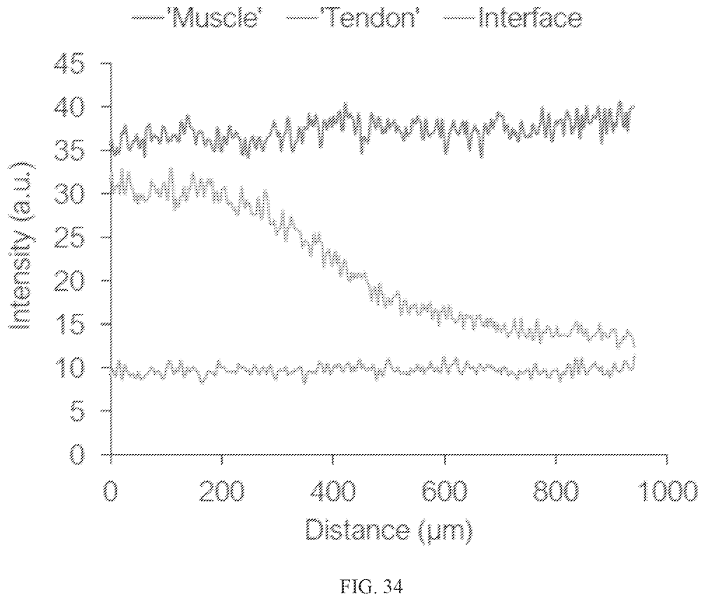

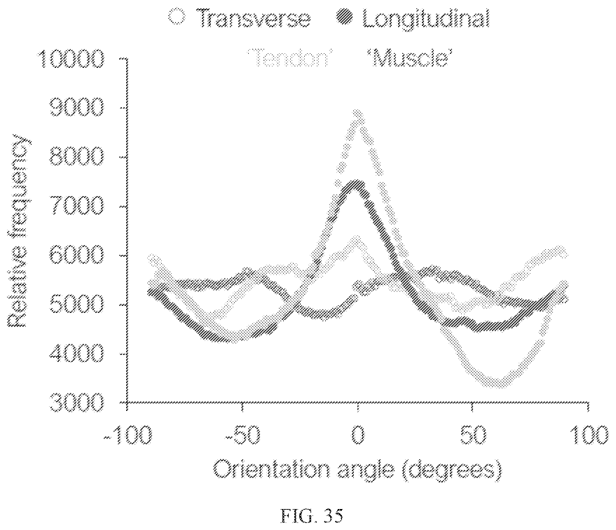

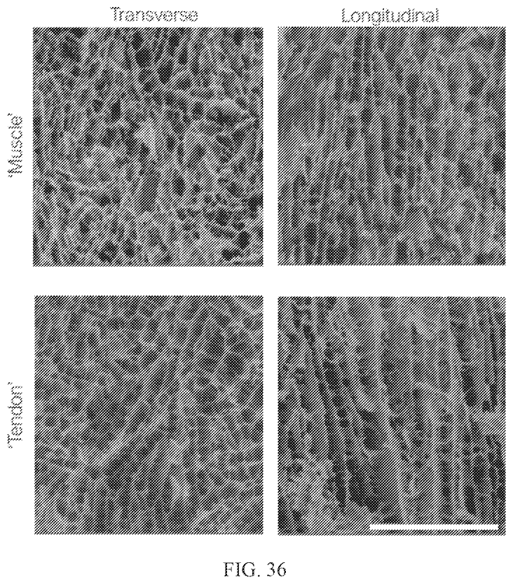

Referring to FIGS. 12A-12C, 13, 34, 35, and 36, representative images of lyophilized MTJ scaffolds with stratified biophysical properties are shown. Energy dispersive spectroscopy (EDS) measurements of chlorine (Cl) content, indicative of PPy, showed high levels of PPy in ‘muscle’ compartments, low levels in ‘tendon’ sections, and a diffusive gradient at the interface. Dashed line indicates interface between ‘muscle’ and ‘tendon’ compartments. Scale bar: 500 μm. Orientation analysis shows isotropic pore structure along the transverse plane and highly aligned, elongated pores, along the longitudinal plane mimicking native skeletal muscle and tendon tissue architecture. Scale bar: 1 mm. Open boxes/circles: transverse plane, Closed boxes/circle: longitudinal plane. Pore microstructure was assessed using ImageJ and MATLAB. Directional heat transfer during lyophilization resulted in scaffolds containing a homogeneous 3D pore microstructure with an average pore size of 162.3±27.1 μm (‘muscle’ compartment) and 158.0±20.0 μm (‘tendon’ compartment) in the transverse plane. n=3 scaffolds.

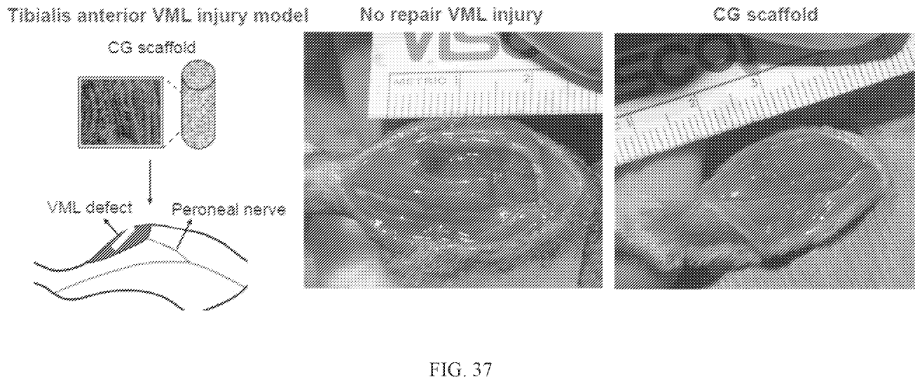

Referring to FIG. 37, a schematic of the VML injury model to the middle third of the left TA is shown. VML injuries were reproducibly created by surgically resecting ˜20% of the TA muscle weight. CG scaffolds were able to fill the VML defect, conform to the injury dimensions, and remain in place following suturing. Scaffolds with graded biophysical properties, reminiscent of the MTJ, can be fabricated via a multi-compartment layering technique and directional lyophilization. Multi-compartment scaffolds contain homogenous pore alignment and architecture. Both conductive and non-conductive CG scaffolds support improved functional muscle recovery in an in vivo model of VML.

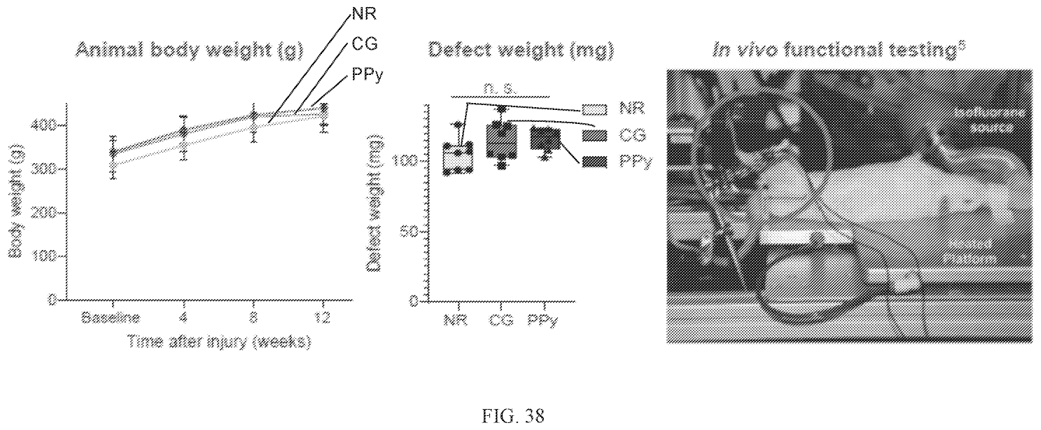

Referring to FIG. 38, an animal body weight showed a healthy weight gain in all treatment groups over the course of 12 weeks that was not statistically different between groups at any time point (two-way ANOVA, P >0.05). Creation of the VML defect was reproducible across all experimental groups with no significant differences observed (one-way ANOVA, P >0.05). Image of in vivo functional testing of the TA following muscle VML injury in which the left leg of an anesthetized rat was attached to a force plate and repeatedly stimulated using electrodes placed along the peroneal nerve.

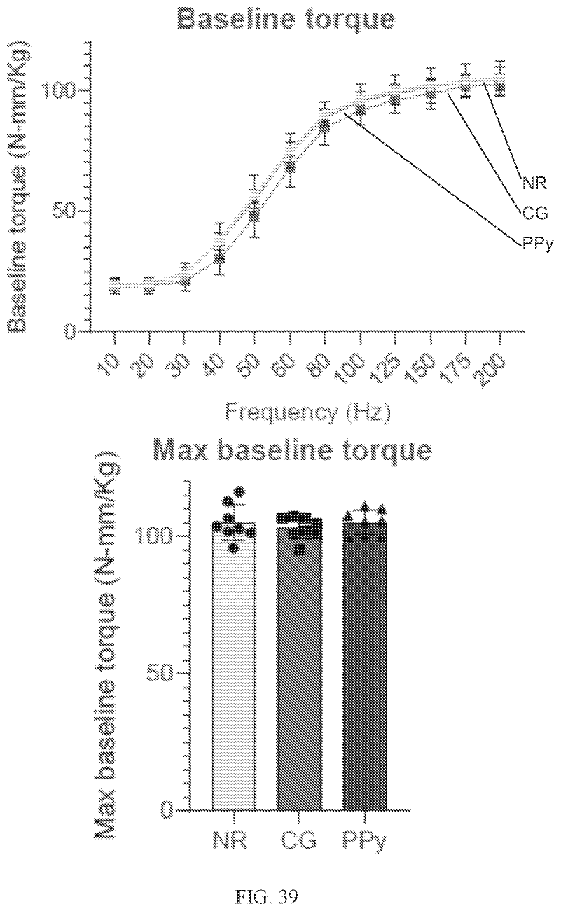

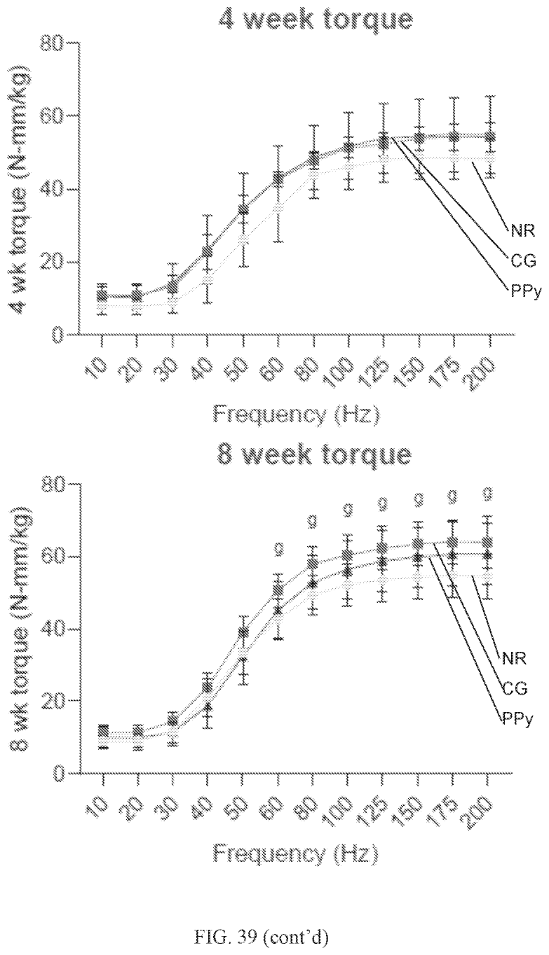

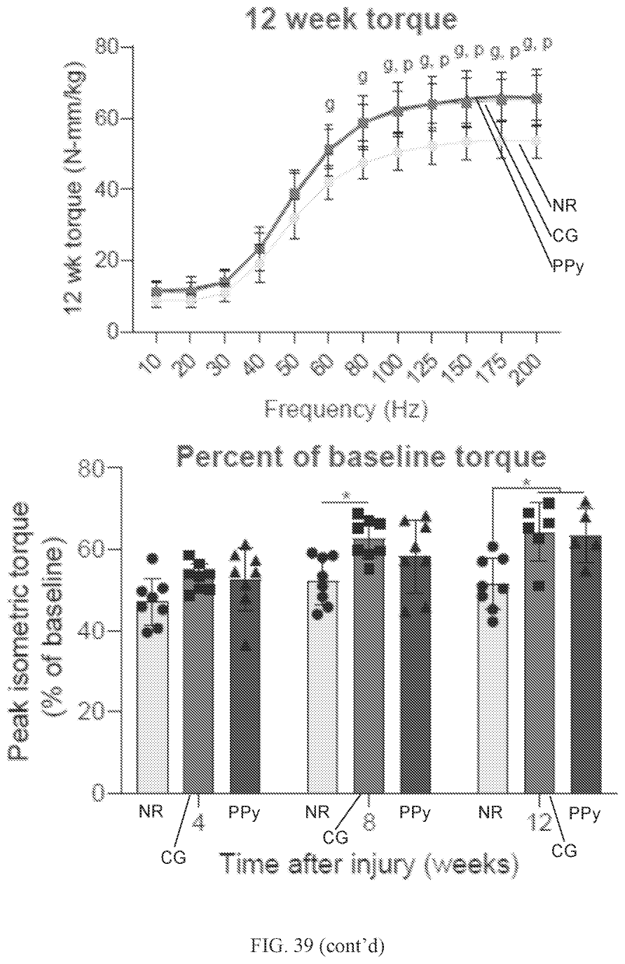

Referring to FIG. 39, functional recovery of TA muscles was measured by isometric muscle contraction (see FIG. 38). Baseline isometric torque measurements were not significantly different between groups but displayed a marked reduction in force production 4 weeks post injury. At 8 weeks non-conductive CG scaffolds showed an increase in torque production at higher stimulation frequencies compared to no repair muscles. At 12 weeks post VML both PPy-doped and non-conductive CG scaffolds showed an increase in isometric torque compared to no repair muscles. Statistically significant differences from no repair are denoted by g (CG) and p (PPy). When isometric torque was normalized to baseline values PPy-doped and non-conductive CG scaffolds supported improved functional recovery compared to non-treated muscles. Data presented as Mean+/−SD.

This EXAMPLE shows that scaffolds with graded biophysical properties, reminiscent of the MTJ, can be fabricated via a multi-compartment layering technique and directional lyophilization. It further shows that multi-compartment scaffolds can contain homogenous pore alignment and architecture. It also shows that both conductive and non-conductive CG scaffolds, or “single compartments,” support improved functional muscle recovery in an in vivo model of VML.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Albuquerque et al. Mammalian Nicotinic Acetylcholine Receptors: From Structure to Function. Physiol Rev. American Physiological Society; 2009 Jan. 1; 89(1):73-120.
Alegret et al. 3D Scaffolds Based on Conductive Polymers for Biomedical Applications. Biomacromolecules, 2019, 20, 73-89.
Ateh et al. Polypyrrole-based conducting polymers and interactions with biological tissues. J. R. Soc. Interface, 2006, 3, 741-752.
Baker et al. Cell and Growth Factor-Loaded Keratin Hydrogels for Treatment of Volumetric Muscle Loss in a Mouse Model. Tissue Eng. Part A, 2017, 23, 572-584.
Balint et al. Conductive polymers: towards a smart biomaterial for tissue engineering. Acta Biomater., 2014, 10, 2341-2353.
Basurto et al. Aligned and Conductive 3D Collagen Scaffolds for Skeletal Muscle Tissue Engineering. bioRxiv. 2020 Jan. 1; 2020.04.18.048017.
Beltran et al. Fate of Combat Nerve Injury. J Orthop Trauma. 2012; 26(11):e198-e203.
Bentzinger et al. Cellular dynamics in the muscle satellite cell niche. EMBO Rep., 2013, 14, 1062-1072.
Browe & Freeman. Optimizing C2C12 myoblast differentiation using polycaprolactone-polypyrrole copolymer scaffolds. J. Biomed. Mater. Res. A, 2019, 107, 220-231.
Brown et al. Macrophage phenotype and remodeling outcomes in response to biologic scaffolds with and without a cellular component. Biomaterials. 2009; 30(8):1482-91. PMID: 19121538
Brown et al. Rethinking regenerative medicine: a macrophage-centered approach. Front Immunol. Frontiers Media S.A.; 2014 Nov. 4; 5:510-510.
Burnstine-Townley et al. Conductive Scaffolds for Cardiac and Neuronal Tissue Engineering: Governing Factors and Mechanisms. Adv. Funct. Mater., 2020, 30, 1901369.
Bursac et al. Synergizing Engineering and Biology to Treat and Model Skeletal Muscle Injury and Disease. Annu. Rev. Biomed. Eng., 2015, 17, 217-242.
Caliari & Harley. The effect of anisotropic collagen-GAG scaffolds and growth factor supplementation on tendon cell recruitment, alignment, and metabolic activity. Biomaterials. 2011 Aug. 1; 32(23):5330-5340. PMCID: PMC3947515
Caliari et al. The development of collagen-GAG scaffold-membrane composites for tendon tissue engineering. Biomaterials, 2011, 32, 8990-8998.
Caliari et al. The influence of collagen-glycosaminoglycan scaffold relative density and microstructural anisotropy on tenocyte bioactivity and transcriptomic stability. Spec Issue Tissue Eng. 2012 Jul. 1; 11:27-40.
Caliari et al. The influence of collagen-glycosaminoglycan scaffold relative density and microstructural anisotropy on tenocyte bioactivity and transcriptomic stability. J. Mech. Behav. Biomed. Mater., 2012, 11, 27-40.
Caliari et al. Collagen Scaffold Arrays for Combinatorial Screening of Biophysical and Biochemical Regulators of Cell Behavior. Adv Healthc Mater. 2015 Jan. 1; 4(1):58-64.
Caliari et al. Collagen Scaffolds Incorporating Coincident Gradations of Instructive Structural and Biochemical Cues for Osteotendinous Junction Engineering. Adv Healthc Mater. 2015b April 1; 4(6):831-837. PMCID: PMC4409544
Carlson. The biology of long-term denervated skeletal muscle. Eur J Transl Myol. 2014; 24(1):5-11.
Carlson & Faulkner. The regeneration of skeletal muscle fibers following injury: a review. Med. Sci. Sports Exerc., 1983, 15, 187-198.
Charvet et al. The development of the myotendinous junction. A review. Muscles Ligaments Tendons J. 2012; 2(2):53-63. PMID: 23738275
Chen et al. Electrically conductive nanofibers with highly oriented structures and their potential application in skeletal muscle tissue engineering. Acta Biomater., 2013, 9, 5562-5572.
Chen et al. Electrical stimulation as a novel tool for regulating cell behavior in tissue engineering. Biomater Res. Biomaterials Research; 2019; 23(1):1-12.
Clarke. Swing time changes contribute to stride time adjustment in the walking rat. Physiol Behav. 1991 Dec. 1; 50(6):1261-1262.
Corona et al. Further Development of a Tissue Engineered Muscle Repair Construct In Vitro for Enhanced Functional Recovery Following Implantation In Vivo in a Murine Model of Volumetric Muscle Loss Injury. Tissue Eng Part A. 2012 Mar. 22; 18(11-12):1213-1228. PMCID: PMC3360508
Corona et al. Implantation of In Vitro Tissue Engineered Muscle Repair Constructs and Bladder Acellular Matrices Partially Restore In Vivo Skeletal Muscle Function in a Rat Model of Volumetric Muscle Loss Injury. Tissue Eng Part A. 2013 Sep. 25; 20(3-4):705-715. PMCID: PMC4518882
Corona et al. Volumetric muscle loss leads to permanent disability following extremity trauma. J Rehabil Res Dev. 2015 Jun. 18; 52(7):785-792.
Corona et al. Pathophysiology of Volumetric Muscle Loss Injury. Cells Tissues Organs, 2016, 202, 180-188.
de Ruiter et al. Designing ideal conduits for peripheral nerve repair. Neurosurg. Focus, 2009, 26, E5.
Decaris & Leach. Design of Experiments Approach to Engineer Cell-Secreted Matrices for Directing Osteogenic Differentiation. Ann Biomed Eng. 2011 Apr. 1; 39(4):1174-1185.
Dienes et al. Analysis and Modeling of Rat Gait Biomechanical Deficits in Response to Volumetric Muscle Loss Injury. Front Bioeng Biotechnol. 2019; 7:146.
Divakar et al. Anisotropic freeze-cast collagen scaffolds for tissue regeneration: How processing conditions affect structure and properties in the dry and fully hydrated states. J. Mech. Behav. Biomed. Mater., 2019, 90, 350-364.
Docheva et al. Tenomodulin Is Necessary for Tenocyte Proliferation and Tendon Maturation. Mol Cell Biol. 2005 Jan. 15; 25(2):699.
Donnelly et al. A Novel bioreactor for stimulating skeletal muscle in vitro. Tissue Eng—Part C Methods. 2010; 16(4):711-718.
Elias et al. In Vivo Evaluation of the Genotoxic Effects of Poly (Butylene adipate-co-terephthalate)/Polypyrrole with Nanohydroxyapatite Scaffolds for Bone Regeneration. Materials. 2019; 12(8):1330.
Engler et al. Matrix Elasticity Directs Stem Cell Lineage Specification. Cell. 2006 Aug. 25; 126(4):677-689. PMID: 16923388
Fennelly C, Soker S. Bioelectric Properties of Myogenic Progenitor Cells. Bioelectricity. Mary Ann Liebert, Inc., publishers; 2019 Mar. 1; 1(1):35-45.
Fleischer et al. Coiled fiber scaffolds embedded with gold nanoparticles improve the performance of engineered cardiac tissues. Nanoscale, 2014, 6, 9410-9414.
Fu et al. Sulfur-polypyrrole composite cathodes for lithium-sulfur batteries. Electrochem. Soc., 2012, 159, A1420-A1424.
Gabriel (Internet), Compilation of the dielectric properties of body tissues at RF and microwave frequencies, https://itis.swiss/virtual-population/tissue-properties/database/dielectric-properties/.
Gattazzo et al. Extracellular matrix: a dynamic microenvironment for stem cell niche. Biochem Biophys Acta. 2014, 8: 2506-19.
Getgood et al. Evaluation of early-stage osteochondral defect repair using a biphasic scaffold based on a collagen-glycosaminoglycan biopolymer in a caprine model. The Knee. 2012 Aug. 1; 19(4):422-430.
Gibson & Ashby. Cellular Solids: Structure and Properties, Cambridge University Press, Cambridge, 2nd edn., 1997.
Gilbert et al. Substrate Elasticity Regulates Skeletal Muscle Stem Cell Self-Renewal in Culture. Science. 2010 Aug. 27; 329(5995):1078-1081. PMID: 20647425
Gilbert-Honick & Grayson. Vascularized and Innervated Skeletal Muscle Tissue Engineering. Adv Healthc Mater. 2019; 1900626:1-27.
Gillies & Lieber. Structure and Function of the Skeletal Muscle Extracellular Matrix. Muscle Nerve. 2011 Sep. 1; 44(3):318-331. PMCID: PMC3177172
Gilmore et al. Skeletal muscle cell proliferation and differentiation on polypyrrole substrates doped with extracellular matrix components. Biomaterials, 2009, 30, 5292-5304.
Godard et al. Designing ideal conduits for peripheral nerve repair. Neurosurg Focus FOC. American Association of Neurological Surgeons; 2009; 26(2):E5.
Goldberg. Work-induced growth of skeletal muscle in normal and hypophysectomized rats. Am J Physiol. 1967; 213(5):1193-1198. PMID: 6054866
Goldberg. Death and Injury Rates of U.S. Military Personnel in Iraq. Mil Med. 2010 Apr. 1; 175(4):220-226.
Goldspink. Changes in muscle mass and phenotype and the expression of autocrine and systemic growth factors by muscle in response to stretch and overload. J Anat. 1999; 194(3):323-334.
Grogan & Hsu. Volumetric muscle loss. JAAOS—J. Am. Acad. Orthop. Surg., 2011, 19, S35-S37.
Gross & Hoffmann. Therapeutic Strategies for Tendon Healing Based on Novel Biomaterials, Factors and Cells. Pathobiology. 2013; 80(4):203-210. PMID: 23652284
Guo & Ma. Conducting Polymers for Tissue Engineering. Biomacromolecules, 2018, 19, 1764-1782.
Hall. Textbook of Medical Physiology, 13th Edition, Elsevier, 2016.
Hanif et al. Development of a vapor phase polymerization method using a wet-on-wet process to coat polypyrrole on never-dried nanocellulose crystals for fabrication of compression strain sensor. Chem Eng J. 2020 Feb. 1; 381:122700.
Harley & Gibson. In vivo and in vitro applications of collagen-GAG scaffolds. Chem. Eng. J., 2008, 137, 102-121.
Harley et al. Optimal Degradation Rate for Collagen Chambers Used for Regeneration of Peripheral Nerves over Long Gaps. Cells Tissues Organs. 2004; 176(1-3):153-165.
Harley et al. Design of a multiphase osteochondral scaffold III: Fabrication of layered scaffolds with continuous interfaces. J Biomed Mater Res A. 2009 Mar. 19; 92A(3):1078-1093.
Harley & Gibson. In vivo and in vitro applications of collagen-GAG scaffolds. Chem Eng J. 2008; 137(1):102-121.
Hernández et al. Electrical stimulation promotes cardiac differentiation of human induced pluripotent stem cells. Stem Cells Int. 2016: Article ID 1718041.
Hsiao et al. Electrical coupling of isolated cardiomyocyte clusters grown on aligned conductive nanofibrous meshes for their synchronized beating. Biomaterials, 2013, 34, 1063-1072.
Hui et al. Spatiotemporal Control of Viscoelasticity in Phototunable Hyaluronic Acid Hydrogels. Biomacromolecules. 2019 Nov. 11; 20(11):4126-4134.
Juhas et al. Biomimetic engineered muscle with capacity for vascular integration and functional maturation in vivo. Proc. Natl. Acad. Sci. U.S.A., 2014, 111, 5508-5513.
Juhas et al. Incorporation of macrophages into engineered skeletal muscle enables enhanced muscle regeneration. Nat Biomed Eng. 2018; 2(12):942-954.
Kasper et al. Mimicking exercise in three-dimensional bioengineered skeletal muscle to investigate cellular and molecular mechanisms of physiological adaptation. J Cell Physiol. 2018; 233(3):1985-1998.
Khodabukus & Baar. Defined electrical stimulation emphasizing excitability for the development and testing of engineered skeletal muscle. Tissue Eng—Part C Methods. 2012; 18(5):349-357.
Kim et al. Neural cell integration into 3D bioprinted skeletal muscle constructs accelerates restoration of muscle function. Nat Commun. 2020 Feb. 24; 11(1):1025.
Ko et al. Promotion of Myogenic Maturation by Timely Application of Electric Field Along the Topographical Alignment. Tissue Eng—Part A. 2018; 24(9-10):752-760.
Ku et al. Synergic effects of nanofiber alignment and electroactivity on myoblast differentiation. Biomaterials, 2012, 33, 6098-6104.
Ladd et al. Co-electrospun dual scaffolding system with potential for muscle-tendon junction tissue engineering. Biomaterials. 2011 Feb. 1; 32(6):1549-1559. MED: 21093046
Larkin et al. Structure and Functional Evaluation of Tendon-Skeletal Muscle Constructs Engineered in Vitro. Tissue Eng. 2006 November; 12(11):3149-3158. PMCID: PMC2798802
Lee & Schmidt. Amine-functionalized polypyrrole: Inherently cell adhesive conducting polymer. J Biomed Mater Res A. 2015 Jun. 1; 103(6):2126-2132. PMCID: PMC4390405
Lee et al. Carboxylic acid-functionalized conductive polypyrrole as a bioactive platform for cell adhesion. Biomacromolecules, 2006, 7, 1692-1695.
Lesman et al. Engineering vessel-like networks within multicellular fibrin-based constructs. Biomaterials, 2011, 32, 7856-7869.
Levin et al. The importance of factorial design in tissue engineering and biomaterials science: Optimisation of cell seeding efficiency on dermal scaffolds as a case study. J Tissue Eng. 2018 Jan. 1; 9:2041731418781696.
Liao et al. Effect of Electromechanical Stimulation on the Maturation of Myotubes on Aligned Electrospun Fibers. Cell Mol Bioeng. 2008; 1(2-3):133-145.
Liu et al. ole of an inward rectifier K+ current and of hyperpolarization in human myoblast fusion. J. Physiol., 1998, 510, 467-476.
Machingal et al. A Tissue-Engineered Muscle Repair Construct for Functional Restoration of an Irrecoverable Muscle Injury in a Murine Model. Tissue Eng Part A. 2011 May 6; 17(17-18):2291-2303. PMCID: PMC3161107
Maleiner et al. The importance of biophysical and biochemical stimuli in dynamic skeletal muscle models. Front Physiol. 2018; 9(AUG):1-24.
Martin et al. Neuromuscular junction formation in tissue-engineered skeletal muscle augments contractile function and improves cytoskeletal organization. Tissue Eng—Part A. 2015; 21(19-20):2595-2604.
Martin et al. The generation of biomolecular patterns in highly porous collagen-GAG scaffolds using direct photolithography. Biomaterials. 2011 Jun. 1; 32(16):3949-3957. PMCID: PMC3947768
McClure et al. Decellularized Muscle Supports New Muscle Fibers and Improves Function Following Volumetric Injury. Tissue Eng. Part A, 2018, 24, 1228-1241.
McLaughlin & Levin. Bioelectric signaling in regeneration: Mechanisms of ionic controls of growth and form. Dev Biol. Elsevier Inc.; 2018; 433(2):177-189.
Menezes & Luskin. Expression of neuron-specific tubulin defines a novel population in the proliferative layers of the developing telencephalon. J Neurosci. 1994 Sep. 1; 14(9):5399.
Menorca et al. Peripheral Nerve Trauma: Mechanisms of Injury and Recovery. Hand Clin. 2013; 29(3):317-330. PMID: 23895713
Mihic et al. A conductive polymer hydrogel supports cell electrical signaling and improves cardiac function after implantation into myocardial infarct. Circulation. 2015; 132(8):772-784.
Mintz et al. Long-Term Evaluation of Functional Outcomes Following Rat Volumetric Muscle Loss Injury and Repair. Tissue Eng Part A. Mary Ann Liebert, Inc., publishers; 2019 Oct. 3; 26(3-4):140-156.
Mintz et al. Applications of In Vivo Functional Testing of the Rat Tibialis Anterior for Evaluating Tissue Engineered Skeletal Muscle Repair. JoVE J Vis Exp. 2016 Oct. 7; (116):e54487-e54487. PMCID: PMC5092182
Moon et al. Cyclic Mechanical Preconditioning Improves Engineered Muscle Contraction. Tissue Eng Part A. Mary Ann Liebert, Inc., publishers; 2008 Apr. 1; 14(4):473-482.
Morgan & Black. Mimicking isovolumic contraction with combined electromechanical stimulation improves the development of engineered cardiac constructs. Tissue Eng—Part A. 2014; 20(11-12):1654-1667.
Murphy et al. The effect of mean pore size on cell attachment, proliferation and migration in collagen-glycosaminoglycan scaffolds for bone tissue engineering. Biomaterials, 2010, 31, 461-466.
Musumeci et al. Somitogenesis: From somite to skeletal muscle. Acta Histochem. Elsevier GmbH.; 2015; 117(4-5):313-328. PMID: 25850375
Nair et al. Self-assembly of collagen bundles and enhanced piezoelectricity induced by chemical crosslinking. Nanoscale, 2019, 11, 15120-15130.
Nakayama et al. Treatment of volumetric muscle loss in mice using nanofibrillar scaffolds enhances vascular organization and integration. Commun. Biol., 2019, 2:170.
Navaei et al. The influence of electrically conductive and non-conductive nanocomposite scaffolds on the maturation and excitability of engineered cardiac tissues. Biomater. Sci., 2019, 7, 585-595.
Neal et al. Mechanical Characterization and Shape Optimization of Fascicle-Like 3D Skeletal Muscle Tissues Contracted with Electrical and Optical Stimuli. Tissue Eng. Part A, 2015, 21, 1848-1858.
Nishimura. Role of extracellular matrix in development of skeletal muscle and postmortem aging of meat. Meat Sci. Elsevier Ltd; 2015; 109:48-55.
Niu et al. Enhancing neural differentiation of induced pluripotent stem cells by conductive graphene/silk fibroin films. Biomed. Mater. Res. A, 2018, 106, 2973-2983.
O'Brien et al. The effect of pore size on cell adhesion in collagen-GAG scaffolds. Biomaterials. 2005 Feb. 1; 26(4):433-441.
Olde Damink et al. Cross-linking of dermal sheep collagen using a water-soluble carbodiimide. Biomaterials. 1996 Jan. 1; 17(8):765-773. PMID: 8730960
Owens et al. Characterization of Extremity Wounds in Operation Iraqi Freedom and Operation Enduring Freedom. J Orthop Trauma 2007; 21(4):254-257.
Owens et al. Combat wounds in operation iraqi freedom and operation enduring freedom. J Trauma—Inj Infect Crit Care. 2008; 64(2):295-299. PMID: 18301189
Passipieri et al. Keratin Hydrogel Enhances In Vivo Skeletal Muscle Function in a Rat Model of Volumetric Muscle Loss. Tissue Eng Part A. 2017 Feb. 7; 23(11-12):556-571. PMID: 28169594
Passipieri et al. In Silico and In Vivo Studies Detect Functional Repair Mechanisms in a Volumetric Muscle Loss Injury. Tissue Eng Part A. 2019 Mar. 18; 25(17-18):1272-1288.
Patel et al. Biomed. Mater., 2019, 14, 035010.
Pawelec et al. Ice-templated structures for biomedical tissue repair: From physics to final scaffolds. Appl Phys Rev. American Institute of Physics; 2014 Apr. 11; 1(2):021301.
Pek et al. Degradation of a collagen-chondroitin-6-sulfate matrix by collagenase and by chondroitinase. Biomaterials. 2004; 25(3):473-482.
Pelto et al. Novel polypyrrole-coated polylactide scaffolds enhance adipose stem cell proliferation and early osteogenic differentiation. Tissue Eng. Part A, 2013, 19, 882-892.
Perry et al. Genetically engineered human muscle transplant enhances murine host neovascularization and myogenesis. Commun. Biol., 2018, 1, 161.
Pollot & Corona. Volumetric Muscle Loss. Methods Mol. Biol., 2016, 1460, 19-31.
Qazi et al. Tissue engineering of electrically responsive tissues using polyaniline based polymers: a review. Biomaterials, 2014, 35, 9068-9086.
Quarta et al. Bioengineered constructs combined with exercise enhance stem cell-mediated treatment of volumetric muscle loss. Nat. Commun., 2017, 8, 1-17.
Raimondo & Mooney. Functional muscle recovery with nanoparticle-directed M2 macrophage polarization in mice. Proc Natl Acad Sci. 2018; 115(42):10648-10653.
Ramanaviciene et al. Biocompatibility of polypyrrole particles: an in-vivo study in mice. J. Pharm. Pharmacol., 2007, 59, 311-315.
Rao et al. Engineering human pluripotent stem cells into a functional skeletal muscle tissue. Nat Commun. Springer US; 2018; 9(1):126.
Rehmann et al. Tuning microenvironment modulus and biochemical composition promotes human mesenchymal stem cell tenogenic differentiation. J Biomed Mater Res A. 2016 May; 104(5):1162-1174.
Ryan et al. Electroconductive Biohybrid Collagen/Pristine Graphene Composite Biomaterials with Enhanced Biological Activity. Adv. Mater., 2018, 30, e1706442.
Saclier et al. Differentially activated macrophages orchestrate myogenic precursor cell fate during human skeletal muscle regeneration. Stem Cells. 2013; 31(2):384-396.
Sarrafian et al. Extracellular matrix scaffolds for treatment of large volume muscle injuries: A review. Vet Surg. 2018; 47(4):524-535.
Schmidt et al. Stimulation of neurite outgrowth using an electrically conducting polymer. Proc Natl Acad Sci. 1997 Aug. 19; 94(17):8948-8953. PMID: 9256415
Schweitzer et al. Analysis of the tendon cell fate using Scleraxis, a specific marker for tendons and ligaments. Development. 2001 Oct. 1; 128(19):3855.
Severt et al. Versatile Method for Producing 2D and 3D Conductive Biomaterial Composites Using Sequential Chemical and Electrochemical Polymerization. ACS Appl. Mater. Interfaces, 2015, 7, 25281-25288.
Shin et al. Carbon-nanotube-embedded hydrogel sheets for engineering cardiac constructs and bioactuators. ACS Nano, 2013, 7, 2369-2380.
Sirivisoot et al. Protocol and cell responses in three-dimensional conductive collagen gel scaffolds with conductive polymer nanofibres for tissue regeneration. Interface Focus, 2013, 4, 20130050-20130050.
Skalec et al. Anatomy and histology of the rabbit common calcanean tendon. J Vet Med Ser C Anat Histol Embryol. 2019; 48(5):466-475.
Subramanian et al. Axially aligned electrically conducting biodegradable nanofibers for neural regeneration. Mater. Sci. Mater. Med., 2012, 23, 1797-1809.
Sun et al. Carbon nanotube-incorporated collagen hydrogels improve cell alignment and the performance of cardiac constructs. Int. J. Nanomedicine, 2017, 12, 3109-3120.
Sun et al. Polypyrrole-coated poly(l-lactic acid-co-ε-caprolactone)/silk fibroin nanofibrous nerve guidance conduit induced nerve regeneration in rat. Mater Sci Eng C. Elsevier; 2019; 94(August 2017):190-199.
Tadros et al. Muscle-Tendon-Enthesis Unit. Semin Musculoskelet Radiol. 2018; 22(3):263-274.
Tapscott. The circuitry of a master switch: Myod and the regulation of skeletal muscle gene transcription. Development. 2005 Jun. 15; 132(12):2685-2695. PMID: 15930108
Tayebi et al. 3D conductive nanocomposite scaffold for bone tissue engineering. Int. J. Nanomedicine, 2013, 9, 167
Tidball et al. Site and mechanical conditions for failure of skeletal muscle in experimental strain injuries. J Appl Physiol. 1993 Mar. 1; 74(3):1280-1286. PMID: 8482668
Trotter. Structure-function considerations of muscle-tendon junctions. Comp Biochem Physiol A Mol Integr Physiol. 2002 Dec. 1; 133(4):1127-1133. PMID: 12485696
Turner et al. Localization of paxillin, a focal adhesion protein, to smooth muscle dense plaques, and the myotendinous and neuromuscular junctions of skeletal muscle. Exp Cell Res. 1991 Feb. 1; 192(2):651-655.
Turner & Badylak. Biologic scaffolds for musculotendinous tissue repair. Eur Cell Mater. 2012; 25:130-143.
U. S. B. and J. Initiative, The Burden of Musculoskeletal Diseases in the United States, https://www.boneandjointburden.org/search/node/Muscle.
Vandenburgh et al. Drug-screening platform based on the contractility of tissue-engineered muscle. Muscle Nerve, 2008, 37, 438-447.
Wan et al. 3D Conducting Polymer Platforms for Electrical Control of Protein Conformation and Cellular Functions. J. Mater. Chem. B, 2015, 3, 5040-5048.
Wang et al. Polypyrrole nanoparticles and dye absorption properties. Synth. Met., 2005, 151, 136-140.
Wang et al. Neural stem cell proliferation and differentiation in the conductive PEDOT-HA/Cs/Gel scaffold for neural tissue engineering. Biomater. Sci., 2017, 5, 2024-2034.
Weisgerber et al. Evaluation of multi-scale mineralized collagen—polycaprolactone composites for bone tissue engineering. J Mech Behav Biomed Mater. Elsevier; 2016; 61:318-327.
Weiss et al. Electrical Stimulation and Wound Healing. Arch Dermatol. 1990 Feb. 1; 126(2):222-225.
Williams & Doherty. A preliminary assessment of poly (pyrrole) in nerve guide studies. J. Mater. Sci. Mater. Med., 1994, 5, 429-433.
Yang & Temenoff. Engineering Orthopedic Tissue Interfaces. Tissue Eng Part B Rev. 2009 Feb. 10; 15(2):127-141. PMCID: PMC2817661
Yang et al. Polypyrrole-incorporated conductive hyaluronic acid hydrogels. Biomater Res. Biomaterials Research; 2016a; 20(1):1-7.
Yang et al. Polypyrrole/Alginate Hybrid Hydrogels: Electrically Conductive and Soft Biomaterials for Human Mesenchymal Stem Cell Culture and Potential Neural Tissue Engineering Applications. Macromol. Biosci., 2016b, 16, 1653-1661.
Yannas & Burke. Design of an artificial skin. I. Basic design principles. J. Biomed. Mater. Res., 1980, 14, 65-81.
Yannas & Tzeranis. So PTC. Regeneration of injured skin and peripheral nerves requires control of wound contraction, not scar formation. Wound Repair Regen. John Wiley & Sons, Ltd; 2017 Apr. 1; 25(2):177-191.
Yannas et al. Synthesis and characterization of a model extracellular matrix that induces partial regeneration of adult mammalian skin. Proc Natl Acad Sci USA. 1989; 86(3):933-937. PMID: 2915988
Yazdimamaghani et al. Biomineralization and biocompatibility studies of bone conductive scaffolds containing poly(3,4-ethylenedioxythiophene):poly(4-styrene sulfonate) (PEDOT:PSS). J. Mater. Sci. Mater. Med., 2015, 26, 1-11.
Zhang et al. In vitro biocompatibility study of electrically conductive polypyrrole-coated polyester fabrics. J. Biomed. Res., 2001, 57, 63-71.
Zhang et al. Electrical stimulation of adipose-derived mesenchymal stem cells in conductive scaffolds and the roles of voltage-gated ion channels. Acta Biomater., 2016, 32, 46-56.

It will be understood that various details of the presently disclosed subject matter may be changed without departing from the scope of the presently disclosed subject matter. Furthermore, the foregoing description is for the purpose of illustration only, and not for the purpose of limitation.

1. A multicompartment conductive collagen scaffold composite, comprising a scaffold comprising collagen and an electrically conductive material, optionally wherein the electrically conductive material comprises electrically conductive particles, and further comprising longitudinally aligned pores.

2. The multicompartment conductive collagen scaffold composite of claim 1, further comprising a first compartment comprising a first collagen scaffold and an electrically conductive material, optionally wherein the electrically conductive material comprises electrically conductive particles, and a second compartment comprising a second collagen scaffold, and wherein the second compartment is disposed on the first compartment and the pores are longitudinally aligned between the compartments.

3. The multicompartment conductive collagen scaffold composite of claim 1, wherein the scaffold comprising collagen, the first collagen scaffold and/or the second collagen scaffold comprises collagen-glycosaminoglycan (CG).

4. The multicompartment conductive collagen scaffold composite of claim 2, wherein a collagen concentration of the first collagen scaffold varies as compared to a collagen concentration of the second collagen scaffold.

5. The multicompartment conductive collagen scaffold composite of claim 2, wherein the collagen concentration for the first collagen scaffold ranges from about 0.5 weight percent (wt %) to about 1.5 wt % and the collagen concentration for the second collagen ranges from about 1.5 wt % to about 5 wt %.

6. The multicompartment conductive collagen scaffold composite of claim 1, wherein the electrically conductive particles are microparticles.

7. The multicompartment conductive collagen scaffold composite of claim 1, wherein the electrically conductive material is present in an amount ranging from about from about 0.1 wt % to about 3 wt % and/or comprises electrically conductive polypyrrole, poly(3,4-ethylenedioxythiophene) (PEDOT), polyaniline, or combinations thereof.

8. A method for making a multicompartment conductive collagen scaffold composite, the method comprising:
providing a scaffold comprising collagen;
contacting the scaffold comprising collagen with an electrically conductive material, optionally wherein the electrically conductive material comprises electrically conductive particles, to form a composite comprising the scaffold comprising collagen and the electrically conductive particles; and
freeze-drying the composite to form a multicompartment conductive collagen scaffold composite comprising longitudinally aligned pores.

9. The method of claim 8, further comprising:
providing a first compartment comprising a first collagen scaffold and an electrically conductive material, optionally wherein the electrically conductive material comprises electrically conductive particles, and a second compartment comprising a second collagen scaffold;
contacting the first compartment and the second compartment to form a composite comprising the first compartment and the second compartment; and
freeze-drying the composite to form a multicompartment conductive collagen scaffold composite comprising longitudinally aligned pores.

10. The method of claim 8, wherein the scaffold comprising collagen, the first collagen scaffold and/or the second collagen scaffold comprises collagen-glycosaminoglycan (CG).

11. The method of claim 9, wherein a collagen concentration of the first collagen scaffold varies as compared to a collagen concentration of the second scaffold.

12. The method of claim 9, wherein the collagen concentration for the first collagen scaffold ranges from about 0.5 wt % to about 1.5 wt % and the collagen concentration for the second collagen scaffold ranges from about 1.5 wt % to about 5 wt %.

13. The method of claim 8, wherein the electrically conductive particles are microparticles.

14. The method of claim 8, wherein the electrically conductive material is present in an amount ranging from about from about 0.1 wt % to about 3 wt % and/or comprises electrically conductive polypyrrole, poly(3,4-ethylenedioxythiophene) (PEDOT), polyaniline, or combinations thereof.

15. The method of claim 8, wherein contacting the scaffold comprising collagen with the electrically conductive material, optionally wherein the electrically conductive material comprises electrically conductive particles, comprises layering or coating the scaffold comprising collagen with the electrically conductive particles.

16. The method of claim 9, wherein contacting the first compartment and the second compartment to form a composite comprising the first compartment and the second compartment comprises layering two different suspensions on top of one another, wherein a first suspension of the two different suspensions comprises a first collagen scaffold and an electrically conductive material, optionally wherein the electrically conductive material comprises electrically conductive particles, and a second suspension of the two different suspension comprises a second collagen scaffold.

17. The method of claim 16, comprising allowing the suspensions to interdiffuse prior to freeze-drying, optionally wherein an interdiffusion time ranges from about 15 minutes to 60 minutes.

18. The method of claim 16, wherein the first and second suspensions are prepared by mixing type I collagen, chondroitin sulfate, and acetic acid.

19. The method of claim 8, wherein the freeze drying comprises directional lyophilization.

20. A method of treating a skeletal muscle injury in a subject in need thereof, the method comprising providing a multicompartment conductive collagen scaffold composite, comprising a scaffold comprising collagen and an electrically conductive material, optionally wherein the electrically conductive material comprises electrically conductive particles, and further comprising longitudinally aligned pores; and implanting the multicompartment conductive collagen scaffold composite at a site of the skeletal muscle injury in the subject.

21. The method of claim 20, wherein the multicompartment conductive collagen scaffold composite further comprises a first compartment comprising a first collagen scaffold and an electrically conductive material, optionally wherein the electrically conductive material comprises electrically conductive particles, and a second compartment comprising a second collagen scaffold, and wherein the second compartment is disposed on the first compartment and the pores are longitudinally aligned between the compartments

22. The method of claim 20, wherein the scaffold comprising collagen, the first collagen scaffold and/or the second collagen scaffold comprises collagen-glycosaminoglycan (CG).

23. The method of claim 21, wherein a collagen concentration of the first collagen scaffold varies as compared to a collagen concentration of the second scaffold.

24. The method of claim 23, wherein the collagen concentration for the first collagen scaffold ranges from about 0.5 wt % to about 1.5 wt % and the collagen concentration for the second collagen scaffold ranges from about 1.5 wt % to about 5 wt %.

25. The method of claim 20, wherein the electrically conductive particles are microparticles.

26. The method of claim 20, wherein the electrically conductive material is present in an amount ranging from about from about 0.1 wt % to about 3 wt % and/or comprise electrically conductive polypyrrole, poly(3,4-ethylenedioxythiophene) (PEDOT), polyaniline, or combinations thereof.

27. The method of claim 20, wherein the skeletal muscle injury comprises an injury at a muscle-tendon junction (MTJ).

28. The multicompartment conductive collagen scaffold composite of claim 1, wherein pore size ranges from about 50 μm to about 250 μm in diameter 29.

29. The multicompartment conductive collagen scaffold composite of claim 1, wherein the pores are elongated.

30. The multicompartment conductive collagen scaffold composite of claim 1, wherein cells are seeded to the composite, optionally wherein the cells comprise muscle-derived cells (including myoblasts and satellite cells), fibroblasts, neural cells (including neural stem cells, motor neurons), and combinations thereof.

31. The method of claim 8, wherein pore size ranges from about 50 μm to about 250 μm in diameter.

32. The method of claim 8, wherein the pores are elongated.

33. The method of claim 8, wherein cells are seeded to the composite, optionally wherein the cells comprise muscle-derived cells (including myoblasts and satellite cells), fibroblasts, neural cells (including neural stem cells, motor neurons), and combinations thereof.
